(19) United States
(12) Patent Application Publication
(10) Pub. No.: US 2020/0237700 A1
(43) Pub. Date: Jul. 30, 2020

(54) METHODS OF REDUCING OR PREVENTING OXIDATION OF SMALL DENSE LDL OR MEMBRANE POLYUNSATURATED FATTY ACIDS

(71) Applicant: Amarin Pharmaceuticals Ireland Limited, Dublin (IE)

(72) Inventor: Richard Preston Mason, Bridgewater, NJ (US)

(21) Appl. No.: 16/846,870

(22) Filed: Apr. 13, 2020

Related U.S. Application Data
(62) Division of application No. 15/258,565, filed on Sep. 7, 2016.
(60) Provisional application No. 62/216,013, filed on Sep. 9, 2015.

(51) Int. Cl. A61K 31/202; A61K 31/40

(52) U.S. Cl. CPC A61K 31/202; A61K 31/40

(57) ABSTRACT

In various embodiments, the present invention provides methods of treating and/or preventing cardiovascular-related disease and, in particular, a method of reducing or preventing sdLDL oxidation in a subject, the method comprising administering to the subject a pharmaceutical composition comprising eicosapentaenoic acid or a derivative thereof.

### PRIORITY CLAIM

This application is a divisional application of U.S. patent application Ser. No. 15/258,565 filed Sep. 7, 2016, which claims the benefit of U.S. Provisional Patent Application No. 62/216,013, entitled “Methods of Reducing or Preventing Oxidation of Small Dense LDL or Membrane Polyunsaturated Fatty Acids,” filed Sep. 9, 2015, each of which is incorporated herein in its entirety by this reference thereto.

### BACKGROUND

Cardiovascular disease is one of the leading causes of death in the United States and most European countries. It is estimated that over 70 million people in the United States alone suffer from a cardiovascular disease or disorder including but not limited to high blood pressure, coronary heart disease, dyslipidemia, congestive heart failure and stroke. A need exists for improved treatments for cardiovascular diseases and disorders.

### SUMMARY

In one embodiment, the present invention provides a method of reducing or preventing membrane cholesterol domain formation in a subject in need thereof, the method comprising administering to the subject a pharmaceutical composition comprising eicosapentaenoic acid or a derivative thereof.

In another embodiment, the present invention provides a method of reducing or preventing sdLDL oxidation in a subject in need thereof, the method comprising administering to the subject a pharmaceutical composition comprising eicosapentaenoic acid or a derivative thereof.

In another embodiment, the present invention provides a method of reducing or preventing membrane cholesterol domain formation in a subject in need thereof, the method comprising administering to the subject a pharmaceutical composition comprising eicosapentaenoic acid or a derivative thereof.

In another embodiment, the present invention provides a method of reducing or preventing oxidative modification of membrane polyunsaturated fatty acids in a subject in need thereof, the method comprising administering to the subject a pharmaceutical composition comprising eicosapentaenoic acid or a derivative thereof.

These and other embodiments of the present invention will be disclosed in further detail herein below.

### BRIEF DESCRIPTION OF THE FIGURES

FIG. 1 depicts the effects of ethyl eicosapentaenoate (“EPA”) on glucose-induces membrane lipid peroxidation from 0-96 hours compared to glucose or vehicle control.

FIG. 2 depicts dose-dependent effects of EPA on glucose-induced membrane lipid peroxidation in model membranes.

FIG. 3 shows a comparison of the effects of vitamin E and EPA on glucose-induced membrane lipid peroxidation in model membranes.

FIG. 4 shows representative X-ray diffraction patterns for model membranes prepared in the presence of glucose and treated with vehicle control (top row), vitamin E (middle row), or EPA (bottom row) at 0 hours (left column), 72 hours (middle column) and 96 hours (right column).

FIG. 5 depicts the quantitative assessment of the comparative effects of vitamin E and EPA on glucose- and peroxidation-induced cholesterol domain formation.

FIG. 6 shows a comparison of the combined effects of EPA and atorvastatin o-hydroxy (active) metabolite (“ATM”) to EPA alone and ATM alone on glucose-induced membrane lipid peroxidation.

FIG. 7 depicts a schematic representation of one possible mechanism to explain antioxidant and membrane structural effects of EPA.

FIG. 8 depicts the effects of glucose with or without any one of EA, ETE or EPA on lipid hydroperoxide formation compared to control.

FIG. 9 depicts the effects of glucose with or without any one of EA, ETE or EPA on lipid hydroperoxide formation compared to control after 96 hours.

FIG. 10 depicts the dose-dependent effects of EPA and vitamin E on sdLDL oxidation after 2 hours.

FIG. 11 depicts the dose-dependent antioxidant effects of EPA in human sdLDL compared to non-fractionated LDL.

FIG. 12 shows the dose-dependent effects of EPA on human sdLDL oxidation as measured by colorimetric assay of TBARS formation and expressed as molar equivalents of malondialdehyde (“MDA”). Values are mean+/−S.D. (N=3). **p<0.001 versus vehicle-treated control; ^{†}p<0.001 versus 1.0 μM EPA; ^{§} p<0.001 versus 2.5 μM EPA; ^{¶}p<0.05 versus 5.0 μM EPA (Student-Newman-Keuls multiple comparisons test; overall ANOVA: p<0.0001, F=2960.1).

FIG. 13 shows the dose-dependent effects of atorvastatin o-hydroxy (active) metabolite (“ATM”) on human sdLDL oxidation as measured by colorimetric assay of TBARS formation and expressed as molar equivalents of MDA. Values are mean+/−S.D. (N=3). *p<0.05 and **p<0.001 versus vehicle-treated control; ^{†}p<0.01 versus 0.10 μM ATM; ^{§} p<0.001 versus 0.5 μM ATM; ^{¶}p<0.001 versus 1.0 μM ATM (Student-Newman-Keuls multiple comparisons test; overall ANOVA: p<0.0001, F=745.92).

FIG. 14 shows the separate and combined effects of EPA and ATM on human sdLDL oxidation as measured by colorimetric assay of TBARS formation and expressed as molar equivalents of MDA. Values are mean+/−S.D. (N=3). *p<0.01 and **p<0.001 versus vehicle-treated control; ^{†}p<0.05 and ^{‡}p<0.001 versus EPA; ^{§} p<0.05 and ^{§§} p<0.001 versus 0.5 μM ATM; ^{¶}p<0.001 versus 1.0 μM ATM; ^{u}p<0.001 versus EPA+0.5 μM ATM (Student-Newman-Keuls multiple comparisons test; overall ANOVA: p<0.0001, F=118.22).

FIG. 15 shows the separate and combined effects of EPA (1.0 μM) and ATM on human sdLDL oxidation expressed as percent decrease in MDA formation relative to vehicle-treated control. Data was collected after 1 hour of exposure to oxidative conditions. Values are mean+/−S.D. (N=3). *p<0.01 and **p<0.001 versus vehicle-treated control (Student-Newman-Keuls multiple comparisons test; overall ANOVA: p<0.0001, F=118.22). ^{†}p<0.05 and ^{‡}p<0.001 versus EPA; ^{§} p<0.05 and ^{§§} p<0.001 versus 0.5 μM ATM; ^{¶}p<0.001 versus 1.0 μM ATM; *p<0.001 versus EPA+0.5 μM ATM (Student-Newman-Keuls multiple comparisons test; overall ANOVA: p<0.0001, F=92.011).

FIG. 16 shows the comparative effects of EPA, fenofibrate (“Fenofib”), nicotinic acid (niacin), gemfibrozil (“Gemfib”) and vitamin E (all at 10 μM) on human sdLDL oxidation. Data were collected after 1 hour of exposure to oxidative conditions. Values are mean+/−S.D. (N=3). **p<0.001 versus vehicle-treated control; ^{‡}p<0.001 versus fenofibrate, niacin, or gemfibrozil; ^{§} p<0.001 versus Vit E (Student-Newman-Keuls multiple comparisons test; overall ANOVA: p<0.0001, F=1268.1).

FIG. 17 shows the separate and combined effects of EPA and atorvastatin (“Atorva”) on human sdLDL oxidation. Each agent was tested at 10 μM. Data were collected after 1 hour of exposure to oxidative conditions. Values are mean+/−S.D. (N=3). **p<0.001 versus vehicle-treated control; ^{†}p<0.001 versus atorvastatin alone (Student-Newman-Keuls multiple comparisons test; overall ANOVA: p<0.0001, F=3962.4).

FIG. 18 shows the comparative effects of EPA, fenofibrate, nicotinic acid and gemfibrozil, alone or in combination with atorvastatin, on human sdLDL oxidation. Each agent was tested at 10 μM. Data were collected after 1 hours of exposure to oxidative conditions. Values are mean±S.D. (N=3). **p<0.001 versus vehicle-treated control; ^{‡}p<0.001 versus all other TG-lowering agents, alone or in combination with Atorva (Student-Newman-Keuls multiple comparisons test; overall ANOVA: p<0.0001, F=1365.7).

FIG. 19 shows the comparative effects of EPA and DHA on human sdLDL oxidation. Both agents were tested at 10 μM. Data were collected after 4 hours of exposure to oxidative conditions. Values are mean+/−S.D. (N=3). **p<0.001 versus vehicle-treated control; ^{‡}p<0.001 versus DHA (Student-Newman-Keuls multiple comparisons test; overall ANOVA: p<0.0001, F=211.20).

FIG. 20 shows the comparative effects of EPA and DHA on the rate of lipid oxidation in human sdLDL. Samples were prepared at 100 μg/mL sdLDL and incubated with EPA or DHA (10.0 μM each) for 30 minutes prior to initiating lipid oxidation using 10 μM CuSO₄. Samples were maintained at 37° C. in a shaking water bath for 1 hour. Lipid oxidation was measured by colorimetric assay of TBARS formation and expressed as molar equivalents of MDA. Values shown are the averages calculated from three separate measurements.

FIG. 21 shows the dose-dependent effects of eicosapentaenoic acid (EPA) on human LDL oxidation. Samples were prepared at 100 μg/mL LDL and incubated with test agents (at doses indicated) for 30 minutes prior to initiating lipid oxidation using 10 μM CuSO₄. Samples were maintained at 37° C. in a shaking water bath for 1 hour. Lipid oxidation was measured by colorimetric assay of TBARS formation and expressed as molar equivalents of MDA. Values are mean+/−S.D. (N=3). **p<0.001 versus vehicle-treated control; ^{†}p<0.001 versus 1.0 μM EPA; ^{§} p<0.001 versus 2.5 μM EPA (Student-Newman-Keuls multiple comparisons test; overall ANOVA: p<0.0001, F=298.14).

FIG. 22 shows the dose-dependent effects of atorvastatin o-hydroxy (active) metabolite (ATM) on human LDL oxidation. Samples were prepared at 100 μg/mL LDL and incubated with test agents (at doses indicated) for 30 minutes prior to initiating lipid oxidation using 10 μM CuSO₄. Samples were maintained at 37° C. in a shaking water bath for 1 hour. Lipid oxidation was measured by colorimetric assay of TBARS formation and expressed as molar equivalents of MDA. Values are mean+/−S.D. (N=3). *p<0.05 and **p<0.001 versus vehicle-treated control; ^{†}p<0.05 and ^{‡}p<0.001 versus 0.10 μM ATM; ^{§} p<0.001 versus 0.5 μM ATM; ^{¶}p<0.001 versus 1.0 μM ATM; ^{♦}p<0.05 versus 5.0 μM ATM (Student-Newman-Keuls multiple comparisons test; overall ANOVA: p<0.0001, F=438.91). Values are mean±S.D. (N=3).

FIG. 23 shows the separate and combined effects of EPA and ATM on human LDL oxidation (expressed as molar MDA equivalents). EPA was tested at 1.0 μM. Data were collected after 1 hour of exposure to oxidative conditions. Values are mean+/−S.D. (N=3). **p<0.001 versus vehicle-treated control; ^{†}p<0.05 and ^{‡}p<0.001 versus EPA; ^{§} p<0.001 versus 0.5 μM ATM; ^{¶}p<0.001 versus 1.0 μM ATM; ^{♦}p<0.001 versus EPA+0.5 μM ATM (Student-Newman-Keuls multiple comparisons test; overall ANOVA: p<0.0001, F=142.74).

FIG. 24 shows the separate and combined effects of EPA and ATM on human LDL oxidation (expressed as % decrease in MDA formation relative to vehicle-treated control). EPA was tested at 1.0 μM. Data were collected after 1 hour of exposure to oxidative conditions. Values are mean+/−S.D. (N=3). **p<0.001 versus vehicle-treated control (Student-Newman-Keuls multiple comparisons test; overall ANOVA: p<0.0001, F=142.74). ^{‡}p<0.001 versus EPA; ^{§} p<0.05 and ^{§§} p<0.001 versus 0.5 μM ATM; ^{¶}p<0.001 versus 1.0 μM ATM; ^{♦}p<0.05 versus EPA+0.5 μM ATM (Student-Newman-Keuls multiple comparisons test; overall ANOVA: p<0.0001, F=39.502).

FIG. 25 shows the comparative effects of EPA, fenofibrate, nicotinic acid, gemfibrozil, and vitamin E on human LDL oxidation. All agents were tested at 10 μM. Data were collected after 1 hour of exposure to oxidative conditions. Values are mean+/−S.D. (N=3). **p<0.001 versus vehicle-treated control; ^{†}p<0.01 versus fenofibrate, nicotinic acid, or gemfibrozil; ^{§} p<0.001 versus vitamin E (Student-Newman-Keuls multiple comparisons test; overall ANOVA: p<0.0001, F=132.37).

FIG. 26 shows the separate and combined effects of EPA and atorvastatin on human LDL oxidation. Each agent was tested at 10 μM. Data were collected after 1 hour of exposure to oxidative conditions. Values are mean+/−S.D. (N=3). *p<0.001 versus vehicle-treated control; ^{†}p<0.001 versus atorvastatin alone (Student-Newman-Keuls multiple comparisons test; overall ANOVA: p<0.0001, F=790.78).

FIG. 27 shows the separate and combined effects of EPA and atorvastatin o-hydroxy (active) metabolite (ATM) on the ratio of NO to ONOO⁻ released from HUVECs exposed to oxidized low-density lipoprotein (oxLDL). Values are mean+/−S.D. (N=3-7). *p<0.05 versus vehicle alone (no oxLDL); ^{†}p<0.05 versus oxLDL+vehicle (Student-Newman-Keuls multiple comparisons test; overall ANOVA: p<0.0307, F=4.162).

FIG. 28 shows the separate and combined effects of docosahexaenoic acid (DHA) and ATM on NO release from HUVECs exposed to oxLDL for one hour. Values are mean+/−S.D. (N=4-16). *p<0.01 versus vehicle alone (no oxLDL); ^{†}p<0.05 and ^{‡}p<0.01 versus oxLDL+vehicle (Student-Newman-Keuls multiple comparisons test; overall ANOVA: p=0.0007, F=6.630).

FIG. 29 shows the effects of ATM, alone or in combination with EPA, DHA, fenofibrate (Febofib), nicotinic acid (Niacin), or gemfibrozil (Gemfib) on NO release from HUVECs exposed to oxLDL. Values are mean+/−S.D. (N=4-16). *p<0.05 versus vehicle alone (no oxLDL); ^{†}p<0.01 versus oxLDL+vehicle (Student-Newman-Keuls multiple comparisons test; overall ANOVA: p=0.0080, F=3.309).

FIG. 30 shows the effects of EPA, Fenofib, Niacin and Gemfib, each in combination with ATM, on the ratio of NO to ONOO⁻ release from HUVECs exposed to oxLDL. Values are mean+/−S.D. (N=3-7). *p<0.05 versus vehicle alone (no oxLDL); ^{†}p<0.05 versus oxLDL+vehicle (Student-Newman-Keuls multiple comparisons test; overall ANOVA: p=0.0188, F=4.236).

FIG. 31 shows representative x-ray diffraction patterns collected from cholesterol-enriched model membranes treated with vehicle (control), eicosapentaenoic acid (EPA), docosahexaenoic acid (DHA), EPA-DHA combination treatment, fenofibrate (Fenofib), nicotinic acid (Niacin), gemfibrozil (Gemfib), glycyrrhizin (Glyc), arachidonic acid (AA), α-linolenic acid (ALA), or docosapentaenoic acid (DPA).

FIG. 32 shows the comparative effects of EPA, DHA, EPA-DHA combination treatment, and fenofibrate on membrane cholesterol domain structural integrity.

FIG. 33 shows the comparative effects of EPA, DHA, EPA-DHA combination treatment, fenofibrate and glycyrrhizin (Glyc) on membrane cholesterol domain structural integrity.

FIG. 34 shows the comparative effects of EPA, DHA, EPA-DHA combination treatment, fenofibrate, glycyrrhizin (Glyc), and docosapentaenoic acid (DPA) on membrane cholesterol domain structural integrity.

FIG. 35 shows the comparative effects of α-linolenic acid (ALA), arachidonic acid (AA), eicosapentaenoic acid (EPA), docosapentaenoic acid (DPA), docosahexaenoic acid (DHA), and glycyrrhizin (Glyc) on membrane cholesterol domain structural integrity. Values are mean±SEM (N=3-6). *p<0.01 and **p<0.001 versus control; †p<0.05 and ‡p<0.001 versus DHA; § p<0.01 versus DPA (Student-Newman-Keuls multiple comparisons analysis; overall ANOVA: p<0.0001, F=12.021).

FIG. 36 shows the comparative effects of α-linolenic acid (ALA), arachidonic acid (AA), eicosapentaenoic acid (EPA), docosapentaenoic acid (DPA), docosahexaenoic acid (DHA), EPA-DHA combination treatment, and glycyrrhizin (Glyc) on membrane cholesterol domain structural integrity. Values are mean±SEM (N=3-6). *p<0.05 and **p<0.01 versus control; †p<0.01 vs. DHA; § p<0.05 versus DPA (Student-Newman-Keuls multiple comparisons analysis; overall ANOVA: p=0.0004, F=6.855).

FIG. 37 shows the dose-dependent disruptive effects of EPA on the structural integrity of pre-existing membrane cholesterol domains.

FIG. 38 shows sdLDL oxidation in the presence or absence of hyperglycemic conditions.

FIG. 39 depicts the reduction of glucose-induced sdLDL oxidation by EPA in a dose-dependent manner.

FIG. 40 shows comparative reductions in glucose-induced sdLDL oxidation by various agents.

FIG. 41 shows the increase in glucose-induced sdLDL oxidation with Vitamin E.

FIG. 42 shows the effects of oxidized low-density lipoprotein (oxLDL) on nitric oxide (NO) and peroxynitrite (ONOO⁻) release from human umbilical vein endothelial cells (HUVECs) pre-treated with eicosapentaenoic acid (EPA), atorvastatin o-hydroxy (active) metabolite (ATM), EPA/ATM combination, docosahexaenoic acid (DHA), fenofibrate (Fenofib), gemfibrozil (Gemfib), or nicotinic acid (Niacin).

FIG. 43 compares the effects of EPA, DHA, and EPA/ATM combination treatment NO and ONOO⁻ release from HUVECs treated concomitantly with oxLDL.

FIG. 44 compares the effects of EPA, DHA, and EPA/ATM combination treatment on NO and ONOO⁻ release from HUVECs treated concomitantly with native LDL (and then exposed to oxidative conditions) versus cells treated with oxLDL directly.

### DETAILED DESCRIPTION

While the present invention is capable of being embodied in various forms, the description below of several embodiments is made with the understanding that the present disclosure is to be considered as an exemplification of the invention, and is not intended to limit the invention to the specific embodiments illustrated. Headings are provided for convenience only and are not to be construed to limit the invention in any manner. Embodiments illustrated under any heading may be combined with embodiments illustrated under any other heading.

The use of numerical values in the various quantitative values specified in this application, unless expressly indicated otherwise, are stated as approximations as though the minimum and maximum values within the stated ranges were both preceded by the word “about.” Also, the disclosure of ranges is intended as a continuous range including every value between the minimum and maximum values recited as well as any ranges that can be formed by such values. Also disclosed herein are any and all ratios (and ranges of any such ratios) that can be formed by dividing a disclosed numeric value into any other disclosed numeric value. Accordingly, the skilled person will appreciate that many such ratios, ranges, and ranges of ratios can be unambiguously derived from the numerical values presented herein and in all instances such ratios, ranges, and ranges of ratios represent various embodiments of the present invention.

In one embodiment, the invention provides a method for treatment and/or prevention of a cardiovascular-related disease. The term “cardiovascular-related disease” herein refers to any disease or disorder of the heart or blood vessels (i.e. arteries and veins) or any symptom thereof. Non-limiting examples of cardiovascular-related disease and disorders include hypertriglyceridemia, hypercholesterolemia, mixed dyslipidemia, coronary heart disease, vascular disease, stroke, atherosclerosis, arrhythmia, hypertension, myocardial infarction, and other cardiovascular events.

The term “treatment” in relation a given disease or disorder, includes, but is not limited to, inhibiting the disease or disorder, for example, arresting the development of the disease or disorder; relieving the disease or disorder, for example, causing regression of the disease or disorder; or relieving a condition caused by or resulting from the disease or disorder, for example, relieving, preventing or treating symptoms of the disease or disorder. The term “prevention” in relation to a given disease or disorder means: preventing the onset of disease development if none had occurred, preventing the disease or disorder from occurring in a subject that may be predisposed to the disorder or disease but has not yet been diagnosed as having the disorder or disease, and/or preventing further disease/disorder development if already present.

In one embodiment, the present invention provides a method of reducing or preventing membrane cholesterol domain formation in a subject in need thereof, the method comprising administering to the subject a pharmaceutical composition comprising eicosapentaenoic acid or a derivative thereof. In one embodiment, the method comprises measuring membrane cholesterol domain formation in the subject prior to and/or after administering to the subject a pharmaceutical composition comprising eicosapentaenoic acid or a derivative thereof. In one embodiment, the method comprises a step of determining a reduction in or absence of an increase in cholesterol domain formation in the subject.

In another embodiment, the present invention provides a method of reducing or preventing oxidative modification of membrane polyunsaturated fatty acids in a subject in need thereof, the method comprising administering to the subject a pharmaceutical composition comprising eicosapentaenoic acid or a derivative thereof. In one embodiment, the method comprises comprising a step of measuring oxidative modification of membrane polyunsaturated fatty acids in the subject before and/or after administering to the subject the pharmaceutical composition comprising eicosapentaenoic acid or a derivative thereof. In one embodiment, the method comprises a step of determining a reduction in or absence of an increase in oxidative modification of membrane polyunsaturated fatty acids in the subject.

In one embodiment, the subject or subject group in need thereof has one or more of: hypercholesterolemia, familial hypercholesterolemia, high LDL-C serum levels, high total cholesterol levels, and/or low HDL-C serum levels.

In another embodiment, the subject or subject group being treated has a baseline triglyceride level (or median baseline triglyceride level in the case of a subject group), fed or fasting, of at least about 300 mg/dl, at least about 400 mg/dl, at least about 500 mg/dl, at least about 600 mg/dl, at least about 700 mg/dl, at least about 800 mg/dl, at least about 900 mg/dl, at least about 1000 mg/dl, at least about 1100 mg/dl, at least about 1200 mg/dl, at least about 1300 mg/dl, at least about 1400 mg/dl, or at least about 1500 mg/dl, for example about 400 mg/dl to about 2500 mg/dl, about 450 mg/dl to about 2000 mg/dl or about 500 mg/dl to about 1500 mg/dl.

In one embodiment, the subject or subject group being treated in accordance with methods of the invention has previously been treated with Lovaza® and has experienced an increase in, or no decrease in, LDL-C levels and/or non-HDL-C levels. In one such embodiment, Lovaza® therapy is discontinued and replaced by a method of the present invention.

In another embodiment, the subject or subject group being treated in accordance with methods of the invention exhibits a fasting baseline absolute plasma level of free EPA (or mean thereof in the case of a subject group) not greater than about 0.70 nmol/ml, not greater than about 0.65 nmol/ml, not greater than about 0.60 nmol/ml, not greater than about 0.55 nmol/ml, not greater than about 0.50 nmol/ml, not greater than about 0.45 nmol/ml, or not greater than about 0.40 nmol/ml. In another embodiment, the subject or subject group being treated in accordance with methods of the invention exhibits a baseline fasting plasma level (or mean thereof) of free EPA, expressed as a percentage of total free fatty acid, of not more than about 3%, not more than about 2.5%, not more than about 2%, not more than about 1.5%, not more than about 1%, not more than about 0.75%, not more than about 0.5%, not more than about 0.25%, not more than about 0.2% or not more than about 0.15%. In one such embodiment, free plasma EPA and/or total fatty acid levels are determined prior to initiating therapy.

In another embodiment, the subject or subject group being treated in accordance with methods of the invention exhibits a fasting baseline absolute plasma level of total fatty acid (or mean thereof) not greater than about 250 nmol/ml, not greater than about 200 nmol/ml, not greater than about 150 nmol/ml, not greater than about 100 nmol/ml, or not greater than about 50 nmol/ml.

In another embodiment, the subject or subject group being treated in accordance with methods of the invention exhibits a fasting baseline plasma, serum or red blood cell membrane EPA level not greater than about 70 μg/ml, not greater than about 60 μg/ml, not greater than about 50 μg/ml, not greater than about 40 μg/ml, not greater than about 30 μg/ml, or not greater than about 25 μg/ml.

In another embodiment, methods of the present invention comprise a step of measuring the subject's (or subject group's mean) baseline lipid profile prior to initiating therapy. In another embodiment, methods of the invention comprise the step of identifying a subject or subject group having one or more of the following: baseline non-HDL-C value of about 200 mg/dl to about 400 mg/dl, for example at least about 210 mg/dl, at least about 220 mg/dl, at least about 230 mg/dl, at least about 240 mg/dl, at least about 250 mg/dl, at least about 260 mg/dl, at least about 270 mg/dl, at least about 280 mg/dl, at least about 290 mg/dl, or at least about 300 mg/dl; baseline total cholesterol value of about 250 mg/dl to about 400 mg/dl, for example at least about 260 mg/dl, at least about 270 mg/dl, at least about 280 mg/dl or at least about 290 mg/dl; baseline vLDL-C value of about 140 mg/dl to about 200 mg/dl, for example at least about 150 mg/dl, at least about 160 mg/dl, at least about 170 mg/dl, at least about 180 mg/dl or at least about 190 mg/dl; baseline HDL-C value of about 10 to about 60 mg/dl, for example not more than about 40 mg/dl, not more than about 35 mg/dl, not more than about 30 mg/dl, not more than about 25 mg/dl, not more than about 20 mg/dl, or not more than about 15 mg/dl; and/or baseline LDL-C value of about 50 to about 300 mg/dl, for example not less than about 100 mg/dl, not less than about 90 mg/dl, not less than about 80 mg/dl, not less than about 70 mg/dl, not less than about 60 mg/dl or not less than about 50 mg/dl.

In a related embodiment, upon treatment in accordance with the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits one or more of the following outcomes:

(a) reduced triglyceride levels compared to baseline or control;

(b) reduced Apo B levels compared to baseline or control;

(c) increased HDL-C levels compared to baseline or control;

(d) no increase in LDL-C levels compared to baseline or control;

(e) a reduction in LDL-C levels compared to baseline or control;

(f) a reduction in non-HDL-C levels compared to baseline or control;

(g) a reduction in VLDL levels compared to baseline or control;

(h) an increase in apo A-I levels compared to baseline or control;

(i) an increase in apo A-I/apo B ratio compared to baseline or control;

(j) a reduction in lipoprotein A levels compared to baseline or control;

(k) a reduction in LDL particle number compared to baseline or control;

(l) an increase in LDL size compared to baseline or control;

(m) a reduction in remnant-like particle cholesterol compared to baseline or control;

(n) a reduction in oxidized LDL compared to baseline or control;

(o) no change or a reduction in fasting plasma glucose (FPG) compared to baseline or control;

(p) a reduction in hemoglobin A_{1c} (HbA_{1c}) compared to baseline or control;

(q) a reduction in homeostasis model insulin resistance compared to baseline or control;

(r) a reduction in lipoprotein associated phospholipase A2 compared to baseline or control;

(s) a reduction in intracellular adhesion molecule-1 compared to baseline or control;

(t) a reduction in interleukin-6 compared to baseline or control;

(u) a reduction in plasminogen activator inhibitor-1 compared to baseline or control;

(v) a reduction in high sensitivity C-reactive protein (hsCRP) compared to baseline or control;

(w) an increase in serum or plasma EPA compared to baseline or control;

(x) an increase in red blood cell (RBC) membrane EPA compared to baseline or control;

(y) a reduction or increase in one or more of serum phospholipid and/or red blood cell content of docosahexaenoic acid (DHA), docosapentaenoic acid (DPA), arachidonic acid (AA), palmitic acid (PA), stearidonic acid (SA) or oleic acid (OA) compared to baseline or control;

(z) a reduction in or prevention of membrane cholesterol domain formation compared to baseline or control; and/or

(aa) a reduction in or prevention of oxidative modification of membrane polyunsaturated fatty acids compared to baseline or control; and or

(bb) no increase, no substantial increase, or a reduction in oxidized sdLDL compared to baseline or control.

In one embodiment, upon administering a composition of the invention to a subject, the subject exhibits a decrease in triglyceride levels, an increase in the concentrations of EPA and DPA (n−3) in red blood cells, and an increase of the ratio of EPA:arachidonic acid in red blood cells. In a related embodiment the subject exhibits substantially no or no increase in RBC DHA.

In one embodiment, methods of the present invention comprise measuring baseline levels of one or more markers set forth in (a)-(bb) above prior to dosing the subject or subject group. In another embodiment, the methods comprise administering a composition as disclosed herein to the subject after baseline levels of one or more markers set forth in (a)-(bb) are determined, and subsequently taking an additional measurement of said one or more markers.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, any 25 or more of, any 26 or more of, any 27 or more of, or all 28 of outcomes (a)-(bb) described immediately above.

In another embodiment, upon treatment with a composition of the present invention, the subject or subject group exhibits one or more of the following outcomes:

(a) a reduction in triglyceride level of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(b) a less than 30% increase, less than 20% increase, less than 10% increase, less than 5% increase or no increase in non-HDL-C levels or a reduction in non-HDL-C levels of at least about 1%, at least about 3%, at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(c) substantially no change in HDL-C levels, no change in HDL-C levels, or an increase in HDL-C levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(d) a less than 60% increase, a less than 50% increase, a less than 40% increase, a less than 30% increase, less than 20% increase, less than 10% increase, less than 5% increase or no increase in LDL-C levels or a reduction in LDL-C levels of at least about 5%, at least about 10%, at least about 15%, at least about 20%, at least about 25%, at least about 30%, at least about 35%, at least about 40%, at least about 45%, at least about 50%, at least about 55%, at least about 55% or at least about 75% (actual % change or median % change) as compared to baseline;

(e) a decrease in Apo B levels of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) as compared to baseline;

(f) a reduction in vLDL levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(g) an increase in apo A-I levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(h) an increase in apo A-I/apo B ratio of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(i) a reduction in lipoprotein (a) levels of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(j) a reduction in mean LDL particle number of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(k) an increase in mean LDL particle size of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(l) a reduction in remnant-like particle cholesterol of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(m) a reduction in oxidized LDL of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(n) substantially no change, no significant change, or a reduction (e.g. in the case of a diabetic subject) in fasting plasma glucose (FPG) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(o) substantially no change, no significant change or a reduction in hemoglobin A_{1c} (HbA_{1c}) of at least about 5%, at least about 10%, at least about 15%, at least about 20%, at least about 25%, at least about 30%, at least about 35%, at least about 40%, at least about 45%, or at least about 50% (actual % change or median % change) compared to baseline;

(p) a reduction in homeostasis model index insulin resistance of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(q) a reduction in lipoprotein associated phospholipase A2 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(r) a reduction in intracellular adhesion molecule-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(s) a reduction in interleukin-6 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(t) a reduction in plasminogen activator inhibitor-1 of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(u) a reduction in high sensitivity C-reactive protein (hsCRP) of at least about 5%, at least about 10%, at least about 15%, at least about 20%, at least about 25%, at least about 30%, at least about 35%, at least about 40%, at least about 45%, at least about 50%, or at least about 100% (actual % change or median % change) compared to baseline;

(v) an increase in serum, plasma and/or RBC EPA of at least about 5%, at least about 10%, at least about 15%, at least about 20%, at least about 25%, at least about 30%, at least about 35%, at least about 40%, at least about 45%, at least about 50%, at least about 100%, at least about 200% or at least about 400% (actual % change or median % change) compared to baseline;

(w) an increase in serum phospholipid and/or red blood cell membrane EPA of at least about 5%, at least about 10%, at least about 15%, at least about 20%, at least about 25%, at least about 30%, at least about 35%, at least about 40%, at least about 45%, at least about 50%, at least about 100%, at least about 200%, or at least about 400% (actual % change or median % change) compared to baseline;

(x) a reduction or increase in one or more of serum phospholipid and/or red blood cell DHA, DPA, AA, PA and/or OA of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline;

(y) a reduction in total cholesterol of at least about 5%, at least about 10%, at least about 15%, at least about 20%, at least about 25%, at least about 30%, at least about 35%, at least about 40%, at least about 45%, at least about 50%, at least about 55% or at least about 75% (actual % change or median % change) compared to baseline;

(z) a reduction in membrane cholesterol domain formation of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or about 100% (actual % change or median % change) compared to baseline or control;

(aa) a reduction in oxidative modification of membrane polyunsaturated fatty acids of at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or about 100% (actual % change or median % change) compared to baseline or control; and/or

(bb) no increase, no substantial increase, or a reduction in oxidized sdLDL of at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or greater than 95% (actual % change or median % change) compared to baseline or control.

In one embodiment, methods of the present invention comprise measuring baseline levels of one or more markers set forth in (a)-(bb) prior to dosing the subject or subject group. In another embodiment, the methods comprise administering a composition as disclosed herein to the subject after baseline levels of one or more markers set forth in (a)-(bb) are determined, and subsequently taking a second measurement of the one or more markers as measured at baseline for comparison thereto.

In another embodiment, upon treatment with a composition of the present invention, for example over a period of about 1 to about 200 weeks, about 1 to about 100 weeks, about 1 to about 80 weeks, about 1 to about 50 weeks, about 1 to about 40 weeks, about 1 to about 20 weeks, about 1 to about 15 weeks, about 1 to about 12 weeks, about 1 to about 10 weeks, about 1 to about 5 weeks, about 1 to about 2 weeks or about 1 week, the subject or subject group exhibits any 2 or more of, any 3 or more of, any 4 or more of, any 5 or more of, any 6 or more of, any 7 or more of, any 8 or more of, any 9 or more of, any 10 or more of, any 11 or more of, any 12 or more of, any 13 or more of, any 14 or more of, any 15 or more of, any 16 or more of, any 17 or more of, any 18 or more of, any 19 or more of, any 20 or more of, any 21 or more of, any 22 or more of, any 23 or more of, any 24 or more of, any 25 or more of, any 26 or more of, any 27 or more of, or all 28 of outcomes (a)-(bb) described immediately above.

Parameters (a)-(y) can be measured in accordance with any clinically acceptable methodology. For example, triglycerides, total cholesterol, HDL-C and fasting blood sugar can be sample from serum and analyzed using standard photometry techniques. VLDL-TG, LDL-C and VLDL-C can be calculated or determined using serum lipoprotein fractionation by preparative ultracentrifugation and subsequent quantitative analysis by refractometry or by analytic ultracentrifugal methodology. Apo A1, Apo B and hsCRP can be determined from serum using standard nephelometry techniques. Lipoprotein (a) can be determined from serum using standard turbidimetric immunoassay techniques. LDL particle number and particle size can be determined using nuclear magnetic resonance (NMR) spectrometry. Remnant lipoproteins and LDL-phospholipase A2 can be determined from EDTA plasma or serum and serum, respectively, using enzymatic immunoseparation techniques. Oxidized LDL, intercellular adhesion molecule-1 and interleukin-6 levels can be determined from serum using standard enzyme immunoassay techniques. These techniques are described in detail in standard textbooks, for example Tietz Fundamentals of Clinical Chemistry, 6^{th} Ed. (Burtis, Ashwood and Borter Eds.), WB Saunders Company. Parameters (z) to (bb) can be measured in accordance with any clinically acceptable methodology or can be estimated by any suitable in vitro experiment, for example, one similar to that described in Examples 3-7.

In one embodiment, subjects fast for up to 12 hours prior to blood sample collection, for example about 10 hours.

In another embodiment, the present invention provides a method of treating or preventing primary hypercholesterolemia and/or mixed dyslipidemia (Fredrickson Types IIa and IIb) in a patient in need thereof, comprising administering to the patient one or more compositions as disclosed herein. In a related embodiment, the present invention provides a method of reducing triglyceride levels in a subject or subjects when treatment with a statin or niacin extended-release monotherapy is considered inadequate (Frederickson type IV hyperlipidemia).

In another embodiment, the present invention provides a method of treating or preventing risk of recurrent nonfatal myocardial infarction in a patient with a history of myocardial infarction, comprising administering to the patient one or more compositions as disclosed herein.

In another embodiment, the present invention provides a method of slowing progression of or promoting regression of atherosclerotic disease in a patient in need thereof, comprising administering to a subject in need thereof one or more compositions as disclosed herein.

In another embodiment, the present invention provides a method of treating or preventing very high serum triglyceride levels (e.g. Types IV and V hyperlipidemia) in a patient in need thereof, comprising administering to the patient one or more compositions as disclosed herein.

In another embodiment, the present invention provides a method of treating subjects having very high serum triglyceride levels (e.g. greater than 1000 mg/dl or greater than 2000 mg/dl) and that are at risk of developing pancreatitis, comprising administering to the patient one or more compositions as disclosed herein.

In one embodiment, a composition of the invention is administered to a subject in an amount sufficient to provide a daily dose of eicosapentaenoic acid of about 1 mg to about 10,000 mg, 25 about 5000 mg, about 50 to about 3000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, about 5000 mg, about 5025 mg, about 5050 mg, about 5075 mg, about 5100 mg, about 5125 mg, about 5150 mg, about 5175 mg, about 5200 mg, about 5225 mg, about 5250 mg, about 5275 mg, about 5300 mg, about 5325 mg, about 5350 mg, about 5375 mg, about 5400 mg, about 5425 mg, about 5450 mg, about 5475 mg, about 5500 mg, about 5525 mg, about 5550 mg, about 5575 mg, about 5600 mg, about 5625 mg, about 5650 mg, about 5675 mg, about 5700 mg, about 5725 mg, about 5750 mg, about 5775 mg, about 5800 mg, about 5825 mg, about 5850 mg, about 5875 mg, about 5900 mg, about 5925 mg, about 5950 mg, about 5975 mg, about 6000 mg, about 6025 mg, about 6050 mg, about 6075 mg, about 6100 mg, about 6125 mg, about 6150 mg, about 6175 mg, about 6200 mg, about 6225 mg, about 6250 mg, about 6275 mg, about 6300 mg, about 6325 mg, about 6350 mg, about 6375 mg, about 6400 mg, about 6425 mg, about 6450 mg, about 6475 mg, about 6500 mg, about 6525 mg, about 6550 mg, about 6575 mg, about 6600 mg, about 6625 mg, about 6650 mg, about 6675 mg, about 6700 mg, about 6725 mg, about 6750 mg, about 6775 mg, about 6800 mg, about 6825 mg, about 6850 mg, about 6875 mg, about 6900 mg, about 6925 mg, about 6950 mg, about 6975 mg, about 7000 mg, about 7025 mg, about 7050 mg, about 7075 mg, about 7100 mg, about 7125 mg, about 7150 mg, about 7175 mg, about 7200 mg, about 7225 mg, about 7250 mg, about 7275 mg, about 7300 mg, about 7325 mg, about 7350 mg, about 7375 mg, about 7400 mg, about 7425 mg, about 7450 mg, about 7475 mg, about 7500 mg, about 7525 mg, about 7550 mg, about 7575 mg, about 7600 mg, about 7625 mg, about 7650 mg, about 7675 mg, about 7700 mg, about 7725 mg, about 7750 mg, about 7775 mg, about 7800 mg, about 7825 mg, about 7850 mg, about 7875 mg, about 7900 mg, about 7925 mg, about 7950 mg, about 7975 mg, about 8000 mg, about 8025 mg, about 8050 mg, about 8075 mg, about 8100 mg, about 8125 mg, about 8150 mg, about 8175 mg, about 8200 mg, about 8225 mg, about 8250 mg, about 8275 mg, about 8300 mg, about 8325 mg, about 8350 mg, about 8375 mg, about 8400 mg, about 8425 mg, about 8450 mg, about 8475 mg, about 8500 mg, about 8525 mg, about 8550 mg, about 8575 mg, about 8600 mg, about 8625 mg, about 8650 mg, about 8675 mg, about 8700 mg, about 8725 mg, about 8750 mg, about 8775 mg, about 8800 mg, about 8825 mg, about 8850 mg, about 8875 mg, about 8900 mg, about 8925 mg, about 8950 mg, about 8975 mg, about 9000 mg, about 9025 mg, about 9050 mg, about 9075 mg, about 9100 mg, about 9125 mg, about 9150 mg, about 9175 mg, about 9200 mg, about 9225 mg, about 9250 mg, about 9275 mg, about 9300 mg, about 9325 mg, about 9350 mg, about 9375 mg, about 9400 mg, about 9425 mg, about 9450 mg, about 9475 mg, about 9500 mg, about 9525 mg, about 9550 mg, about 9575 mg, about 9600 mg, about 9625 mg, about 9650 mg, about 9675 mg, about 9700 mg, about 9725 mg, about 9750 mg, about 9775 mg, about 9800 mg, about 9825 mg, about 9850 mg, about 9875 mg, about 9900 mg, about 9925 mg, about 9950 mg, about 9975 mg, or about 10,000 mg.

In another embodiment, any of the methods disclosed herein are used in treatment or prevention of a subject or subjects that consume a traditional Western diet. In one embodiment, the methods of the invention include a step of identifying a subject as a Western diet consumer or prudent diet consumer and then treating the subject if the subject is deemed a Western diet consumer. The term “Western diet” herein refers generally to a typical diet consisting of, by percentage of total calories, about 45% to about 50% carbohydrate, about 35% to about 40% fat, and about 10% to about 15% protein. A Western diet may alternately or additionally be characterized by relatively high intakes of red and processed meats, sweets, refined grains, and desserts, for example more than 50%, more than 60% or more or 70% of total calories come from these sources.

In one embodiment, a composition for use in methods of the invention comprises eicosapentaenoic acid, or a pharmaceutically acceptable ester, derivative, conjugate or salt thereof, or mixtures of any of the foregoing, collectively referred to herein as “EPA.” The term “pharmaceutically acceptable” in the present context means that the substance in question does not produce unacceptable toxicity to the subject or interaction with other components of the composition.

In one embodiment, the EPA comprises all-cis eicosa-5,8,11,14,17-pentaenoic acid. In another embodiment, the EPA comprises an eicosapentaenoic acid ester. In another embodiment, the EPA comprises a C₁-C₅ alkyl ester of eicosapentaenoic acid. In another embodiment, the EPA comprises eicosapentaenoic acid ethyl ester, eicosapentaenoic acid methyl ester, eicosapentaenoic acid propyl ester, or eicosapentaenoic acid butyl ester. In another embodiment, the EPA comprises In one embodiment, the EPA comprises all-cis eicosa-5,8,11,14,17-pentaenoic acid ethyl ester.

In another embodiment, the EPA is in the form of ethyl-EPA, lithium EPA, mono-, di- or triglyceride EPA or any other ester or salt of EPA, or the free acid form of EPA. The EPA may also be in the form of a 2-substituted derivative or other derivative which slows down its rate of oxidation but does not otherwise change its biological action to any substantial degree.

In another embodiment, EPA is present in a composition useful in accordance with methods of the invention in an amount of about 50 mg to about 5000 mg, about 75 mg to about 2500 mg, or about 100 mg to about 1000 mg, for example about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, about 400 mg, about 425 mg, about 450 mg, about 475 mg, about 500 mg, about 525 mg, about 550 mg, about 575 mg, about 600 mg, about 625 mg, about 650 mg, about 675 mg, about 700 mg, about 725 mg, about 750 mg, about 775 mg, about 800 mg, about 825 mg, about 850 mg, about 875 mg, about 900 mg, about 925 mg, about 950 mg, about 975 mg, about 1000 mg, about 1025 mg, about 1050 mg, about 1075 mg, about 1100 mg, about 1025 mg, about 1050 mg, about 1075 mg, about 1200 mg, about 1225 mg, about 1250 mg, about 1275 mg, about 1300 mg, about 1325 mg, about 1350 mg, about 1375 mg, about 1400 mg, about 1425 mg, about 1450 mg, about 1475 mg, about 1500 mg, about 1525 mg, about 1550 mg, about 1575 mg, about 1600 mg, about 1625 mg, about 1650 mg, about 1675 mg, about 1700 mg, about 1725 mg, about 1750 mg, about 1775 mg, about 1800 mg, about 1825 mg, about 1850 mg, about 1875 mg, about 1900 mg, about 1925 mg, about 1950 mg, about 1975 mg, about 2000 mg, about 2025 mg, about 2050 mg, about 2075 mg, about 2100 mg, about 2125 mg, about 2150 mg, about 2175 mg, about 2200 mg, about 2225 mg, about 2250 mg, about 2275 mg, about 2300 mg, about 2325 mg, about 2350 mg, about 2375 mg, about 2400 mg, about 2425 mg, about 2450 mg, about 2475 mg, about 2500 mg, about 2525 mg, about 2550 mg, about 2575 mg, about 2600 mg, about 2625 mg, about 2650 mg, about 2675 mg, about 2700 mg, about 2725 mg, about 2750 mg, about 2775 mg, about 2800 mg, about 2825 mg, about 2850 mg, about 2875 mg, about 2900 mg, about 2925 mg, about 2950 mg, about 2975 mg, about 3000 mg, about 3025 mg, about 3050 mg, about 3075 mg, about 3100 mg, about 3125 mg, about 3150 mg, about 3175 mg, about 3200 mg, about 3225 mg, about 3250 mg, about 3275 mg, about 3300 mg, about 3325 mg, about 3350 mg, about 3375 mg, about 3400 mg, about 3425 mg, about 3450 mg, about 3475 mg, about 3500 mg, about 3525 mg, about 3550 mg, about 3575 mg, about 3600 mg, about 3625 mg, about 3650 mg, about 3675 mg, about 3700 mg, about 3725 mg, about 3750 mg, about 3775 mg, about 3800 mg, about 3825 mg, about 3850 mg, about 3875 mg, about 3900 mg, about 3925 mg, about 3950 mg, about 3975 mg, about 4000 mg, about 4025 mg, about 4050 mg, about 4075 mg, about 4100 mg, about 4125 mg, about 4150 mg, about 4175 mg, about 4200 mg, about 4225 mg, about 4250 mg, about 4275 mg, about 4300 mg, about 4325 mg, about 4350 mg, about 4375 mg, about 4400 mg, about 4425 mg, about 4450 mg, about 4475 mg, about 4500 mg, about 4525 mg, about 4550 mg, about 4575 mg, about 4600 mg, about 4625 mg, about 4650 mg, about 4675 mg, about 4700 mg, about 4725 mg, about 4750 mg, about 4775 mg, about 4800 mg, about 4825 mg, about 4850 mg, about 4875 mg, about 4900 mg, about 4925 mg, about 4950 mg, about 4975 mg, or about 5000 mg

In another embodiment, a composition useful in accordance with the invention contains not more than about 10%, not more than about 9%, not more than about 8%, not more than about 7%, not more than about 6%, not more than about 5%, not more than about 4%, not more than about 3%, not more than about 2%, not more than about 1%, or not more than about 0.5%, by weight of all fatty acids (and/or derivatives thereof) present, docosahexaenoic acid (DHA), if any. In another embodiment, a composition of the invention contains substantially no docosahexaenoic acid. In still another embodiment, a composition useful in the present invention contains no docosahexaenoic acid and/or derivative thereof.

In another embodiment, EPA comprises at least 70%, at least 80%, at least 90%, at least 95%, at least 96%, at least 97%, at least 98%, at least 99%, or 100%, by weight of all fatty acids (and/or derivatives thereof) present, in a composition that is useful in methods of the present invention.

In one embodiment, a composition of the invention comprises ultra-pure EPA. The term “ultra-pure” as used herein with respect to EPA refers to a composition comprising at least 95%, by weight of all fatty acids (and/or derivatives thereof) present, EPA (as the term “EPA” is defined and exemplified herein). Ultra-pure EPA comprises at least 96%, by weight of all fatty acids (and/or derivatives thereof) present, EPA, at least 97%, by weight of all fatty acids (and/or derivatives thereof) present, EPA, or at least 98%, by weight of all fatty acids (and/or derivatives thereof) present, EPA, wherein the EPA is any form of EPA as set forth herein.

In another embodiment, a composition useful in accordance with methods of the invention contains less than 10%, less than 9%, less than 8%, less than 7%, less than 6%, less than 5%, less than 4%, less than 3%, less than 2%, less than 1%, less than 0.5% or less than 0.25%, by weight of all fatty acids (and/or derivatives thereof) present, of any fatty acid other than EPA. Illustrative examples of a “fatty acid other than EPA” include linolenic acid (LA), arachidonic acid (AA), docosahexaenoic acid (DHA), alpha-linolenic acid (ALA), stearidonic acid (STA), eicosatrienoic acid (ETA) and/or docosapentaenoic acid (DPA). In another embodiment, a composition useful in accordance with methods of the invention contains about 0.1% to about 4%, about 0.5% to about 3%, or about 1% to about 2%, by weight of all fatty acids (and/or derivatives thereof) present, other than EPA and/or DHA.

In another embodiment, a composition useful in accordance with the invention has one or more of the following features: (a) eicosapentaenoic acid ethyl ester represents at least about 96%, at least about 97%, or at least about 98%, by weight of all fatty acids (and/or derivatives thereof) present, in the composition; (b) the composition contains not more than about 4%, not more than about 3%, or not more than about 2%, by weight of all fatty acids (and/or derivatives thereof) present, other than eicosapentaenoic acid ethyl ester; (c) the composition contains not more than about 0.6%, not more than about 0.5%, or not more than about 0.4%, by weight of all fatty acids (and/or derivatives thereof) present, of any individual fatty acid other than eicosapentaenoic acid ethyl ester; (d) the composition has a refractive index (20° C.) of about 1 to about 2, about 1.2 to about 1.8 or about 1.4 to about 1.5; (e) the composition has a specific gravity (20° C.) of about 0.8 to about 1.0, about 0.85 to about 0.95 or about 0.9 to about 0.92; (e) the composition contains not more than about 20 ppm, not more than about 15 ppm or not more than about 10 ppm heavy metals, (f) the composition contains not more than about 5 ppm, not more than about 4 ppm, not more than about 3 ppm, or not more than about 2 ppm arsenic, and/or (g) the composition has a peroxide value of not more than about 5 meq/kg, not more than about 4 meq/kg, not more than about 3 meq/kg, or not more than about 2 meq/kg.

In another embodiment, a composition useful in accordance with the invention comprises, consists of or consists essentially of at least 95%, by weight of all fatty acids (and/or derivatives thereof) present, ethyl eicosapentaenoate (EPA-E), about 0.2% to about 0.5%, by weight of all fatty acids (and/or derivatives thereof) present, ethyl octadecatetraenoate (ODTA-E), about 0.05% to about 0.25%, by weight of all fatty acids (and/or derivatives thereof) present, ethyl nonadecapentaenoate (NDPA-E), about 0.2% to about 0.45%, by weight of all fatty acids (and/or derivatives thereof) present, ethyl arachidonate (AA-E), about 0.3% to about 0.5%, by weight of all fatty acids (and/or derivatives thereof) present, ethyl eicosatetraenoate (ETA-E), and about 0.05% to about 0.32%, by weight of all fatty acids (and/or derivatives thereof) present, ethyl heneicosapentaenoate (HPA-E). In another embodiment, the composition is present in a capsule shell.

In another embodiment, compositions useful in accordance with the invention comprise, consist essential of, or consist of at least 95%, 96% or 97%, by weight of all fatty acids (and/or derivatives thereof) present, ethyl eicosapentaenoate, about 0.2% to about 0.5% by weight ethyl octadecatetraenoate, about 0.05% to about 0.25%, by weight of all fatty acids (and/or derivatives thereof) present, ethyl nonadecapentaenoate, about 0.2% to about 0.45%, by weight of all fatty acids (and/or derivatives thereof) present, ethyl arachidonate, about 0.3% to about 0.5%, by weight of all fatty acids (and/or derivatives thereof) present, ethyl eicosatetraenoate, and about 0.05% to about 0.32%, by weight of all fatty acids (and/or derivatives thereof) present, ethyl heneicosapentaenoate. Optionally, the composition contains not more than about 0.06%, about 0.05%, or about 0.04%, by weight of all fatty acids (and/or derivatives thereof) present, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, about 500 mg to about 1 g of the composition is provided in a capsule shell.

In another embodiment, compositions useful in accordance with the invention comprise, consist essential of, or consist of at least 96%, by weight of all fatty acids (and/or derivatives thereof) present, ethyl eicosapentaenoate, about 0.22% to about 0.4%, by weight of all fatty acids (and/or derivatives thereof) present, ethyl octadecatetraenoate, about 0.075% to about 0.20%, by weight of all fatty acids (and/or derivatives thereof) present, ethyl nonadecapentaenoate, about 0.25% to about 0.40%, by weight of all fatty acids (and/or derivatives thereof) present, ethyl arachidonate, about 0.3% to about 0.4%, by weight of all fatty acids (and/or derivatives thereof) present, ethyl eicosatetraenoate and about 0.075% to about 0.25%, by weight of all fatty acids (and/or derivatives thereof) present, ethyl heneicosapentaenoate. Optionally, the composition contains not more than about 0.06%, about 0.05%, or about 0.04%, by weight of all fatty acids (and/or derivatives thereof) present, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell. In one embodiment, the dosage form is a gel or liquid capsule and is packaged in blister packages of about 1 to about 20 capsules per sheet.

In another embodiment, compositions useful in accordance with the invention comprise, consist essential of, or consist of at least 96%, 97% or 98%, by weight of all fatty acids (and/or derivatives thereof) present, ethyl eicosapentaenoate, about 0.25% to about 0.38%, by weight of all fatty acids (and/or derivatives thereof) present, ethyl octadecatetraenoate, about 0.10% to about 0.15%, by weight of all fatty acids (and/or derivatives thereof) present, ethyl nonadecapentaenoate, about 0.25% to about 0.35%, by weight of all fatty acids (and/or derivatives thereof) present, ethyl arachidonate, about 0.31% to about 0.38%, by weight of all fatty acids (and/or derivatives thereof) present, ethyl eicosatetraenoate, and about 0.08% to about 0.20%, by weight of all fatty acids (and/or derivatives thereof) present, ethyl heneicosapentaenoate. Optionally, the composition contains not more than about 0.06%, about 0.05%, or about 0.04%, by weight of all fatty acids (and/or derivatives thereof) present, DHA or derivative thereof such as ethyl-DHA. In one embodiment the composition contains substantially no or no amount of DHA or derivative thereof such as ethyl-DHA. The composition further optionally comprises one or more antioxidants (e.g. tocopherol) or other impurities in an amount of not more than about 0.5% or not more than 0.05%. In another embodiment, the composition comprises about 0.05% to about 0.4%, for example about 0.2% by weight tocopherol. In another embodiment, the invention provides a dosage form comprising about 500 mg to about 1 g of the foregoing composition in a capsule shell.

In another embodiment, a composition as described herein is administered to a subject once or twice per day. In another embodiment, 1, 2, 3 or 4 capsules, each containing about 1 g of a composition as described herein, are administered to a subject daily. In another embodiment, 1 or 2 capsules, each containing about 1 g of a composition as described herein, are administered to the subject in the morning, for example between about 5 am and about 11 am, and 1 or 2 capsules, each containing about 1 g of a composition as described herein, are administered to the subject in the evening, for example between about 5 pm and about 11 pm.

In one embodiment, a subject being treated in accordance with methods of the invention is not otherwise on lipid-altering therapy, for example statin, fibrate, niacin and/or ezetimibe therapy.

In another embodiment, compositions useful in accordance with methods of the invention are orally deliverable. The terms “orally deliverable” or “oral administration” herein include any form of delivery of a therapeutic agent or a composition thereof to a subject wherein the agent or composition is placed in the mouth of the subject, whether or not the agent or composition is swallowed. Thus “oral administration” includes buccal and sublingual as well as esophageal administration. In one embodiment, the composition is present in a capsule, for example a soft gelatin capsule.

A composition for use in accordance with the invention can be formulated as one or more dosage units. The terms “dose unit” and “dosage unit” herein refer to a portion of a pharmaceutical composition that contains an amount of a therapeutic agent suitable for a single administration to provide a therapeutic effect. Such dosage units may be administered one to a plurality (i.e. 1 to about 10, 1 to 8, 1 to 6, 1 to 4 or 1 to 2) of times per day, or as many times as needed to elicit a therapeutic response.

In another embodiment, the invention provides use of any composition described herein for treating moderate to severe hypertriglyceridemia in a subject in need thereof, comprising: providing a subject having a fasting baseline triglyceride level of 500 mg/dl to about 1500 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid or derivative thereof. In some embodiments, cholesterol domain formation in membranes of the subject is reduced or prevented. In some embodiments, the subject experiences no substantial increase, or no increase, or a reduction, in LDL-C levels.

In another embodiment, the invention provides use of any composition described herein for treating moderate to severe hypertriglyceridemia in a subject in need thereof, comprising: providing a subject on statin therapy and having a fasting baseline triglyceride level of about 200 mg/dl to 499 mg/dl and administering to the subject a pharmaceutical composition as described herein. In one embodiment, the composition comprises about 1 g to about 4 g of eicosapentaenoic acid ethyl ester, wherein the composition contains substantially no docosahexaenoic acid. In some embodiments, cholesterol domain formation in membranes of the subject is reduced or prevented. In some embodiments, the subject experiences no substantial increase, or no increase, or a reduction, in LDL-C levels.

In one embodiment, compositions of the invention, upon storage in a closed container maintained at room temperature, refrigerated (e.g. about 5 to about 5-10° C.) temperature, or frozen for a period of about 1, 2, 3, 4, 5, 6, 7, 8, 9, 10, 11, or 12 months, exhibit at least about 90%, at least about 95%, at least about 97.5%, or at least about 99% of the active ingredient(s) originally present therein.

In one embodiment, the invention provides use of a composition as described herein in manufacture of a medicament for treatment of any of a cardiovascular-related disease. In another embodiment, the subject is diabetic.

In one embodiment, a composition as set forth herein is packaged together with instructions for using the composition to treat a cardiovascular disorder.

### EXAMPLES

### Example 1

A multi-center, placebo-controlled randomized, double-blind, 12-week study with an open-label extension was performed to evaluate the efficacy and safety of AMR101 in patients with fasting triglyceride levels ≥500 mg/dL. The primary objective of the study was to determine the efficacy of AMR101 2 g daily and 4 g daily, compared to placebo, in lowering fasting TG levels in patients with fasting TG levels ≥500 mg/dL and ≤1500 mg/dL (≥5.65 mmol/L and ≤16.94 mmol/L).

The secondary objectives of this study were the following:
1. To determine the safety and tolerability of AMR101 2 g daily and 4 g daily;
2. To determine the effect of AMR101 on lipid and apolipoprotein profiles;
3. To determine the effect of AMR101 on low-density lipoprotein (LDL) particle number and size;
4. To determine the effect of AMR101 on oxidized LDL;
5. To determine the effect of AMR101 on fasting plasma glucose (FPG) and hemoglobin A_{1c} (HbA_{1c});
6. To determine the effect of AMR101 on insulin resistance;
7. To determine the effect of AMR101 on high-sensitivity C-reactive protein (hsCRP);
8. To determine the effects of AMR101 2 g daily and 4 g daily on the incorporation of fatty acids into red blood cell membranes and into plasma phospholipids;
9. To explore the relationship between baseline fasting TG levels and the reduction in fasting TG levels; and
10. To explore the relationship between an increase in red blood cell membrane eicosapentaenoic acid (EPA) concentrations and the reduction in fasting TG levels.

The population for this study was men and women (women of childbearing potential needed to be on contraception or practice abstinence) >18 years of age with a body mass index ≤45 kg/m² who were not on lipid-altering therapy or were not currently on lipid-altering therapy. Patients currently on statin therapy (with or without ezetimibe) were evaluated by the investigator as to whether this therapy could be safely discontinued at screening, or if it should have been continued. If statin therapy (with or without ezetimibe) was to be continued, dose(s) must have been stable for ≥4 weeks prior to randomization. Patients taking non-statin, lipid-altering medications (niacin >200 mg/day, fibrates, fish oil, other products containing omega-3 fatty acids, or other herbal products or dietary supplements with potential lipid-altering effects), either alone or in combination with statin therapy (with or without ezetimibe), must have been able to safely discontinue non-statin, lipid-altering therapy at screening.

Approximately 240 patients were randomized at approximately 50 centers in North America, South America, Central America, Europe, India, and South Africa. The study was a 58- to 60-week, Phase 3, multi-center study consisting of 3 study periods: (1) a 6- to 8-week screening period that included a diet and lifestyle stabilization and washout period and a TG qualifying period; (2) a 12-week, double-blind, randomized, placebo-controlled treatment period; and (3) a 40-week, open-label, extension period.

During the screening period and double-blind treatment period, all visits were within ±3 days of the scheduled time. During the open-label extension period, all visits were within ±7 days of the scheduled time. The screening period included a 4- or 6-week diet and lifestyle stabilization period and washout period followed by a 2-week TG qualifying period.

The screening visit (Visit 1) occurred for all patients at either 6 weeks (for patients not on lipid-altering therapy at screening or for patients who did not need to discontinue their current lipid-altering therapy) or 8 weeks (for patients who required washout of their current lipid-altering therapy at screening) before randomization, as follows:

Patients who did not require a washout: The screening visit will occur at Visit 1 (Week −6). Eligible patients entered a 4-week diet and lifestyle stabilization period. At the screening visit, all patients received counseling regarding the importance of the National Cholesterol Education Program (NCEP) Therapeutic Lifestyle Changes (TLC) diet and received instructions on how to follow this diet. Patients who required a washout: The screening visit occurred at Visit 1 (Week −8). Eligible patients began a 6-week washout period at the screening visit. Patients received counseling regarding the NCEP TLC diet and received instructions on how to follow this diet. Site personnel contacted patients who did not qualify for participation based on screening laboratory test results to instruct them to resume their prior lipid-altering medications.

At the end of the 4-week diet and lifestyle stabilization period or the 6-week diet and stabilization and washout period, eligible patients entered the 2-week TG qualifying period and had their fasting TG level measured at Visit 2 (Week −2) and Visit 3 (Week −1). Eligible patients must have had an average fasting TG level ≥500 mg/dL and ≤1500 mg/dL (≥5.65 mmol/L and <16.94 mmol/L) to enter the 12-week double-blind treatment period. The TG level for qualification was based on the average (arithmetic mean) of the Visit 2 (Week −2) and Visit 3 (Week −1) values. If a patient's average TG level from Visit 2 and Visit 3 fell outside the required range for entry into the study, an additional sample for fasting TG measurement was collected 1 week later at Visit 3.1. If a third sample was collected at Visit 3.1, entry into the study was based on the average (arithmetic mean) of the values from Visit 3 and Visit 3.1.

After confirmation of qualifying fasting TG values, eligible patients entered a 12-week, randomized, double-blind treatment period. At Visit 4 (Week 0), patients were randomly assigned to one of the following treatment groups:
AMR101 2 g daily,
AMR101 4 g daily, or
Placebo.

During the double-blind treatment period, patients returned to the site at Visit 5 (Week 4), Visit 6 (Week 11), and Visit 7 (Week 12) for efficacy and safety evaluations.

Patients who completed the 12-week double-blind treatment period were eligible to enter a 40-week, open-label, extension period at Visit 7 (Week 12). All patients received open-label AMR101 4 g daily. From Visit 8 (Week 16) until the end of the study, changes to the lipid-altering regimen were permitted (e.g., initiating or raising the dose of statin or adding non-statin, lipid-altering medications to the regimen), as guided by standard practice and prescribing information. After Visit 8 (Week 16), patients returned to the site every 12 weeks until the last visit at Visit 11 (Week 52).

Eligible patients were randomly assigned at Visit 4 (Week 0) to orally receive AMR101 2 g daily, AMR101 4 g daily, or placebo for the 12-week double-blind treatment period. AMR101 was provided in 1 g liquid-filled, oblong, gelatin capsules. The matching placebo capsule was filled with light liquid paraffin and contained 0 g of AMR101. During the double-blind treatment period, patients took 2 capsules (AMR101 or matching placebo) in the morning and 2 in the evening for a total of 4 capsules per day. Patients in the AMR101 2 g/day treatment group received 1 AMR101 1 g capsule and 1 matching placebo capsule in the morning and in the evening. Patients in the AMR101 4 g/day treatment group received 2 AMR101 1 g capsules in the morning and evening.

Patients in the placebo group received 2 matching placebo capsules in the morning and evening. During the extension period, patients received open-label AMR101 4 g daily. Patients took 2 AMR101 1 g capsules in the morning and 2 in the evening.

The primary efficacy variable for the double-blind treatment period was percent change in TG from baseline to Week 12 endpoint. The secondary efficacy variables for the double-blind treatment period included the following:
Percent changes in total cholesterol (TC), high-density lipoprotein cholesterol (HDL-C), calculated low-density lipoprotein cholesterol (LDL-C), calculated non-high-density lipoprotein cholesterol (non-HDL-C), and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A-I/apo B ratio from baseline to Week 12;
Percent changes in lipoprotein(a) from baseline to Week 12 (selected sites only);
Percent changes in LDL particle number and size, measured by nuclear magnetic resonance, from baseline to Week 12 (selected sites only);
Percent change in remnant-like particle cholesterol from baseline to Week 12 (selected sites only);
Percent change in oxidized LDL from baseline to Week 12 (selected sites only);
Changes in FPG and HbA_{1c} from baseline to Week 12;
Change in insulin resistance, as assessed by the homeostasis model index insulin resistance, from baseline to Week 12;
Percent change in lipoprotein associated phospholipase A2 from baseline to Week 12 (selected sites only);
Change in intracellular adhesion molecule-1 from baseline to Week 12 (selected sites only);
Change in interleukin-6 from baseline to Week 12 (selected sites only);
Change in plasminogen activator inhibitor-1 from baseline to Week 12 (selected sites only);
Change in hsCRP from baseline to Week 12 (selected sites only);
Change in serum phospholipid EPA content from baseline to Week 12;
Change in red blood cell membrane EPA content from baseline to Week 12; and
Change in serum phospholipid and red blood cell membrane content in the following fatty acids from baseline to Week 12: docosapentaenoic acid, docosahexaenoic acid, arachidonic acid, palmitic acid, stearic acid, and oleic acid.

The efficacy variable for the open-label extension period was percent change in fasting TG from extension baseline to end of treatment. Safety assessments included adverse events, clinical laboratory measurements (chemistry, hematology, and urinalysis), 12-lead electrocardiograms (ECGs), vital signs, and physical examinations

For TG, TC, HDL-C, calculated LDL-C, calculated non-HDL-C, and VLDL-C, baseline was defined as the average of Visit 4 (Week 0) and the preceding lipid qualifying visit (either Visit 3 [Week −1] or if it occurs, Visit 3.1) measurements. Baseline for all other efficacy parameters was the Visit 4 (Week 0) measurement.

For TC, HDL-C, calculated LDL-C, calculated non-HDL-C, and VLDL-C, Week 12 endpoint was defined as the average of Visit 6 (Week 11) and Visit 7 (Week 12) measurements. Week 12 endpoint for all other efficacy parameters was the Visit 7 (Week 12) measurement.

The primary efficacy analysis was performed using a 2-way analysis of covariance (ANCOVA) model with treatment as a factor and baseline TG value as a covariate. The least-squares mean, standard error, and 2-tailed 95% confidence interval for each treatment group and for each comparison was estimated. The same 2-way ANCOVA model was used for the analysis of secondary efficacy variables.

The primary analysis was repeated for the per-protocol population to confirm the robustness of the results for the intent-to-treat population.

The primary efficacy variable was the percent change in fasting TG levels from baseline to Week 12. A sample size of 69 completed patients per treatment group was expected to provide ≥90% power to detect a difference of 30% between AMR101 and placebo in percent change from baseline in fasting TG levels, assuming a standard deviation of 45% in TG measurements and a significance level of p<0.01. To accommodate a 15% drop-out rate from randomization to completion of the double-blind treatment period, a total of 240 randomized patients was planned (80 patients per treatment group).

### Example 2

A multi-center, placebo-controlled, randomized, double-blind, 12-week study was performed to evaluate the efficacy and safety of >96% E-EPA in patients with fasting triglyceride levels ≥200 mg/dl and <500 mg/dl despite statin therapy (the mean of two qualifying entry values needed to be ≥185 mg/dl and at least one of the values needed to be ≥200 mg/dl). The primary objective of the study was to determine the efficacy of >96% E-EPA 2 g daily and 4 g daily, compared to placebo, in lowering fasting TG levels in patients with high risk for cardiovascular disease and with fasting TG levels ≥200 mg/dl and <500 mg/dl, despite treatment to LDL-C goal on statin therapy.

The secondary objectives of this study were the following:
1. To determine the safety and tolerability of >96% E-EPA 2 g daily and 4 g daily;
2. To determine the effect of >96% E-EPA on lipid and apolipoprotein profiles including total cholesterol (TC), non-high-density lipoprotein cholesterol (non-HDL-C), low density lipoprotein cholesterol (LDL-C), high density lipoprotein cholesterol (HDL-C), and very high density lipoprotein cholesterol (VHDL-C);
3. To determine the effect of >96% E-EPA on lipoprotein associated phospholipase A₂ (Lp-PLA₂) from baseline to week 12;
4. To determine the effect of >96% E-EPA on low-density lipoprotein (LDL) particle number and size;
5. To determine the effect of >96% E-EPA on oxidized LDL;
6. To determine the effect of >96% E-EPA on fasting plasma glucose (FPG) and hemoglobin A_{1c} (HbA_{1c});
7. To determine the effect of >96% E-EPA on insulin resistance;
8. To determine the effect of >96% E-EPA on high-sensitivity C-reactive protein (hsCRP);
9. To determine the effects of >96% E-EPA 2 g daily and 4 g daily on the incorporation of fatty acids into red blood cell membranes and into plasma phospholipids;
10. To explore the relationship between baseline fasting TG levels and the reduction in fasting TG levels; and
11. To explore the relationship between changes of fatty acid concentrations in plasma and red blood cell membranes, and the reduction in fasting TG levels.

The population for this study was men and women >18 years of age with a body mass index ≤45 kg/m² with fasting TG levels greater than or equal to 200 mg/dl and less than 500 mg/dl and on a stable does of statin therapy (with or without ezetimibe). The statin was atorvostatin, rosuvastatin or simvastatin. The dose of statin must have been stable for ≥4 weeks prior to the LDL-C/TG baseline qualifying measurement for randomization. The statin dose was optimized such that the patients are at their LDL-C goal at the LDL-C/TG baseline qualifying measurements. The same statin at the same dose was continued until the study ended.

Patients taking any additional non-statin, lipid-altering medications (niacin >200 mg/day, fibrates, fish oil, other products containing omega-3 fatty acids, or other herbal products or dietary supplements with potential lipid-altering effects), either alone or in combination with statin therapy (with or without ezetimibe), must have been able to safely discontinue non-statin, lipid-altering therapy at screening.

Patients at high risk for CVD, i.e., patients with clinical coronary heart disease (CHD) or clinical CHD risk equivalents (10-year risk >20%) as defined in the National Cholesterol Education Program (NCEP) Adult Treatment Panel III (ATP III) Guidelines were eligible to participate in this study. Those included patients with any of the following criteria: (1) Known CVD, either clinical coronary heart disease (CHD), symptomatic carotid artery disease (CAD), peripheral artery disease (PAD) or abdominal aortic aneurism; or (2) Diabetes Mellitus (Type 1 or 2).

Approximately 702 patients were randomized at approximately 80 centers in the U.S. The study was a 18- to 20-week, Phase 3, multi-center study consisting of 2 study periods: (1) A 6- to 8-week screening period that included a diet and lifestyle stabilization, a non-statin lipid-altering treatment washout, and an LDL-C and TG qualifying period and (2) A 12-week, double-blind, randomized, placebo-controlled treatment period.

During the screening period and double-blind treatment period, all visits were within ±3 days of the scheduled time. All patients continued to take the statin product (with or without ezetimibe) at the same dose they were taking at screening throughout their participation in the study.

The 6- to 8-week screening period included a diet and lifestyle stabilization, a non-statin lipid-altering treatment washout, and an LDL-C and TG qualifying period. The screening visit (Visit 1) occurred for all patients at either 6 weeks (for patients on stable statin therapy [with or without ezetimibe] at screening) or 8 weeks (for patients who will require washout of their current non-statin lipid-altering therapy at screening) before randomization, as follows:
Patients who did not require a washout: The screening visit occurred at Visit 1 (Week −6). Eligible patients entered a 4-week diet and lifestyle stabilization period. At the screening visit, all patients received counseling regarding the importance of the National Cholesterol Education Program (NCEP) Therapeutic Lifestyle Changes (TLC) diet and received basic instructions on how to follow this diet.
Patients who required a washout: The screening visit occurred at Visit 1 (Week −8). Eligible patients began a 6-week washout period at the screening visit (i.e. 6 weeks washout before the first LDL-C/TG qualifying visit). Patients received counseling regarding the NCEP TLC diet and received basic instructions on how to follow this diet. Site personnel contacted patients who did not qualify for participation based on screening laboratory test results to instruct them to resume their prior lipid-altering medications.

At the end of the 4-week diet and lifestyle stabilization period or the 6-week diet and stabilization and washout period, eligible patients entered the 2-week LDL-C and TG qualifying period and had their fasting LDL-C and TG levels measured at Visit 2 (Week −2) and Visit 3 (Week −1). Eligible patients must have had an average fasting LDL-C level ≥40 mg/dL and <100 mg/dL and an average fasting TG level ≥200 mg/dL and <500 mg/dL to enter the 12-week double-blind treatment period. The LDL-C and TG levels for qualification were based on the average (arithmetic mean) of the Visit 2 (Week −2) and Visit 3 (Week −1) values. If a patient's average LDL-C and/or TG levels from Visit 2 and Visit 3 fell outside the required range for entry into the study, an additional fasting lipid profile was collected 1 week later at Visit 3.1. If a third sample was collected at Visit 3.1, entry into the study was based on the average (arithmetic mean) of the values from Visit 3 and Visit 3.1.

After confirmation of qualifying fasting LDL-C and TG values, eligible patients entered a 12-week, randomized, double-blind treatment period. At Visit 4 (Week 0), patients were randomly assigned to 1 of the following treatment groups:
>96% E-EPA 2 g daily,
>96% E-EPA 4 g daily, or
Placebo.

226 to 234 patients per treatment group were randomized in this study. Stratification was by type of statin (atorvastatin, rosuvastatin or simvastatin), the presence of diabetes, and gender.

During the double-blind treatment period, patients returned to the site at Visit 5 (Week 4), Visit 6 (Week 11), and Visit 7 (Week 12) for efficacy and safety evaluations.

Eligible patients were randomly assigned at Visit 4 (Week 0) to receive orally >96% E-EPA 2 g daily, >96% E-EPA 4 g daily, or placebo.

>96% E-EPA was provided in 1 g liquid-filled, oblong, gelatin capsules. The matching placebo capsule was filled with light liquid paraffin and contained 0 g of >96% E-EPA. >96% E-EPA capsules were to be taken with food (i.e. with or at the end of a meal).

During the double-blind treatment period, patients were to take 2 capsules (>96% E-EPA or matching placebo) in the morning and 2 capsules in the evening for a total of 4 capsules per day.
Patients in the >96% E-EPA 2 g/day treatment group received 1 >96% E-EPA 1 g capsule and 1 matching placebo capsule in the morning and in the evening.
Patients in the >96% E-EPA 4 g/day treatment group received 2 >96% E-EPA 1 g capsules in the morning and evening.

Patients in the placebo group received 2 matching placebo capsules in the morning and evening.

The primary efficacy variable for the double-blind treatment period was percent change in TG from baseline to Week 12 endpoint. The secondary efficacy variables for the double-blind treatment period included the following:
Percent changes in total cholesterol (TC), high-density lipoprotein cholesterol (HDL-C), LDL-C, calculated non-HDL-C, and very low-density lipoprotein cholesterol (VLDL-C) from baseline to Week 12 endpoint;
Percent change in very low-density lipoprotein TG from baseline to Week 12;
Percent changes in apolipoprotein A-I (apo A-I), apolipoprotein B (apo B), and apo A-I/apo B ratio from baseline to Week 12;
Percent changes in lipoprotein(a) from baseline to Week 12;
Percent changes in LDL particle number and size, measured by nuclear magnetic resonance, from baseline to Week 12;
Percent change in remnant-like particle cholesterol from baseline to Week 12;
Percent change in oxidized LDL from baseline to Week 12;
Changes in FPG and HbA_{1c} from baseline to Week 12;
Change in insulin resistance, as assessed by the homeostasis model index insulin resistance, from baseline to Week 12;
Percent change in lipoprotein associated phospholipase A₂ (Lp-PLA₂) from baseline to Week 12;
Change in intracellular adhesion molecule-1 from baseline to Week 12;
Change in interleukin-2 from baseline to Week 12;
Change in plasminogen activator inhibitor-1 from baseline to Week 12. Note: this parameter will only be collected at sites with proper storage conditions;
Change in hsCRP from baseline to Week 12; and
Change in plasma concentration and red blood cell membrane content of fatty acid from baseline to Week 12 including EPA, docosapentaenoic acid (DPA), docosahexaenoic acid (DHA), arachidonic acid (AA), dihomo-γ-linolenic acid (DGLA), the ratio of EPA/AA, ratio of oleic acid/stearic acid (OA/SA), and the ratio of total omega-3 acids over total omega-6 acids.

Safety assessments included adverse events, clinical laboratory measurements (chemistry, hematology, and urinalysis), 12-lead electrocardiograms (ECGs), vital signs, and physical examinations.

For TG, TC, HDL-C, LDL-C, calculated non-HDL-C, and VLDL-C, baseline was defined as the average of Visit 4 (Week 0) and the preceding lipid qualifying visit (either Visit 3 [Week −1] or if it occurs, Visit 3.1) measurements. Baseline for all other efficacy parameters was the Visit 4 (Week 0) measurement.

For TG, TC, HDL-C, LDL-C, calculated non-HDL-C, and VLDL-C, Week 12 endpoint was defined as the average of Visit 6 (Week 11) and Visit 7 (Week 12) measurements.

Week 12 endpoint for all other efficacy parameters were the Visit 7 (Week 12) measurement.

The primary efficacy analysis was performed using a 2-way analysis of covariance (ANCOVA) model with treatment as a factor and baseline TG value as a covariate. The least-squares mean, standard error, and 2-tailed 95% confidence interval for each treatment group and for each comparison were estimated. The same 2-way ANCOVA model was used for the analysis of secondary efficacy variables.

The primary analysis was repeated for the per-protocol population to confirm the robustness of the results for the intent-to-treat population.

Non-inferiority tests for percent change from baseline in LDL-C were performed between >96% E-EPA doses and placebo using a non-inferiority margin of 6% and a significant level at 0.05.

For the following key secondary efficacy parameters, treatment groups were compared using Dunnett's test to control the Type 1 error rate: TC, LDL-C, HDL-C, non-HDL-C, VLDL-C, Lp-PLA₂, and apo B. For the remaining secondary efficacy parameters, Dunnett's test was be used and the ANCOVA output were considered descriptive.

The evaluation of safety was based primarily on the frequency of adverse events, clinical laboratory assessments, vital signs, and 12-lead ECGs. The primary efficacy variable is the percent change in fasting TG levels from baseline to Week 12. A sample size of 194 completed patients per treatment group provided 90.6% power to detect a difference of 15% between >96% E-EPA and placebo in percent change from baseline in fasting TG levels, assuming a standard deviation of 45% in TG measurements and a significance level of p<0.05.

Previous data on fasting LDL-C show a difference in percent change from baseline of 2.2%, with a standard deviation of 15%, between study drug and placebo. A sample size of 194 completed patients per treatment group provided 80% power to demonstrate non-inferiority (p<0.05, one-sided) of the LDL-C response between >96% E-EPA 4 g daily and placebo, within a 6% margin. To accommodate a 10% drop-out rate from randomization to completion of the double-blind treatment period, a total of 648 randomized patients was planned (216 patients per treatment group); 702 subjects were randomized, as further described below.

### Results

Of the 702 randomized subjects, 687 were in the intent-to-treat (“ITT”) population as follows:
Ultra-pure EPA, 4 g/day: 226 subjects
Ultra-pure EPA, 2 g/day: 234 subjects
Placebo: 227 subjects

Lipids were extracted from plasma and red blood cell (“RBC”) suspensions and converted into fatty acid methyl esters for analysis using a standard validated gas chromatography/flame ionization detection method. Fatty acid parameters were compared between EPA treatment groups and placebo using an ANCOVA model with treatment, gender, type of statin therapy, and presence of diabetes as factors, and the baseline parameter value as a covariate. LSMs, SEs, and 2-tailed 95% confidence intervals for each treatment group and for each comparison were determined.

Baseline characteristics of the three ITT groups were comparable, with 61.4% of the ITT subjects being male, 96.3% being white, having a mean age of 61.4 years, a weight of 95.7 kg and a BMI of 32.9 kg/m². ITT subjects with incomplete fatty acid data at baseline and/or at 12 weeks were excluded from the analyses described below.

### Example 3

An experiment was conducted to test EPA in model membranes enriched with PUFAs and cholesterol at levels that reproduce disease or high CV-risk conditions (i.e., hypercholesterolemia).

The effects of EPA on lipid peroxide (LOOH) formation were examined at a cholesterol-to-phospholipid (C/P) mole ratio of 0.6:1. Levels of lipid hydroperoxides were also measured in cholesterol-enriched membrane prepared in the absence of EPA as a control.

1,2-Dilinoleoyl-3-sn-phosphatidylcholine (DLPC) was obtained from Avanti Polar Lipids (Alabaster, Ala.) and stored in chloroform (25 mg/ml) at −80° C. until use. Cholesterol obtained and stored in chloroform (10 mg/ml) at −20° C. CHOD-iodide color reagent (stock) was prepared according to a procedure modified from El-Saadani et al. (El-Saadani M, Esterbauer H, El-Sayed M, Goher M, Nassar A Y, Jurgens G. A spectrophotometric assay for lipid peroxides in serum lipoproteins using commercially available reagent. J. Lipid. Res. 1989; 30:627-30) consisted of 0.2 M K₂HPO₄, 0.12 M KI, 0.15 mM NaN₃, 10 μM ammonium molybdate, and 0.1 g/L benzalkonium chloride. Prior to experimental use, the CHOD reagent was activated by adding 24 μM ethylenediaminetetraacetic acid (EDTA), 20 μM butylated hydroxytoluene (BHT), and 0.2% Triton X-100. The EPA and lipids were added in a ratio of 1:30 during membrane sample preparation to ensure full incorporation into the lipid bilayers.

Membrane samples consisting of DLPC±cholesterol were prepared as follows. Component lipids (in chloroform) were transferred to 13×100 mm test tubes and shell-dried under a steady stream of nitrogen gas while vortex mixing. The lipid was co-dried with EPA.

Residual solvent was removed by drying for a minimum of 3 h under vacuum. After desiccation, each membrane sample was resuspended in diffraction buffer (0.5 mM HEPES, 154 mM NaCl, pH 7.3) to yield a final phospholipid concentration of 1.0 mg/mL. Multilamellar vesicles (MLV) were formed by vortex mixing for 3 minutes at ambient temperature. Bangham A D, Standish M M, Watkins J C. Diffusion of univalent ions across the lamellae of swollen phospholipids. J. Mol. Biol. 1965; 13:238-52. Immediately after initial MLV preparation, aliquots of each membrane sample will be taken for baseline (0 h) peroxidation analyses.

All lipid membrane samples were subjected to time-dependent autoxidation by incubating at 37° C. in an uncovered, shaking water bath for 72 hours. Small aliquots of each sample were removed at 24 h intervals and combined with 1.0 mL of active CHOD-iodide color reagent. To ensure spectrophotometric readings within the optimum absorbance range, sample volumes taken for measurement of lipid peroxide formation were adjusted for length of peroxidation and range between 100 and 10 μL. Test samples were immediately covered with foil and incubated at room temperature for >4 h in the absence of light. Absorbances were measured against a CHOD blank at 365 nm using a Beckman DU-640 spectrophotometer.

The CHOD colorimetric assay is based on the oxidation of iodide (I⁻) by lipid hydroperoxides (LOOH) and proceeds according to the following reaction scheme:

LOOH+2H⁺+3I⁻→LOH+H₂O+I₃⁻

The quantity of triiodide anion (I₃⁻) liberated in this reaction is directly proportional to the amount of lipid hydroperoxides present in the membrane sample. The molar absorptivity value (ε) of I₃⁻ is 2.46×10⁴ M⁻¹·cm⁻¹ at 365 nm.

Cholesterol domain peak intensity was calculated from multiple small angle x-ray diffraction measurements, which are directly proportional to domain levels. After exposure to autoxidation as described above, vehicle-treated controls displayed a cholesterol domain peak intensity of 77.6±58.5, corresponding to an increase in LOOH formation from 89±1 μM to 6616±250 μM (p<0.001). EPA-treated membranes reduced LOOH levels by greater than 90% (728±30 μM) compared to untreated controls (p<0.001).

This example demonstrates that EPA inhibits cholesterol crystalline domain formation in a manner related to its potent antioxidant effects in PUFA-enriched model membranes. These data suggest that EPA blocks membrane lipid oxidation and structural reorganization through free radical chain-breaking mechanisms.

### Example 4

An experiment was conducted to test the ability of EPA to interfere with the effects of high glucose on membrane lipid peroxidation and organization in vesicles enriched with PUFAs.

At elevated levels, the aldose sugar glucose produces non-enzymatic chemical modifications to membrane proteins and phospholipids, leading to advanced glycation endproducts (AGEs) and cell injury. Oxidative stress and AGEs have been implicated in both the microvascular and macrovascular complications of diabetes and other metabolic disorders. In membranes enriched with polyunsaturated fatty acids (PUFA), hyperglycemia promotes the formation of free radicals and cholesterol crystalline domains associated with atherosclerosis. The non-enzymatic effects of glucose on cholesterol crystalline domain formation were shown to be enhanced under conditions of high cholesterol and could not be reproduced by mannitol. Oxidative damage to PUFAs with glucose is of particular interest given its role in the propagation of free radicals during vascular injury and insulin resistance. In addition to cellular membranes, oxidation of PUFAs in low-density lipoproteins (LDL) contributes to endothelial dysfunction, inflammation, and atherosclerotic foam cell formation.

1,2-Dilinoleoyl-sn-glycero-3-phosphocholine (DLPC) and monomeric cholesterol (isolated from ovine wool) were purchased from Avanti Polar Lipids (Alabaster, Ala.) and solubilized at 25 and 10 mg/mL, respectively. EPA, α-linolenic acid (ALA; 18:3, n−3) was purchased from Sigma-Aldrich (Saint Louis, Mo.) and solubilized in ethanol to 1 mM under nitrogen atmosphere. Vitamin E (α-tocopherol) was also purchased from Sigma-Aldrich and prepared in ethanol at 1.0 mM (ε=3.06×10⁴ M⁻¹ cm⁻¹ at 294 nm) just prior to experimental use. Atorvastatin ortho- (o-) hydroxy (active) metabolite was purchased from Toronto Research Chemicals (North York, Ontario, Canada) and solubilized in methanol to 1.0 mM. All test compounds were further diluted in ethanol or aqueous buffer as needed. Glucose was prepared in saline buffer (0.5 mM HEPES, 154 mM NaCl, pH 7.3) at 11.0 mM (200 mg/dL).

CHOD-iodide color reagent (stock) was prepared, with slight modification, as described by from El-Saadani et al. (J. Lipid Res., vol. 30, pages 627-630 (1989)) and consisted of 0.2 M K₂HPO₄, 0.12 M KI, 0.15 mM NaN₃, 10 μM ammonium molybdate, and 0.1 g/L benzalkonium chloride. Prior to experimental use, the CHOD reagent was activated by adding 24 μM ethylenediaminetetraacetic acid (EDTA), 20 μM butylated hydroxytoluene (BHT), and 0.2% Triton X-100.

Multilamellar vesicles (MLVs) were prepared as binary mixtures of DLPC (1.0 or 2.5 mg total phospholipid per sample) and cholesterol at a fixed cholesterol-to-phospholipid (C/P) mole ratio of 0.6:1. Component lipids (in chloroform) were transferred to 13×100 mm borosilicate culture tubes and combined with vehicle (ethanol) or an equal volume of fatty acid, vitamin E, or ATM stock solutions, each adjusted to achieve desired treatment concentrations. Samples were shell-dried under nitrogen gas and placed under vacuum for 1 h to remove residual solvent. After desiccation, each sample was resuspended in 1.0 mL glucose-containing saline to yield final phospholipid concentrations of 1.0 or 2.5 mg/mL (for lipid peroxidation or x-ray diffraction analysis, respectively). Lipid suspensions were then vortexed for 3 min at ambient temperature to form MLVs.

All MLV samples were subjected to time-dependent autoxidation by incubating at 37° C. in an uncovered, shaking water bath. This method allows lipid peroxidation to occur gradually without requiring the use of exogenous initiators. Small aliquots (5-100 μL) of each sample were removed, immediately following MLV preparation (0 hour) and after exposing samples to oxidative conditions for 72 or 96 hour, and combined with 1.0 mL of activated CHOD-iodide color reagent. Aliquot volume was reduced with each successive time point to ensure that spectrophotometric readings were within the optimal adsorption range. Test samples were covered and incubated in darkness at room temperature for at least 4 hr. Sample absorbances were then measured against a CHOD blank at 365 nm using a Beckman DU-640 spectrophotometer. The CHOD colorimetric assay is based on the oxidation of iodide (I⁻) by lipid hydroperoxide (LOOH) to form triiodide (I₃⁻), the quantity of which is directly proportional to the amount of LOOH present in the lipid sample. The molar absorptivity (E) of I₃⁻ is 2.46×10⁴ M⁻¹ cm⁻¹ at 365 nm.

The membrane structural effects of glucose and the various compounds examined in this study were measured at 0, 72, and 96 hour intervals. Membrane lipid vesicles were oriented for x-ray diffraction analysis as described by others (e.g., Herbette et al., Biophys. J., vol. 20(2), pages 245-272 (1977)). Briefly, a 100 μL aliquot (containing 250 μg MLV) was aspirated from each sample and transferred to a Lucite® sedimentation cell fitted with an aluminum foil substrate upon which a given sample could be collected by centrifugation. Samples were then loaded into a Sorvall AH-629 swinging bucket rotor (DuPont Corp., Wilmington, Del.) and centrifuged at 35,000 g, 5° C., for 90 min.

After centrifugal orientation, sample supernatants were aspirated and aluminum foil substrates, each supporting a single membrane pellet, were removed from the sedimentation cells. Sample pellets were dried for 5-10 min at ambient conditions, mounted onto curved glass supports, and placed in hermetically-sealed, brass or glass containers (for immediate analysis or temporary storage, respectively). All x-ray diffraction experiments were conducted at 20° C., 74% relative humidity. The latter was established by exposing membrane samples to saturated solutions of L-(+) tartaric acid (K₂C₄H₄O₆.½H₂O). Samples were incubated at these conditions for at least 1 hour prior to experimental analysis.

Oriented membrane samples were aligned at grazing incidence with respect to a collimated, mono-chromatic CuK_{α} x-ray beam (K_{α1} and K_{α2} unresolved; λ=1.54 Å) produced by a Rigaku Rotaflex RU-200, high-brilliance microfocus generator (Rigaku-MSC, The Woodlands, Tex.) as previously described (Mason et al., Biophys. J., vol. 55(4), pages 769-778 (1989)). Diffraction data were collected on a one-dimensional, position-sensitive electron detector (Hecus X-ray Systems, Graz, Austria) at a sample-to-detector distance of 150 mm. Detector calibration was performed by the manufacturer and verified using crystalline cholesterol monohydrate.

The d-space for any given membrane multibilayer is a measurement of the unit cell periodicity of the membrane lipid bilayer (e.g., the distance from the center of one lipid bilayer to the next including surface hydration), and is calculated from Bragg's Law, hλ=2d sin θ, where h is the diffraction order, λ is the wavelength of the x-ray radiation (1.54 Å), d is the membrane lipid bilayer unit cell periodicity, and θ is the Bragg angle equal to one-half the angle between the incident beam and scattered beam.

The presence of cholesterol domains in a given membrane sample results in the production of distinct Bragg (diffraction) peaks having singular periodicity values of 34 and 17 Å (typically referred to as first- and second-order cholesterol domain peaks). Under the specific temperature and relative humidity conditions established for these experiments, the second-order, 17 Å cholesterol domain peak was well-delineated from other, neighboring cholesterol and phospholipid diffraction peaks and was thus used to quantitate relative cholesterol domain peak intensity. Routines written in Origin 8.6 (OriginLab Corporation, Northampton, Mass.) were used to determine total peak area (associated with all diffraction peaks in a given pattern) against which the second-order cholesterol domain peak was normalized.

Effects of EPA and Vitamin E on Glucose-induced Lipid Peroxidation

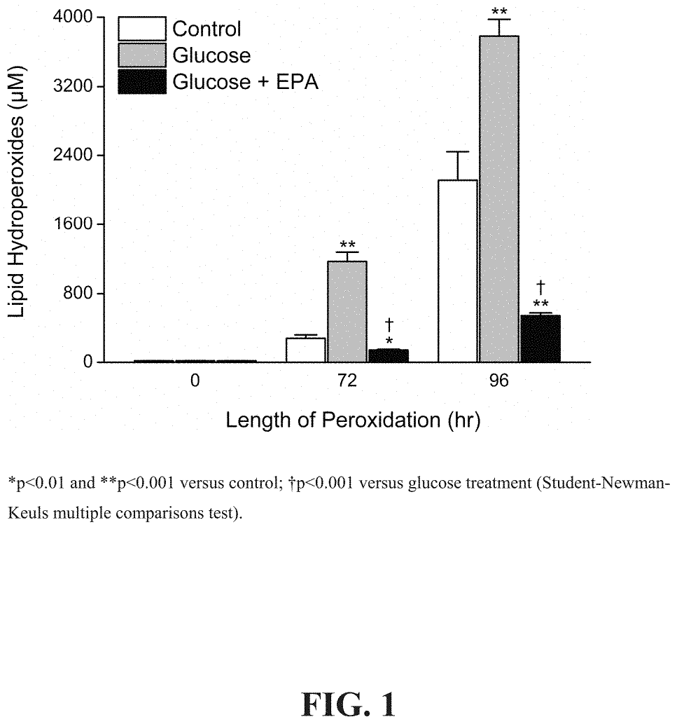

The effects of hyperglycemia (200 mg/dL) on LOOH formation in lipid vesicles enriched with PUFAs and cholesterol and prepared in the absence (vehicle only) or presence of EPA or vitamin E (each at a 1:30 drug-to-phospholipid mole ratio) were measured. The concentration of glucose selected was consistent with previous experimental studies of hyperglycemia under controlled laboratory conditions or observed in well-defined animal models of Type II diabetes following a glucose challenge. As shown in FIG. 1, glucose significantly increased LOOH formation in a time-dependent manner as compared to vehicle treatment alone. Values in FIG. 1 are mean±S.D. (N=6). EPA inhibited the peroxidative effects of glucose by 88% and 86% at 72 and 96 hours, respectively, which was highly significant (p<0.001) as compared to glucose treatment alone. LOOH levels measured in samples treated with EPA were also significantly lower (at the 72- and 96-hour time points) as compared to non-glucose-treated controls. Overall ANOVA-0 hour data: p=0.6655, F=0.4185; 72 hour data: p<0.0001, F=428.72; 96 hour data: p<0.0001, F=322.01.

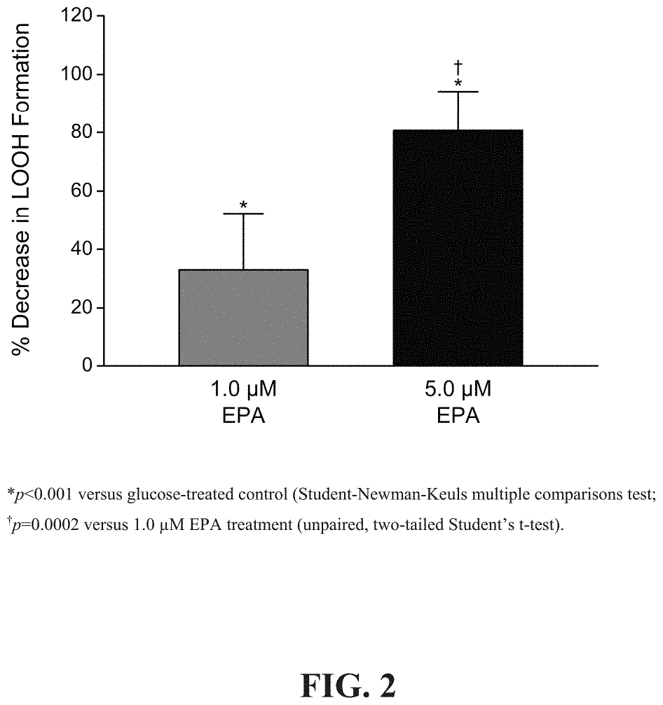

EPA was also tested at 1.0 and 5.0 μM and found to inhibit membrane LOOH formation in a dose-dependent manner, as shown in FIG. 2. Values in FIG. 2 are mean±S.D. (N=6-8) and represent % difference between treatment and glucose-treated controls. These concentrations are similar to those measured in the plasma of patients with prescribed levels of EPA. Overall ANOVA: p<0.0001, F=99.900.

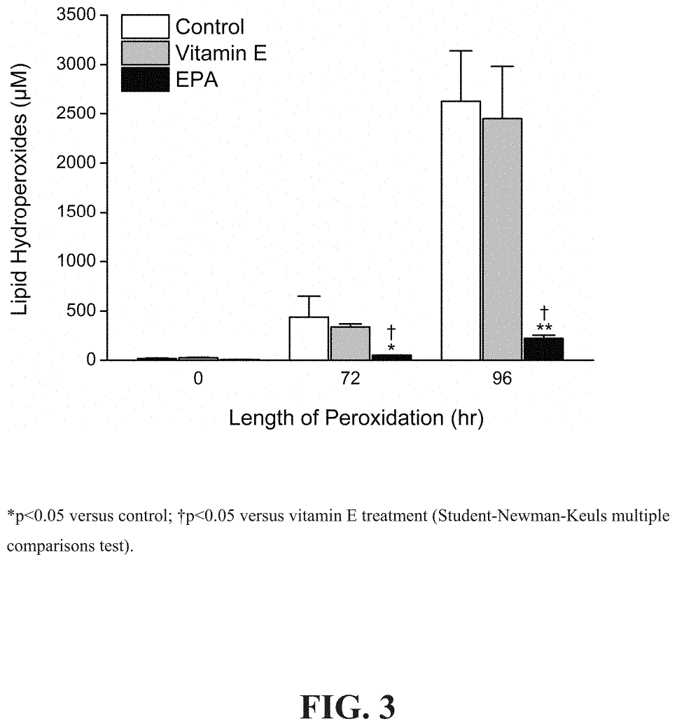

Vitamin E was also examined in this assay at the same drug-to-phospholipid mole ratio used for testing the basic antioxidant effects of EPA. As shown in FIG. 3, EPA significantly inhibited LOOH formation at the 72- and 96-hour time points. Values in FIG. 3 are mean±S.D. (N=3). By contrast, vitamin E had no significant effect on lipid peroxidation under identical conditions. Overall ANOVA-0 hour data: p=0.0073, F=12.474; 72 hour data: p=0.0204, F=7.986; 96 hour data: p=0.0008, F=29.764.

### Effects of EPA and Vitamin E on Glucose- and Peroxidation-Induced Changes in Membrane Lipid Structural Organization

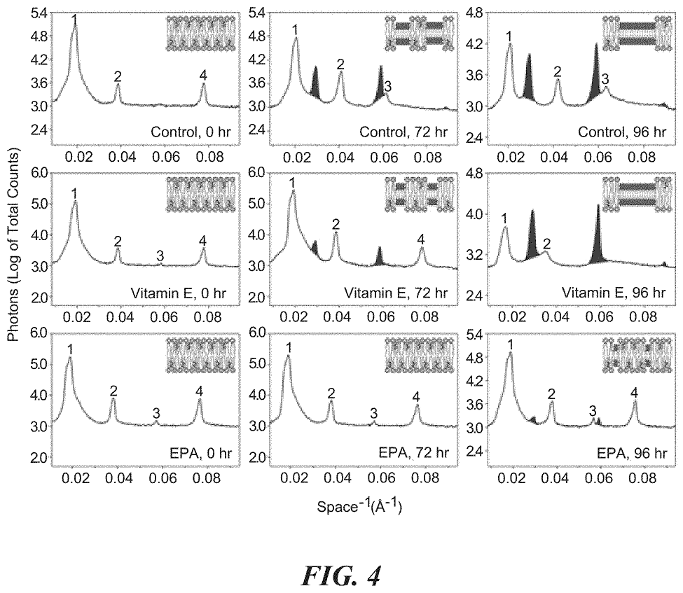

Lipid peroxidation is highly disruptive to the structural organization of biological membranes and has been shown, in previous studies by Jacob et al. Biol. Chem., vol. 280, pages 39380-39387 (2005)) and Mason et al. (J. Biol. Chem., vol. 281(1), pages 9337-9345 (2006)), to contribute directly to the formation of cholesterol crystalline domains. Glucose has also been reported to promote similar changes in membrane structural organization by increasing lipid peroxidation (Self-Medlin et al., Biochim. Biophys. Acta, vol. 1788(6), pages 1398-1403 (2009)). In this study, we used small angle x-ray diffraction to characterize the structural properties of model membranes treated with glucose (200 mg/dL) and prepared in the absence or presence of vitamin E or EPA (each at 1:30 drug-to-phospholipid mole ratio), before and after exposure to oxidative conditions (FIG. 4). At the start of this experiment, vitamin E and EPA were observed to have no appreciable effect on membrane structure as compared to control samples (FIG. 4, left column). Scattering data collected from each membrane preparation yielded up to four diffraction orders having an average unit cell periodicity (d-space) of 51.5 Å, and consistent with a homogenously-distributed, lipid bilayer phase. Following exposure to oxidative conditions for 72 hours, additional peaks, with an average d-space value of 34 Å and consistent with a cholesterol crystalline domain phase, were observed in control and vitamin E-treated membrane samples (FIG. 4, middle column, highlighted peaks). At 96 hours, cholesterol domains peaks were observed in all experimental samples; however, these peaks were disproportionately greater in control and vitamin E-treated samples (FIG. 4, right column, highlighted peaks).

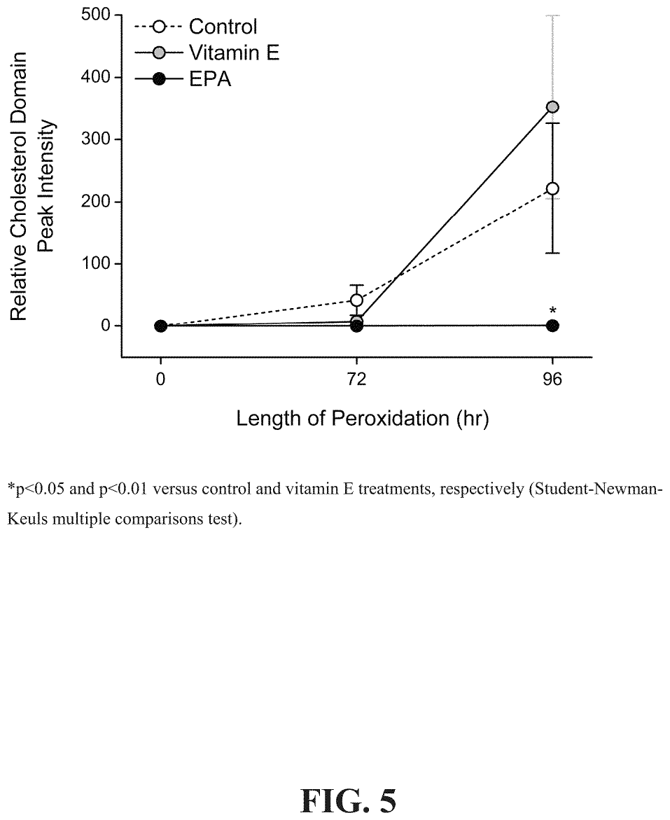

Quantitative assessment of cholesterol domain peak intensity (expressed as the quotient of cholesterol- to total lipid-peak area) indicated that vitamin E had no significant effect on cholesterol domain formation as compared to control at any experimental time point (FIG. 5). Values in FIG. 5 are mean±S.D. (N=3). In contrast, EPA inhibited relative cholesterol domain peak intensity by more than 99% at the 96-hour time point, as compared to either vehicle or vitamin E treatments. Overall ANOVA: p=0.0075, F=8.849.

### Separate and Combined Effects of EPA and ATM on Glucose-Induced Membrane Lipid Peroxidation

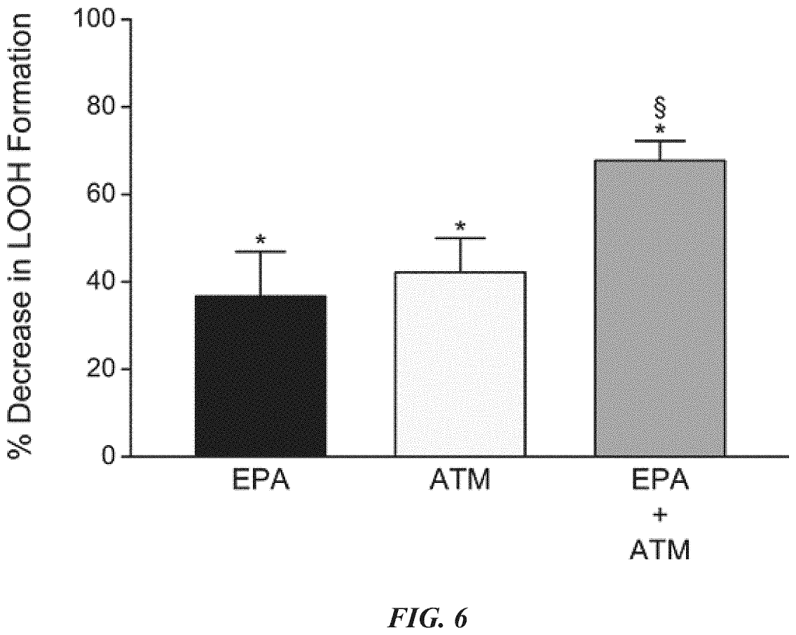

ATM has been shown in previous studies by Teissier et al. (Circ. Res., vol. 95(12), pages 1174-1182 (2004)) and Mason et al. (Am. J. Cardiol., vol. 96(5A), pages 11F-23F (2005)) to have potent antioxidant properties, as observed in human low density lipoprotein as well as model liposomes. The antioxidant effects of ATM were re-examined, separately and in combination with EPA (each at 1.0 μM), in membrane lipid vesicles treated with glucose at 200 mg/dL and exposed to oxidative conditions for 96 hours. Both EPA and ATM were observed to have separate and potent antioxidant effects under these conditions; however, their combination was even more effective, decreasing LOOH formation by >60% (p<0.001) as compared to either treatment alone (FIG. 6). Values in FIG. 6 are mean±S.D. (N=6) and represent % difference between treatment and glucose-treated controls. *p<0.001 versus glucose-treated control (Student-Newman-Keuls multiple comparisons test; overall ANOVA: p<0.0001, F=111.69. ^{§} p<0.001 versus separate EPA or ATM treatments (Student-Newman-Keuls multiple comparisons test; overall ANOVA: p<0.0001, F=26.635).

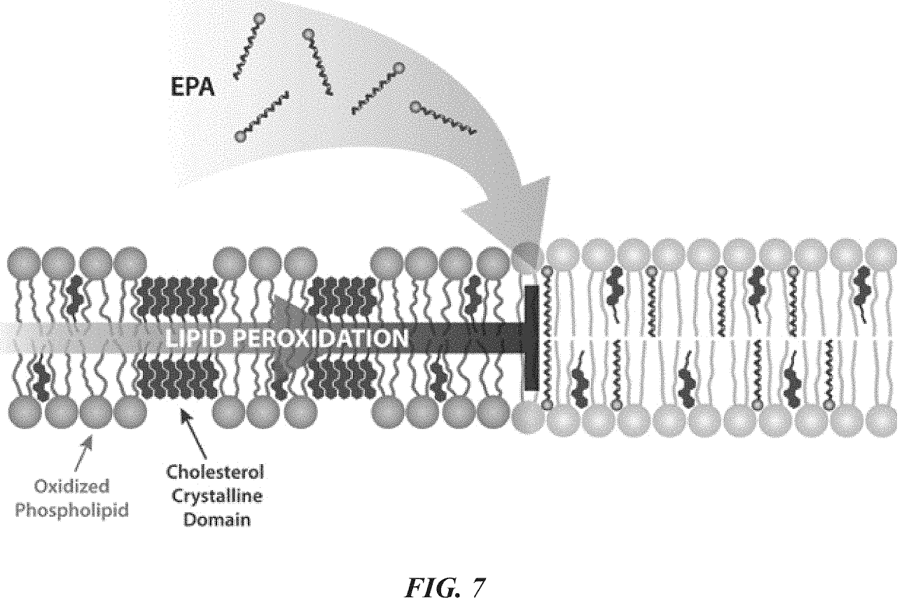

These data demonstrate that EPA significantly inhibits glucose-induced lipid peroxidation and cholesterol crystalline domain formation in model membrane lipid vesicles. Without wishing to be bound by theory, it is possible that these antioxidant effects are due at least in part to the ability of EPA to quench reactive oxygen species (ROS) associated with the membrane lipid bilayer, thereby preserving normal membrane structure and organization. Following intercalation into the membrane lipid bilayer, the conjugated double bonds associated with EPA may facilitate electron stabilization mechanisms that interfere with free radical propagation (for example, as depicted in FIG. 7). The effects of EPA could not be reproduced with vitamin E, a natural scavenging antioxidant. These findings indicate a potentially preferred intercalation of the EPA molecule into the membrane where it can trap free radicals. The absence of activity for vitamin E under these conditions may be due to its limited lipophilicity and scavenging potential, as previously observed by Mason et at (J. Biol. Chem., vol. 281(14), pages 9337-9345 (2006)) in membranes enriched with cholesterol. Vitamin E was also unable to interfere with cholesterol crystalline domain development with hyperglycemia. Finally, the antioxidant effects of EPA were enhanced in the presence of ATM which, unlike vitamin E, has been shown by Self-Medlin et al. (Biochim. Biophys. Acta, vol. 1788(6), pages 1398-1403 (2009)) and Mason et a. (J. Biol. Chem., vol. 281(14), pages 9337-9345 (2006)) to have potent free radical scavenging properties and high lipophilicity that reduces the formation of cholesterol crystalline domains following oxidative stress or exposure to hyperglycemic conditions. Mason et at (J. Biol. Chem., vol. 281(14), pages 9337-9345 (2006)) and Aviram et a. (Atherosclerosis, vol. 138(2), pages 271-280 (1998)) have attributed the chain-breaking antioxidant mechanism of ATM to its phenoxy moiety. Clinical support for an antioxidant benefit with atorvastatin has also been reported from prospective trials by Tsimikas et a. (Circulation, vol. 110(11), pages 1406-1412 (2004)) and Shishehbor et al. (Circulation, vol. 108(4), pages 426-431 (2003)).

At high levels, glucose promoted the formation of LOOH, prominent intermediates of peroxidative reactions that have been shown by Girotti et a. (J. Lipid Res., vol. 39(8), pages 1529-1542 (1998)) to lead to changes in the organization of membrane lipid components. Lipid peroxidation is well-known to induce changes in membrane fluidity, increased membrane permeability, and changes in membrane protein activity. Oxidative modification of PUFAs is also known to cause a marked reduction in membrane d-space associated with interdigitation of the phospholipid acyl chain terminal methyl segments. These alterations in the intermolecular packing characteristics of membrane phospholipids promote the displacement of cholesterol into discrete domains (d-space of 34 Å) within the phospholipid bilayer environment. Cholesterol crystalline domains have been shown by Ruocco et al. (Biophys. J., vol. 46, pages 695-707 (1984)) to be induced in model membranes by increasing membrane cholesterol to very high levels (>50 mol %). Similar changes in cholesterol domain formation have been observed in models of atherosclerosis, including in model macrophage foam cells (Kellner-Weibel et al., Arterioscler. Thromb. Vasc. Biol., vol. 19(8), pages 1891-1898 (1999)), in rabbit or rat thoracic mesenteric or pericardium membranes (Abela et al., Clin. Cardiol., vol. 28(9), pages 413-420 (2005)), and rabbit smooth muscle cell plasma membranes (Tulenko et al., J. Lipid Res., vol. 39, pages 947-956 (1998)). The formation of these domains in membranes prepared at constant cholesterol levels but exposed to glucose and glucose-induced lipid peroxidation has also been observed by Self-Medlin et al. (Biochim. Biophys. Acta, vol. 1788(6), pages 1398-1403 (2009)). Thus, agents that slow or block the formation of cholesterol into discrete domains and crystals may interfere with mechanisms of atherogenesis associated with hyperglycemia without reductions in cholesterol levels.

As a reducing monosaccharide, glucose is known to be susceptible to reaction at its anomeric carbon with singlet oxygen or other radical initiators. This redox reaction can generate glucose radicals or other reactive oxygen species that have a pro-oxidant effect in biological membranes. Several reaction mechanisms are believed to be responsible for the formation of glycoxidation and lipoxidation products resulting from the reaction of glucose radicals with proteins or lipids to form sugar-amine adducts. The presence of cholesterol in the membrane also contributes to rates of LOOH formation, allowing more efficient radical penetration and propagation through the bilayer. The steroid nucleus of cholesterol has been explained to induce an ordering effect on adjacent phospholipid molecules, thus reducing the intermolecular distance between adjacent PUFA chains of the lipids and facilitating the exchange of free radicals within the hydrocarbon core. Previous studies by Self-Medlin et al. (Biochim. Biophys. Acta, vol. 1788(6), pages 1398-1403 (2009)) have demonstrated a cholesterol-dependent increase in LOOH formation, which was enhanced by glucose treatment, in similar model membrane preparations. Others including Bertelsen et al. (Diabetologia, vol 44(5), pages 605-613 (2001)) and Cohen et al. (Am. J. Physiol. Endocrin. Metabol., vol. 285(6), pages E1151-1160 (2003)) have suggested that even minor physico-chemical modifications to the cell membrane may lead to the disruption of cholesterol-enriched membrane domains that are critical to many cellular processes (e.g., caveolae) leading to loss in insulin receptor activity and endothelial nitric oxide synthase (eNOS) function.

Glucose-mediated oxidative stress is known to contribute to inflammatory pathways associated with diabetes and atherosclerosis pathophysiology. Glucose, obesity, and oxidative stress reduce intracellular antioxidant defense mechanisms while activating inflammatory responses from transcription factors and kinases, such as c-Jun N-terminal kinase (JNK), protein kinase C (PKC), and inhibitor of kappa B kinase-β (IKKβ). Some inflammatory pathways, such as activation of IKKβ, have a causative role in the deleterious effects of hyperglycemia on endothelial cell function. Hyperglycemia also stimulates NF-kB, which in turn promotes the overexpression of NADPH, a primary source of cellular superoxide. Overproduction of superoxide, accompanied by increased nitric oxide generation, leads to formation of the highly reactive peroxynitrite molecule. Agents with antioxidant activity at the cellular level including, for example, statins, glitazones, and angiotensin converting enzyme (ACE) inhibitors, have been shown to be beneficial in improving insulin resistance.

The present Example demonstrates surprisingly that EPA ameliorates the effects of hyperglycemia, likely due to its potent antioxidant properties. In clinical studies performed by others, compositions including EPA reduced CAD-related events in hypercholesterolemic patients receiving statin treatment. In addition to reductions in triglycerides, treatment with highly purified EPA was associated with significant reductions in levels of oxidized LDL, Lp-PLA₂, and hsCRP as compared to placebo. These antioxidant effects are consistent with the presently disclosed findings, which demonstrate EPA to be a potent and direct scavenger of free radicals. ROS and related oxidative damage have been implicated in the pathogenesis of various human chronic diseases. Due to its multiple conjugated double bonds, EPA has higher singlet oxygen quenching ability compared to vitamin E. EPA is expected to fully incorporate into the membrane bilayer, where it can exercise maximum free radical scavenging effects as shown in this study.

The antioxidant effects of EPA were enhanced in combination with the active metabolite of atorvastatin. According to primary pharmacokinetic studies, atorvastatin (parent) is extensively metabolized by hepatic cytochrome P450 to yield a number of active metabolites, which together reportedly account for approximately 70% of circulating HMG-CoA reductase inhibitory activity. This is in contrast to other statins like pravastatin and rosuvastatin that are not metabolized into active metabolites. Beyond their enzymatic effects on serum LDL-C levels, the active metabolites of atorvastatin may provide benefit by interfering with oxidative stress pathways. In a small study by Shishehbor et al. (Circulation, vol. 108(4), pages 426-431 (2003)) designed to evaluate the effects of atorvastatin therapy on markers of protein oxidation and inflammation, atorvastatin was found to significantly reduce circulating levels of chlorotyrosine, nitrotyrosine, and dityrosine, all of which act as surrogate markers for specific oxidative pathways upregulated in the atheroma. Interestingly, these effects were observed at a relatively low treatment dose (10 mg, administered for just 12 weeks) and were more significant than reductions in other inflammatory markers, including C-reactive protein. In a larger study by Tsimikas et al. (Circulation, vol. 110(11), pages 1406-1412 (2004)) involving 2,341 patients, treatment with a high dose of atorvastatin (80 mg) for 16 weeks caused a significant reduction in levels of oxidized lipids associated with all apoB100-containing lipid particles.

The ability of EPA to interfere with oxidative stress under conditions of hyperglycemia has important clinical implications. Levels of oxidized lipid, measured using monoclonal antibodies against oxLDL, have been shown by Ehara et al. (Circulation, vol. 103(15), pages 1955-1960 (2001)) to correlate with the severity of acute coronary syndromes and plaque instability. A more recent longitudinal investigation of 634 patients found that patients with baseline levels of thiobarbituric acid reactive substances (“TBARS”) in the highest quartile had significantly increased relative risk for major vascular events and procedures (Walter et al., J. Am. Coll. Cardiol., vol. 44(10), pages 1996-2002 (2004)). The predictive effect of TBARS was observed in a multivariate model adjusted for inflammatory markers (C-reactive protein, sICAM-1, IL-6) and other risk factors (age, LDL-C, high density lipoprotein cholesterol (HDL-C), total cholesterol, triglycerides, BMI, and blood pressure). These analyses indicated that TBARS had an independent effect on major vascular events and procedures. Similar predictive value was observed for LOOH in these same subjects in a follow-up study (Walter et al., J. Am. Coll. Cardiol., vol. 51(12), pages 1196-1202 (2008)). More recently, EPA treatment was associated with significant reductions in triglycerides along with reduced levels of markers of inflammation including hsCRP, Lp-PLA₂ and oxidized LDL, as compared to placebo, by Ballantyne et al. (Am. J. Cardiol., vol. 110(7), pages 984-992 (2012)) and Bays et al. (Am. J. Cardiovasc. Drugs, vol. 13(1), pages 37-46 (2013); Am. J. Cardiol., vol. 108(5), pages 682-690 (2011)).

In sum, pronounced changes in membrane lipid organization were observed with hyperglycemia, including the formation of cholesterol crystalline domains that correlate with an increase in lipid hydroperoxide (LOOH) formation (an intermediate product of oxidative lipid damage). Treatment of membranes with EPA, but not vitamin E, inhibited changes in membrane structure possibly due to potent chain-breaking antioxidant actions of the EPA molecules.

### Example 5

An experiment was conducted to compare the ability of EPA to prevent lipid hydroperoxide formation in model membranes treated with glucose to two other 20-carbon fatty acids: eicosanoic acid (“EA,” also referred to as arachidic acid, C20:0) and eicosatrienoic acid (“ETE,” C20:3, n−3). All MLV samples were subjected to time-dependent autoxidation by incubating at 37° C. in an uncovered, shaking water bath. This method allows lipid peroxidation to occur gradually without requiring the use of exogenous initiators. Small aliquots (5-100 μL) of each sample were removed, immediately following MLV preparation (0 hr) and after exposing samples to oxidative conditions for 72 or 96 hr, and combined with 1.0 mL of activated CHOD-iodide color reagent. Aliquot volume was reduced with each successive time point to ensure that spectrophotometric readings were within the optimal adsorption range. Test samples were covered and incubated in darkness at room temperature for at least 4 hr. Sample absorbances were then measured against a CHOD blank at 365 nm using a Beckman DU-640 spectrophotometer. The CHOD colorimetric assay is based on the oxidation of iodide (I⁻) by lipid hydroperoxide (LOOH) to form triiodide (I₃⁻), the quantity of which is directly proportional to the amount of LOOH present in the lipid sample. The molar absorptivity (E) of I₃⁻ is 2.46×10⁴ M⁻¹ cm⁻¹ at 365 nm.

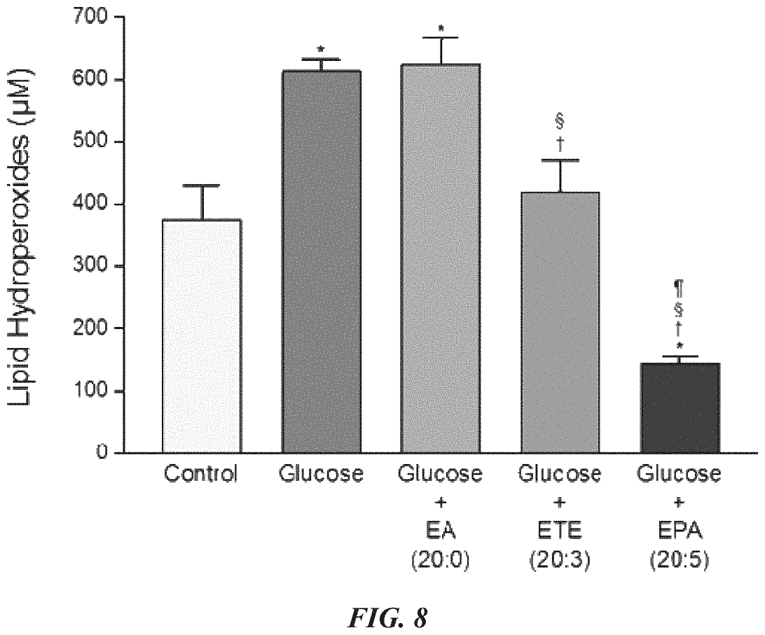

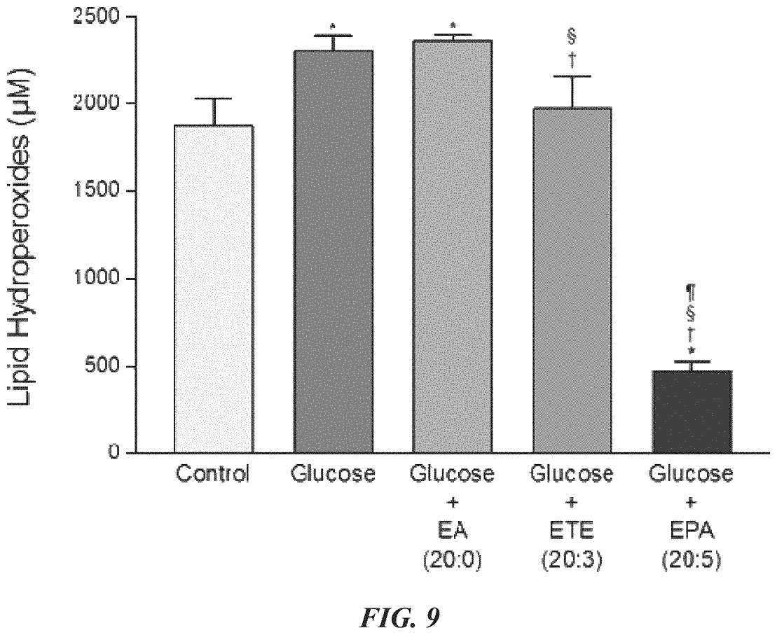

As shown in FIG. 8, model membranes treated with EPA had significantly less LOOH formation compared to model membranes treated with vehicle only (control), with glucose only, with glucose and EA, or with glucose and ETE. Values in FIG. 8 are mean±S.D. (N=6). After 96 hours (FIG. 9), the differences between EPA-treated model membranes and model membranes treated with vehicle only (control), with glucose only, with glucose and EA, or with glucose and ETE were even more pronounced. Values in FIG. 8 are mean±S.D. (N=6). *p<0.001 versus vehicle-treated control; ^{†}p<0.001 versus glucose-treated control; ^{§} p<0.001 versus EA; ^{¶}p<0.001 versus ETE (Student-Newman-Keuls multiple comparisons test; overall ANOVA: p<0.0001, F=148.57). *p<0.001 versus vehicle-treated control; ^{†}p<0.001 versus glucose-treated control. ^{§} p<0.001 versus EA; ^{¶}p<0.001 versus ETE (Student-Newman-Keuls multiple comparisons test; overall ANOVA: p<0.0001, F=248.73).

### Example 6

An experiment to study the antioxidant effect of EPA in small dense LDL (“sdLDL”) was performed. EPA was purchased from Sigma-Aldrich (Saint Louis, Mo.) and solubilized in ethanol to 1 mM under nitrogen atmosphere. Vitamin E (α-tocopherol) was purchased from Sigma-Aldrich and prepared in ethanol at 1.0 mM (ε=3.06×10⁴ M⁻¹ cm⁻¹ at 294 nm) just prior to experimental use. All test compounds were further diluted in ethanol or aqueous buffer as needed.

Venous blood was collected from healthy volunteers into vacutainer tubes containing sodium EDTA. Plasma was separated by centrifugation at 3000 g for 25 min at 4° C. and adjusted to a density of 1.020 g/mL with KBr. Triglyceride-rich lipoproteins (TGRL) and LDL fractions were then isolated by sequential centrifugation at 70,000 rpm at 4° C. in a Beckman LE-80 ultracentrifuge using a Beckman 50.4Ti rotor (Beckman Coulter, Inc., Fullerton, Calif.). The TGRL fraction (with a relative density of <1.020) was aspirated from the top of the centrifugate and discarded, leaving an LDL-enriched infranate (with a relative density of 1.020 to 1.063). The plasma LDL fraction was further fractionated into LDL1, LDL2, LDL3, and LDL4 (sdLDL) subfractions with relative densities of 1.020 to 1.035, 1.035 to 1.050, 1.050 to 1.063, and 1.063 to 1.075, respectively. The sdLDL subfraction was retained for additional experimentation. Plasma and lipoprotein fractions were maintained at 4° C. and protected from excessive exposure to light. Prior to oxidation experiments, EDTA was removed from the lipoprotein fraction using PD-10 desalting columns (GE Healthcare, Piscataway N.J.). After equilibration of the column with phosphate buffered saline (PBS), 2.5 mL of the lipoprotein fraction was loaded on the column and the flow-through discarded. The lipoprotein was then eluted with 3 mL of PBS. The LDL fractions were further diluted with PBS to obtain a 100 μg/mL apoB100 concentration.

LDL and sdLDL subfractions (0.6 mL each) were incubated with either 10 μL vehicle (ethanol) or 10 μL drug stock solution for 30 min to 1 hr at 37° C. Oxidation was initiated by the addition of 10 μM CuSO₄. After 2 hr, 100 μL aliquots were removed from each subfraction and combined with 1.0 mL thiobarbituric acid (0.5%), 10 μL trichloroacetic acid (5%), 10 μL BHT (5 mg/mL in methanol), and 10 μL EDTA (5 mM). Sample aliquots were incubated at 100° C. for 20 min and then assayed for the formation of thiobarbituric acid-reactive substances (TBARS), which have a molar absorptivity (E) value of 1.56×10⁵ M⁻¹ cm⁻¹ at 532 nm and are derived principally from the reaction of thiobarbituric acid with malondialdehyde (MDA), a reactive aldehyde produced by LDL oxidation. Sample TBARS concentrations were determined spectrophotometrically by measuring sample absorbances against a standard curve derived from the hydrolysis of 1,1,3,3-tetramethoxypropane.

Data are presented as mean±S.D. for (N) separate samples or treatment groups. Differences between groups were analyzed using the two-tailed, Student's t-test (for comparisons between only two groups) or ANOVA followed by Dunnett or Student-Newman-Keuls multiple comparisons post-hoc analysis (for comparisons between three or more groups). Only differences with probability values less than 0.05 were considered significant.

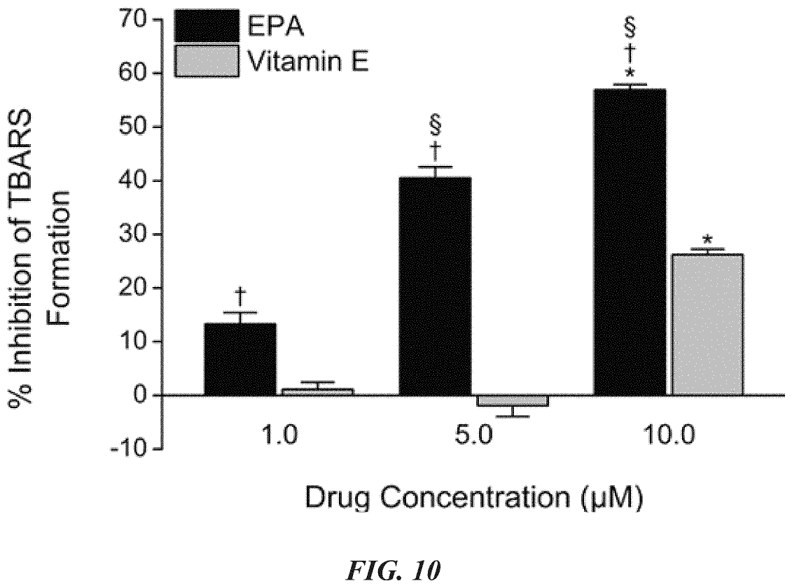

EPA has been shown in previous studies to inhibit lipid oxidation in phospholipid vesicles following exposure to high glucose levels and autoxidation (unpublished results). In this study these analyses were extended to human LDL and sdLDL fractions. As shown in FIG. 10, EPA inhibited sdLDL oxidation over a broad range of concentrations (1.0 μM to 10.0 μM). These concentrations are similar to the level of unesterified EPA measured in the plasma of humans (Cₘₐₓ:1.4 μg/mL, or ˜5 μM) with normally prescribed levels (e.g., 4 g/day) of EPA. At the lowest dose tested (1.0 μM), EPA reduced TBARS levels by 13% (p<0.001) compared to vehicle-treated controls; this inhibitory effect was dose-dependent and increased to 57% (p<0.001) at 10.0 μM. The comparative effects of vitamin E on sdLDL oxidation were also tested under identical conditions (FIG. 10). In contrast to EPA, vitamin E did not inhibit sdLDL oxidation except at the highest concentration (10.0 μM) where it reduced TBARS levels by 26% (p<0.05). These data indicate different interactions of these lipophilic, chain-breaking antioxidants with respect to sdLDL oxidation. Values in FIG. 10 are mean±S.D. (N=4). *p<0.05 versus control (Dunnett multiple comparisons test; overall ANOVA: p=0.0039, F=4.616); ^{†}p<0.0001 versus vitamin E treatment(s); ^{§} p<0.001 versus other EPA treatments (Student-Newman-Keuls multiple comparisons test; overall ANOVA: p<0.0001, F=764.91).

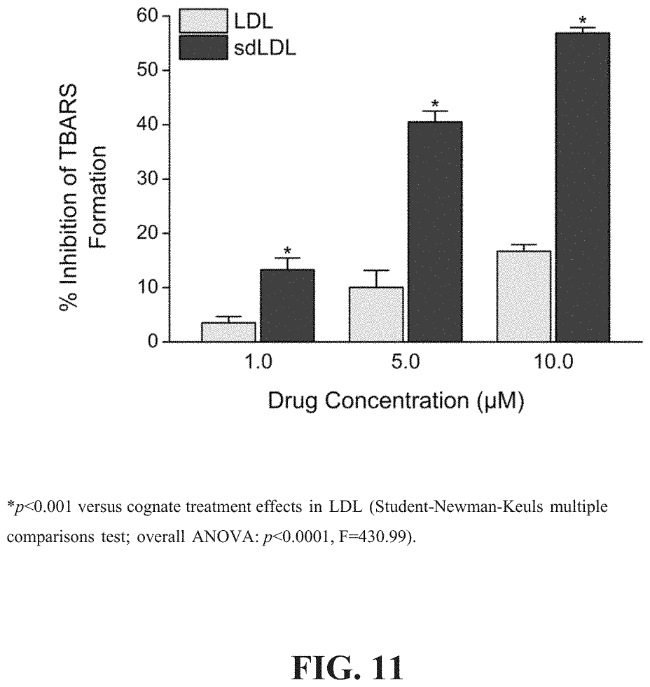

The antioxidant effects of EPA were further examined in unfractionated LDL particles (FIG. 11). LDL was isolated from human plasma, treated with vehicle, vitamin E, or EPA over a broad range of concentrations (1.0 μM to 10.0 μM) and examined for changes in lipid oxidation rates. EPA was found to generally inhibit LDL oxidation; however, the effects were less evident as compared to those observed for a similar number of sdLDL particles. At each concentration tested, the reduction in TBARS levels was several fold lower in the unfractionated LDL versus sdLDL (p<0.001). At the highest concentration tested (10.0 μM), EPA reduced TBARS levels by 17% and was similar to what was observed at the lowest dose tested (1.0 μM) in sdLDL. By contrast, vitamin E did not inhibit LDL oxidation at any concentration (data not shown). These data suggest that EPA has a disproportionately greater benefit in sdLDL as compared to the larger LDL species. Values in FIG. 11 are mean±S.D. (N=3-4).

### Example 7

An experiment to study the antioxidant effect of EPA in LDL and small dense LDL (“sdLDL”) was performed. LDL and sdLDL were isolated from the plasma of healthy volunteers by iodixanol density gradient ultracentrifugation, and adjusted to a final apolipoprotein concentration of 2 mg/mL. Sample aliquots (100 μg apolipoprotein each) were incubated with EPA, fenofibrate, gemfibrozil, or niacin (each at 1.0, 5.0, or 10.0 μM) for 30 min at 37° C. Fenofibrate, gemfibrozil, and niacin were purchased from Sigma-Aldrich. Lipid oxidation was initiated with 10 μM CuSO₄ and monitored by the colorimetric detection of thiobarbituric acid reactive substances (TBARS) for 1 hr. Vitamin E was also incubated with sample aliquots as a positive control. For comparison, the active o-hydroxy metabolite of atorvastatin (ATM, Toronto Research Chemicals, North York, Ontario, Canada), alone or in combination with EPA or DHA, was also incubated with sample aliquots.

Results: sdLDL

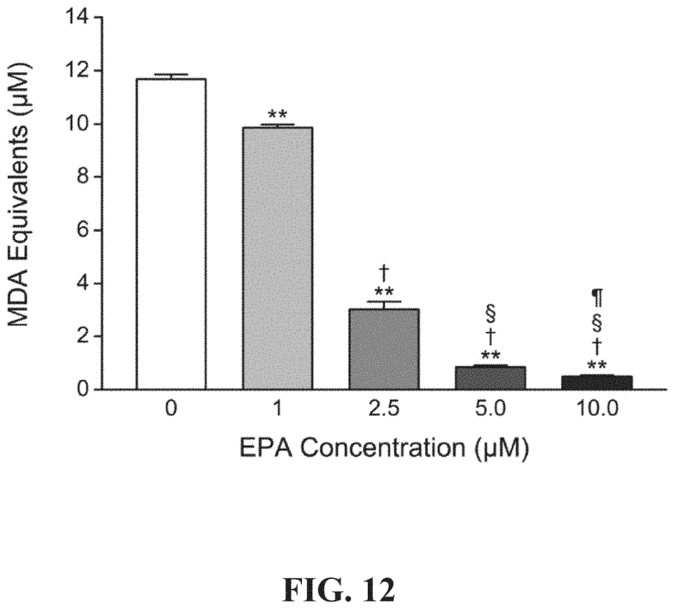

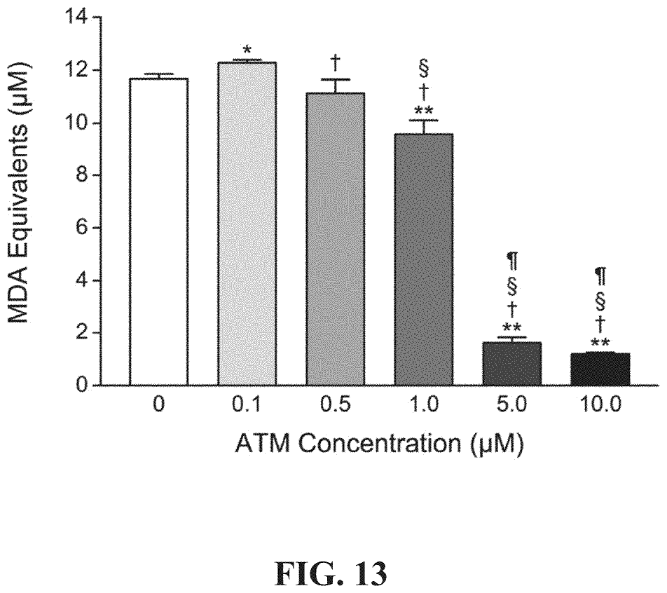

As shown in FIG. 12, EPA significantly and reproducibly inhibited sdLDL oxidation in a dose-dependent manner with an IC₅₀ of ˜2.0 μM. At the highest treatment concentration (10 μM), EPA inhibited TBARS formation by 93±2% (p<0.001). Inhibition was significant and reproducible with EPA by 19±8% (p<0.001) even at the lowest dose of 1.0 μM. The IC₅₀ for EPA was calculated to be approximately 2 μM. ATM also showed a dose-dependent inhibition of sdLDL oxidation with a significant effect of 18±5% (p<0.001) beginning at 1.0 μM as shown in FIG. 13. At the highest dose, the inhibition with ATM was 90±2% (p<0.001).

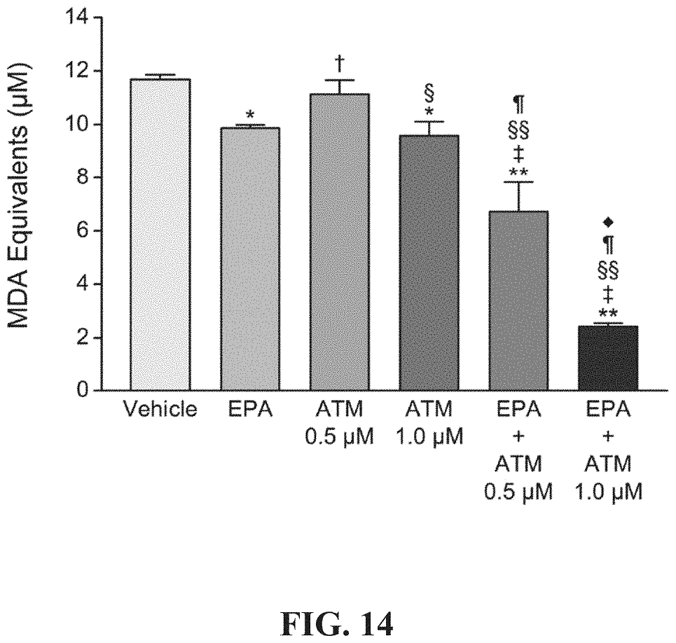

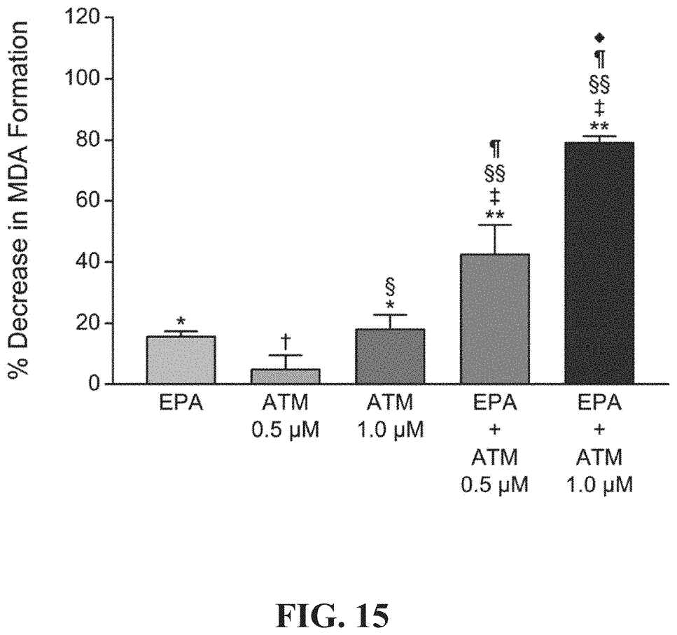

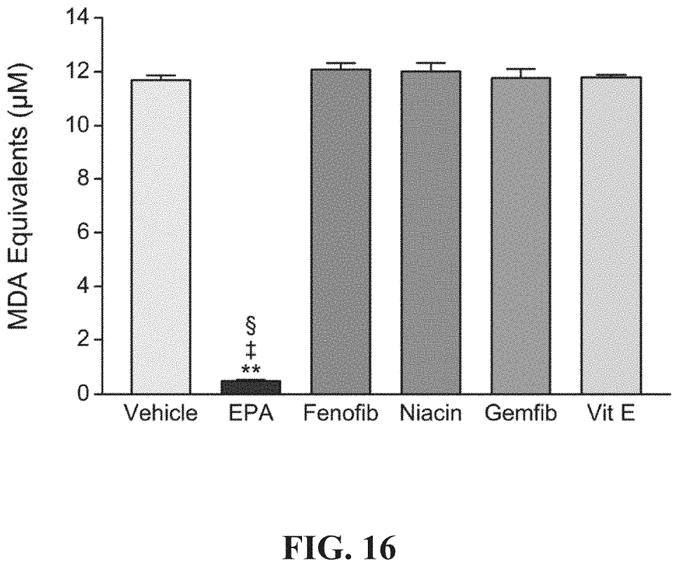

Pretreatment with a combination of EPA and ATM followed by oxidative stress reduced sdLDL oxidation to an extent that was not observed with their individual treatments at equimolar concentrations. (FIGS. 14-15). Individual EPA and ATM treatments inhibited sdLDL oxidation at 1.0 μM by 19±8% and 18±5%, respectively, while the combination inhibited sdLDL oxidation by 75±8% as compared to vehicle (p<0.001). The effects of the combination at a lower dose of ATM (0.5 μM) resulted in inhibition of only 5±5% (ATM alone) but 57±9% (p<0.001) in combination with EPA (1.0 μM). The inhibition with the ATM/EPA combination was much greater than the sum of their separate effects at the concentrations we tested in this study. As shown in FIG. 16, fenofibrate, gemfibrozil, niacin, and vitamin E had no significant effect on sdLDL oxidation as compared to vehicle-treated controls.

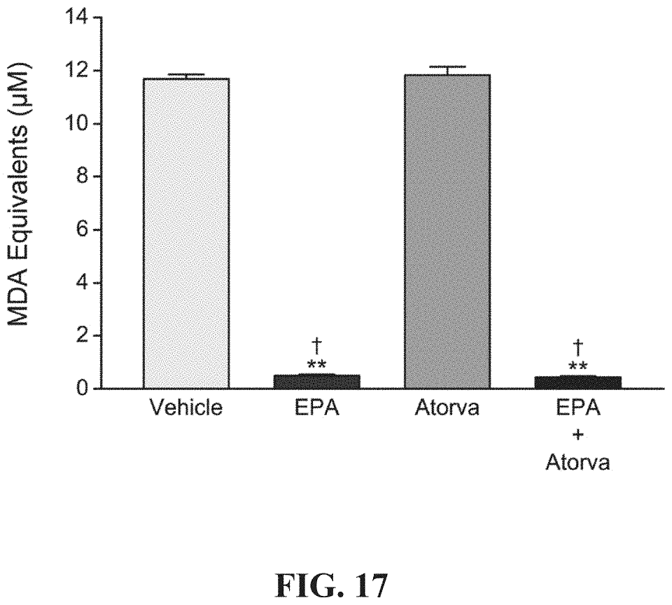

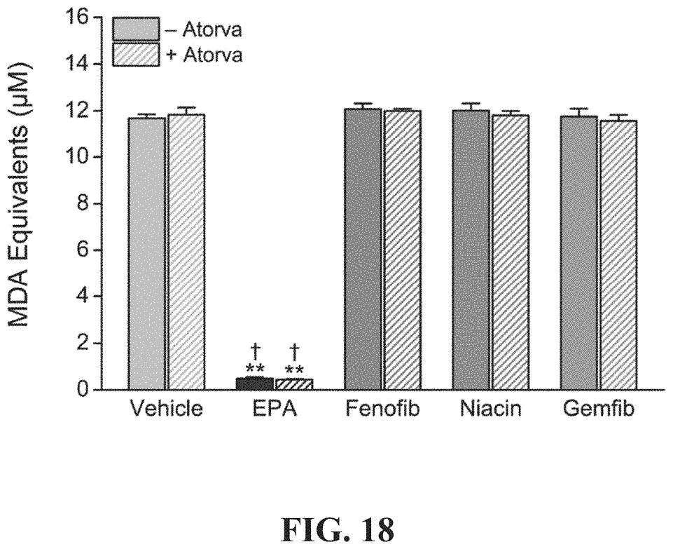

The individual and combined effects of EPA and atorvastatin (parent) pretreatment of sdLDL at 10.0 μM followed by stimulation of oxidative stress were tested. As shown in FIG. 17, the combination of EPA and atorvastatin reduced sdLDL oxidation to the same extent that was observed with EPA alone. Separately, atorvastatin did not have any significant antioxidant activity. The individual EPA and atorvastatin treatments inhibited sdLDL oxidation at 10.0 μM by 92±8% and 0.1±7%, respectively, while the combination inhibited sdLDL oxidation by 91±8% as compared to vehicle (p<0.001). The inhibition of sdLDL oxidation with EPA/atorvastatin combination was thus similar to that observed separately for EPA alone and not observed in combination with other TG-lowering agents (FIG. 18).

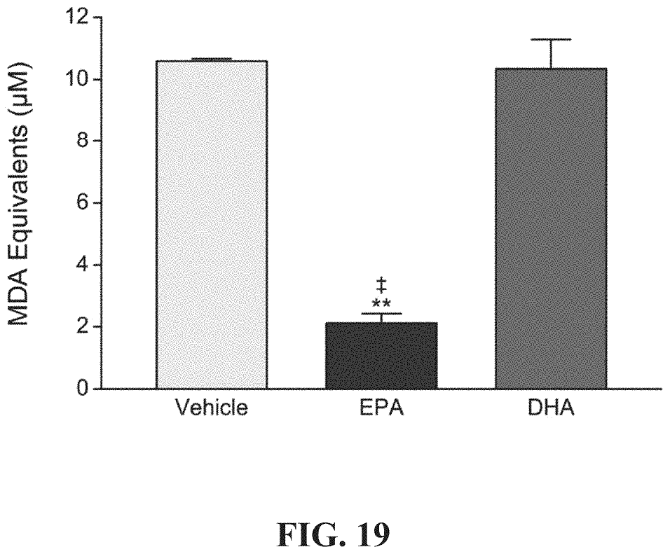

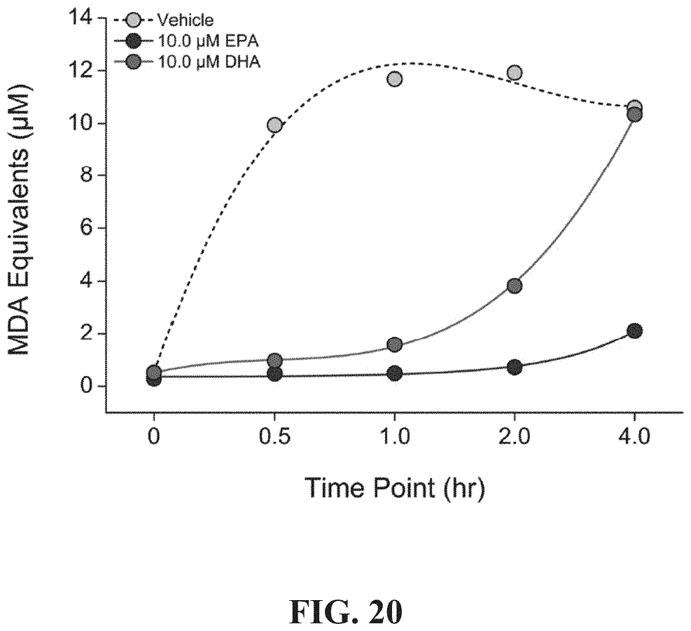

The antioxidant activity of EPA to DHA was tested by pretreatment at an equimolar concentration (10.0 μM) followed by stimulation of oxidative stress. As shown in FIG. 19, only EPA significantly inhibited sdLDL oxidation after 4 hours as compared to vehicle or DHA (p<0.001). The differences in activity between EPA and DHA became more apparent during the time course of the experiment with significant differences as early as 1 hour (FIG. 20).

Results: Unfractionated LDL

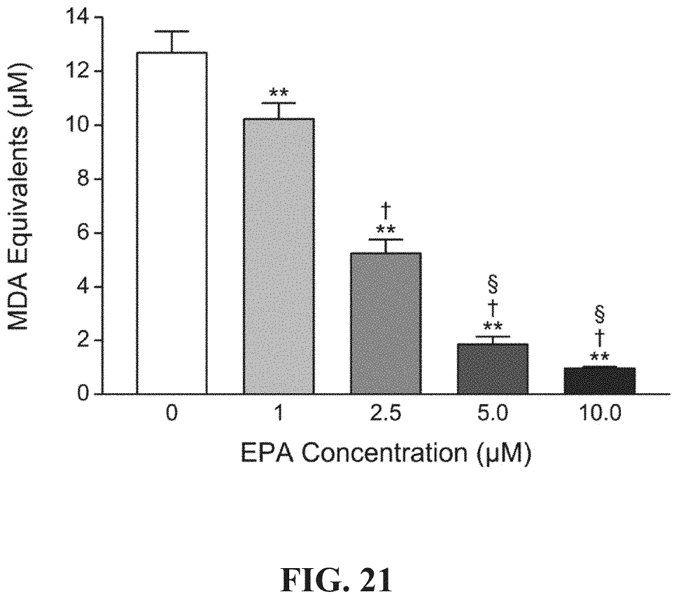

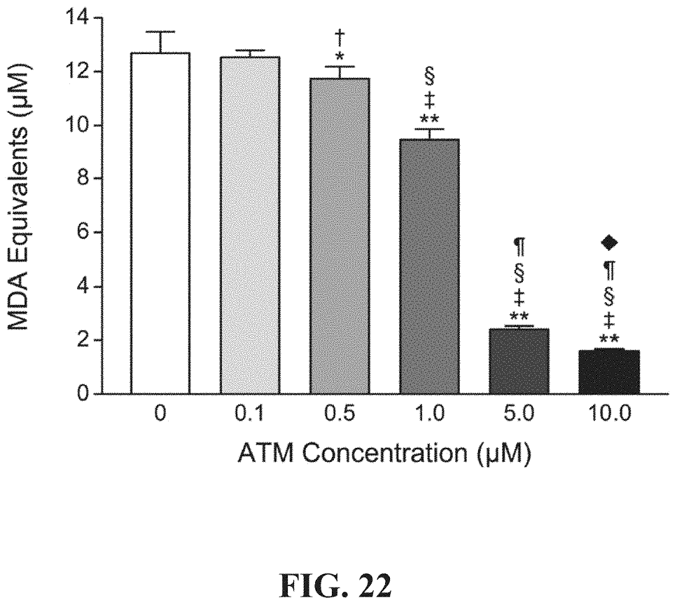

As shown in FIG. 21, EPA also significantly and reproducibly inhibited unfractionated LDL oxidation in a dose-dependent manner. At the highest treatment concentration (10 μM), EPA inhibited TBARS formation by >90% (p<0.001) compared to vehicle-treated control. Inhibition was significant with EPA even at the lowest dose (1 μM). As shown in FIG. 22, ATM also showed a significant and dose-dependent inhibition of unfractionated LDL oxidation beginning at 1.0 μM.

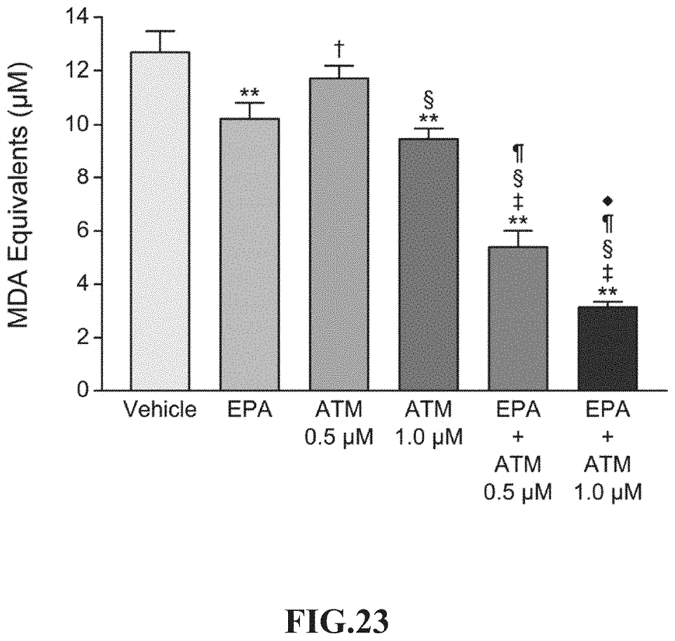

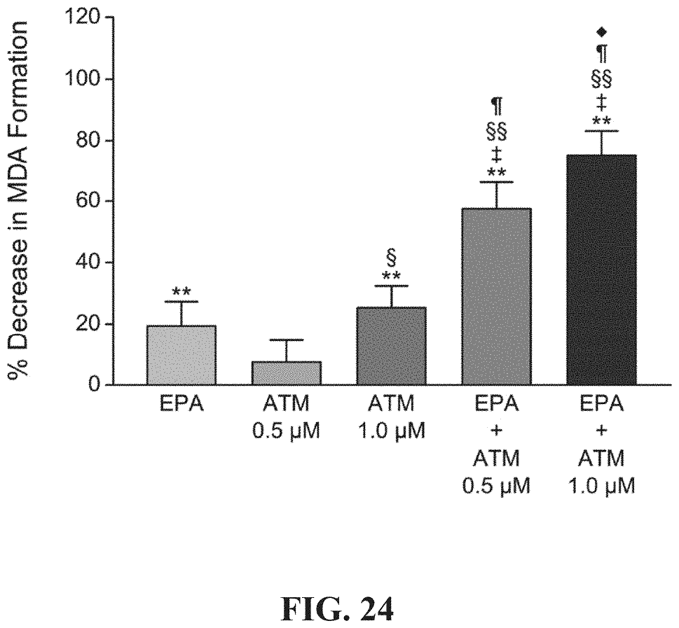

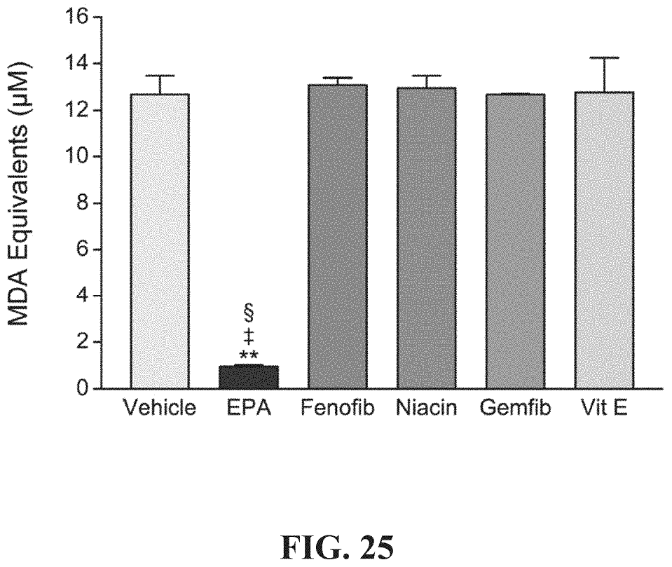

Pretreatment with a combination of EPA and ATM followed by oxidative stress reduced unfractionated LDL oxidation to an extent that was not observed with their individual treatments at equimolar concentrations. (FIGS. 23-24). The inhibition with the ATM/EPA combination was much greater than the sum of their separate effects at the concentrations we tested in this study. As shown in FIG. 25, fenofibrate, gemfibrozil, niacin, and vitamin E had no significant effect on unfractionated LDL oxidation as compared to vehicle-treated controls.

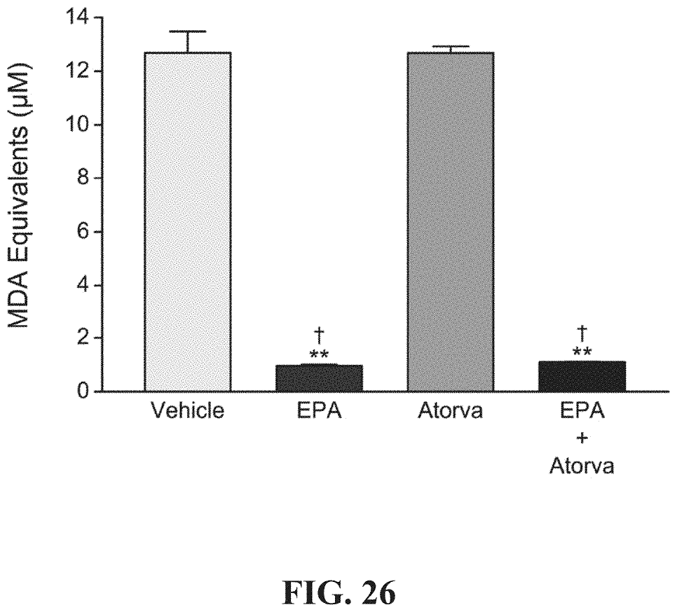

The individual and combined effects of EPA and atorvastatin (parent) pretreatment of unfractionated LDL at 10.0 μM followed by stimulation of oxidative stress were tested. As shown in FIG. 26, the combination of EPA and atorvastatin reduced unfractionated LDL oxidation to the same significant extent (p<0.001 vs. vehicle or atorvastatin alone) that was observed with EPA alone. Separately, atorvastatin did not have any significant antioxidant activity.

These data show that EPA effectively blocks sdLDL and LDL oxidation at pharmacologic concentrations in vitro and in a manner that could not be reproduced with vitamin E or other TG-lowering agents. ATM had similar activity that was enhanced in combination with EPA.

### Example 8

### Materials

Human umbilical vein endothelial cells (HUVECs) were isolated into primary cultures from female donors by Clonetics (San Diego, Calif.) and purchased as proliferating cells. All cell culture donors were healthy, with no pregnancy or prenatal complications. The cultured cells were incubated in 95% air/5% CO₂ at 37° C. and passaged by an enzymatic (trypsin) procedure. The confluent cells (4 to 5×10⁵ cells/35 mm dish) were placed with minimum essential medium containing 3 mM L-arginine and 0.1 mM BH4 [(6R)-5,6,7,8-tetrahydrobiopterin]. Before experimental use, the cells (from second or third passage) were rinsed twice with Tyrode-HEPES buffer with 1.8 mM CaCl₂.

The omega-3 fatty acids (O3FAs) cis-5,8,11,14,17-eicosapentaenoic acid (EPA) and docosahexaenoic acid (DHA) were purchased from Sigma-Aldrich (St. Louis, Mo.) and prepared initially at 11 mM in redistilled ethanol. Primary and secondary O3FA stock solutions were prepared and stored under argon at −20° C. Ortho (o)-hydroxy atorvastatin metabolite (ATM) was synthesized and purchased from Toronto Research Chemicals (North York, Ontario, Canada) and solubilized in ethanol at 1 mM; subsequent dilutions were prepared in ethanol or aqueous buffer as needed.

### LDL Isolation

We tested the effects of EPA treatment in the absence and presence of ATM in HUVECs following exposure to oxidized LDL (oxLDL). The oxLDL causes eNOS uncoupling and endothelial dysfunction to reproduce disease-like conditions.25 Venous blood from healthy normolipidemic volunteers was collected into Na-EDTA (1 mg/mL blood) vacuum tubes after a 12-hour fast. Plasma was immediately separated by centrifugation at 3,000 g for 10 minutes at 4° C. LDL (6=1.020 to 1.063 g/mL) was separated from freshly drawn plasma by preparative ultracentrifugation with a Beckman ultracentrifuge equipped with an SW-41 rotor. The density of plasma was adjusted to 1.020 g/mL with sodium chloride solution, the plasma was centrifuged at 150,000 g for 24 hours, and the chylomicron-rich layers were discarded. The remaining fraction, after adjustment of density at 1.063 g/mL with potassium bromide, was centrifuged at 150,000 g for 24 hours to isolate LDL from the HDL fraction. The purified LDL was dialyzed for 96 hours against PBS containing 0.3 mM EDTA at 4° C., then stored at 4° C.

### LDL Oxidation

A sample of LDL was dialyzed against Tris/NaCl Buffer (50 mM Tris in 0.15 M NaCl, pH 8.0) to remove the EDTA. Tris-NaCl buffer was added to the dialyzed LDL to adjust the protein concentration to 30 mg/mL. A 1-mL aliquot of 20 μM CuSO₄ was added to 1 mL of dialyzed normal LDL. Oxidation at 37° C. was followed spectrophotometrically (234 nm) over a period of 24 hours until oxidation was complete. The oxLDL was then dialyzed at 4° C. with 4 L Tris buffer, filtered with a 0.22 m filter, and stored under nitrogen at 4° C.

Oxidation was monitored by the use of measurements of TBARS. Briefly, LDL was incubated with thiobarbituric acid (0.5 wt/vol, in H₂SO₄, 50 mM) for 30 minutes at 100° C. The solution then was centrifuged for 5 minutes, and the difference in absorbency at 532 and 580 nm was calculated. TBARS concentration was determined as malondialdehyde (MDA) equivalents with the use of an MDA standard curve.

### NO and ONOO⁻ Nanosensors

Concurrent measurements of NO and ONOO⁻ were performed with tandem electrochemical nanosensors combined into one working unit with a total diameter of 200-400 nm. Their design was based on previously developed and chemically modified carbon-fiber technology. Each of the nanosensors was made by depositing a sensing material on the tip of a carbon fiber (length 4 to 5 m, diameter 100-200 nm). The fibers were sealed with nonconductive epoxy and electrically connected to copper wires with conductive silver epoxy. Conductive films of polymeric Ni(II) tetrakis (3-methoxy-4-hydroxyphenyl) porphyrin and Mn(III) [2.2] paracyclophanylporphyrin were used for the NO and ONOO⁻ sensors, respectively.

The amperometric method (with a response time of 0.1 ms) provides a quantitative signal (current) that is directly proportional to changes (from basal levels) in NO or ONOO⁻ concentration. Amperometric measurements were performed with a Gamry III double-channel potentiostat. Basal NO or ONOO⁻ levels were measured by differential pulse voltammetry in separate experiments.

All measurements of NO and ONOO⁻ were performed on intact endothelial cells. The NO/ONOO⁻ nanosensor module was positioned 5±2 m from the surface of each individual endothelial cell using a computer-controlled M3301 micromanipulator (x-y-z resolution of 0.2 m) and microscope (both from World Precision Instruments, Berlin, Germany) fitted with a CD camera. After establishing a background current, EPA, in the absence and presence of ATM, was added to the cells. Rapid changes in current (proportional to the molar concentrations of NO or ONOO⁻ released) were observed after the addition of Cal and were monitored continuously.

Treatment with oxLDL, EPA and Statins

Primary human umbilical vein endothelial cells (HUVECs) were incubated with vehicle or oxidized human low density lipoprotein (oxLDL) for 20 min prior to treatment with EPA and/or ATM. After this incubation period, the cells were treated with vehicle or with 10 μM EPA in the absence or presence of 1.0 μM ATM for 1 hour. Endothelial basal media was used for all the treatments. Controls were supplied with an equivalent volume of endothelial basal media.

Statistical Analyses

Data are presented as mean±S.D. for (N) separate samples or experiments. Differences between groups were analyzed using the two-tailed, Student t-test (for comparisons between only two groups) or ANOVA followed by Student-Newman-Keuls multiple comparisons post-hoc analysis (for comparisons between three or more groups). Only differences with probability values less than 0.05 were considered significant.

### Results

A summary of all measurements of NO and ONOO⁻ release in HUVECs, along with the NO/ONOO⁻ ratios, is included in Table 1.

TABLE 1
NO and ONOO⁻ Release from HUVECs.

| | | | NO/ONOO⁻ |
|---|---|---|---|
| Treatment | NO | ONOO⁻ | Ratio |
| Vehicle | 376 ± 85 | 239 ± 41 | 1.57 ± 0.45 |
| oxLDL + Vehicle | 292 ± 53 | 246 ± 26 | 1.19 ± 0.25 |
| oxLDL + EPA | 423 ± 163 | 189 ± 25 | 2.24 ± 0.91 |
| oxLDL + ATM | 480 ± 75 | 196 ± 34 | 2.45 ± 0.57 |
| oxLDL + EPA + ATM | 590 ± 190 | 172 ± 12 | 3.43 ± 1.13 |
| oxLDL + DHA | 567 ± 103 | 167 ± 18 | 3.39 ± 0.72 |
| oxLDL + DHA + ATM | 462 ± 117 | 133 ± 28 | 3.48 ± 1.14 |
| oxLDL + Fenofibrate | 327 ± 187 | 299 ± 24 | 1.09 ± 0.63 |
| oxLDL + Fenofibrate + ATM | 471 ± 106 | 243 ± 30 | 1.94 ± 0.50 |
| oxLDL + Niacin | 431 ± 100 | 215 ± 25 | 2.00 ± 0.52 |
| oxLDL + Niacin + ATM | 436 ± 118 | 198 ± 28 | 2.20 ± 0.67 |
| oxLDL + Gemfibrozil | 192 ± 34 | 266 ± 34 | 0.72 ± 0.16 |
| oxLDL + Gemfibrozil + ATM | 418 ± 85 | 214 ± 31 | 1.95 ± 0.49 |
| EPA + oxLDL | 663 ± 50 | 247 ± 17 | 2.68 ± 0.28 |

Values in Table 1 are reported as mean +/− S.D. (N = 3-16).

In comparison to vehicle, EPA, DHA, and niacin separately have beneficial effects on the release of NO, ONOO⁻ and their ratio. By contrast, fenofibrate treatment produced an increase in NO release but caused a disproportionate and detrimental increase in ONOO⁻ release, thereby reducing the ratio of NO to ONOO⁻. Gemfibrozil treatment reduced both NO release and the NO to ONOO⁻ ratio.

We also tested the effects of EPA and other TG-lowering agents, along with DHA, to ATM alone and in combination as reviewed in Table 1. We observed that EPA+ATM treatment was associated with a similar benefit in the relative release of NO and ONOO⁻. As a result, treatment with EPA+ATM continued to demonstrate an overall benefit in the NO to ONOO⁻ ratio. Treatment with DHA+ATM also produced an overall benefit in the NO to ONOO⁻ ratio, but this was primarily attributed to a decrease in ONOO⁻ release but no improvement in NO production. This is in contrast to the effects with EPA and even DHA monotherapy. The combination of ATM+niacin resulted in its relative loss in benefit as compared to monotherapy. As compared to ATM alone, fenofibrate+ATM treatment caused a loss in any minimal benefit in NO and even preserved the detrimental effects on ONOO⁻ release. This resulted in an adverse effect on the NO to ONOO⁻ ratio. Finally, gemfibrozil+ATM treatment had a minimal effect on ONOO⁻ release and a detrimental effect on NO release, along with the NO to ONOO⁻ ratio.

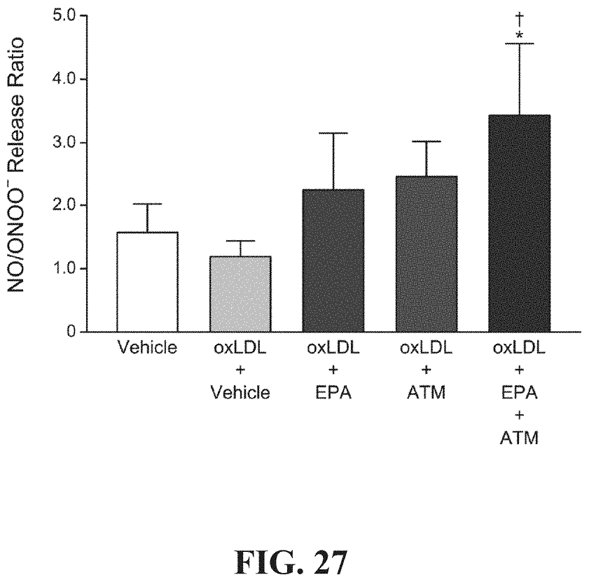

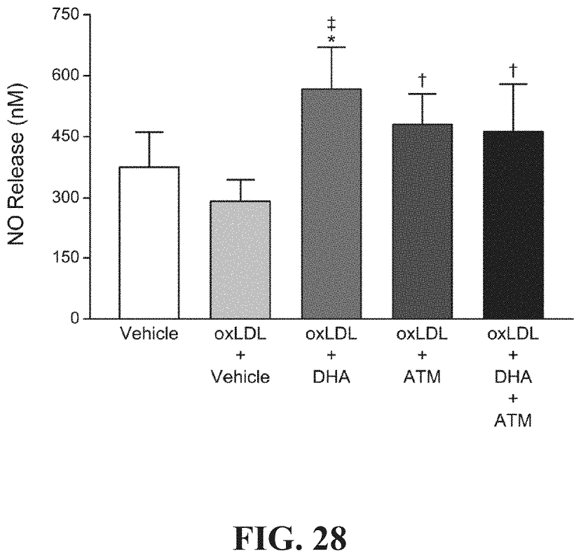

As shown in FIG. 27, exposure of HUVECs to oxLDL decreased NO release from HUVECs by 22% (376±85 nM to 292±53 nM) while increasing ONOO⁻ release as compared to untreated cells. In ECs exposed to oxLDL, treatment with EPA and ATM separately increased NO release by 45% (423±163 nM) and 64% (480±75 nM), respectively. When combined, treatment with EPA+ATM increased NO release by 200% (590±190 nM) as compared to vehicle (p<0.01) (FIG. 27). The improvement in NO release with the EPA+ATM combination was also greater than cells treated with non-oxidized LDL (p<0.05). By contrast, the combination of DHA with ATM did not improve NO release as compared to ATM or DHA separately (FIG. 28).

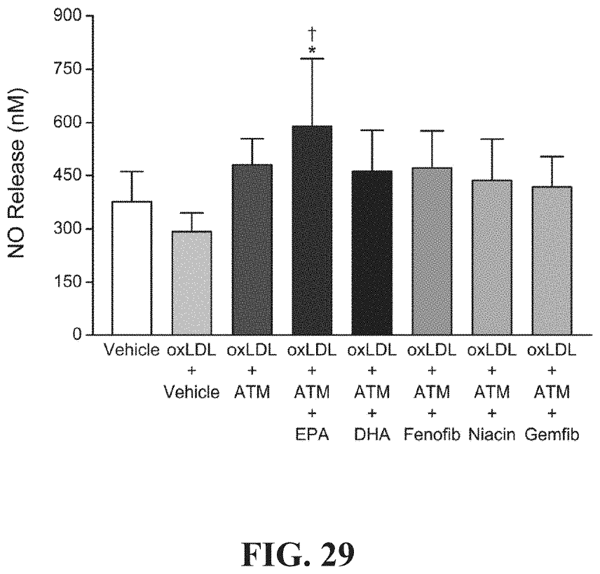

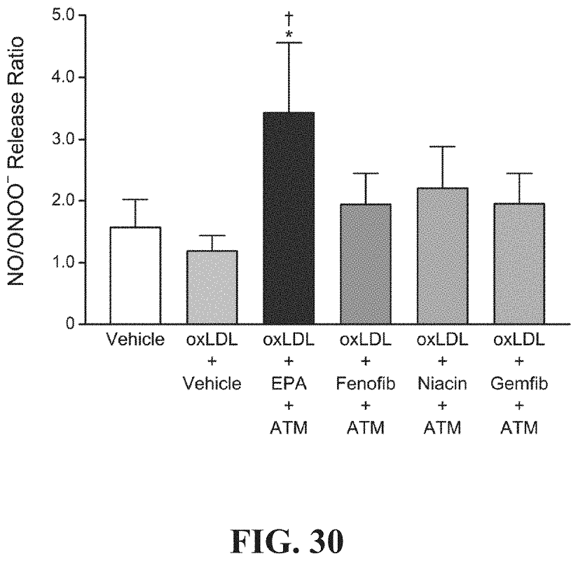

When comparing all the agents, the improvement in NO with EPA was not reproduced with DHA or the other TG-lowering agents when combined with ATM (FIG. 29). Furthermore, the NO/ONOO⁻ ratio, an indicator of normal EC function, increased by approximately 3-fold with the EPA and ATM combination treatment as compared to control treatments (p<0.05). The improvement in the NO/ONOO⁻ ratio was not observed with other TG-lowering agents as compared to EPA (FIG. 30).

### Example 9

The objective of this study is to compare dose-dependent effects of EPA, EPA-docosahexaenoic acid (DHA) combination, DHA alone, glycyrrhizin, alpha-linolenic acid (ALA), docosapentaenoic acid (DPA), arachidonic acid (AA), fenofibrate, gemfibrozil and niacin on membrane cholesterol domains that model atherosclerosis.

Membrane cholesterol domains are associated with atheroma development and formation of toxic cholesterol crystals. Agents that reduce such membrane domains would be expected to have important therapeutic benefits.

### Materials

The omega-3 fatty acids, eicosapentaenoic acid (EPA), docosahexaenoic acid (DHA), glycyrrhizin, docosapentaenoic acid (DPA), alpha-linolenic acid (ALA) and the omega-6 fatty acid arachidonic acid (AA) were purchased from Sigma-Aldrich (Saint Louis, Mo.) and solubilized in redistilled ethanol to 500 μM under nitrogen atmosphere. The fatty acid stock solutions were stored at −20° C. until use. Fenofibrate, gemfibrozil and nicotinic acid were purchased from Toronto Research Chemicals (North York, Ontario, Canada) and solubilized in ethanol at 1 mM; subsequent dilutions were prepared in ethanol or aqueous buffer as needed.

### Methods

Multilamellar vesicles (MLVs) were prepared as binary mixtures that include palmitoyloleoylphosphatidylcholine (POPC) (1.0 mg total phospholipid per sample) and cholesterol at an elevated cholesterol-to-phospholipid (C/P) mole ratio of 1.5:1. Component lipids (in chloroform) were transferred to 13×100 mm borosilicate culture tubes and then combined with vehicle (ethanol) or an equal volume of EPA, EPA+DHA, DHA, DPA, ALA, AA stock solutions or with other TG-lowering agents (fenofibrate, gemfibrozil, niacin), adjusted to achieve desired treatment concentrations (total drug-to-phospholipid mole ratio of 1:30), and incubated with the test agent during removal of solvent. Samples were shell-dried under nitrogen gas and placed under vacuum for 3 hours to remove residual solvent at room temperature. After desiccation, each sample was resuspended in saline buffer (0.5 mM HEPES, 154 mM NaCl, pH 7.3) to yield a final phospholipid concentration of 2.5 mg/mL. Lipid suspensions were then vortexed for 3 minutes at ambient temperature to form MLVs.

Membrane lipid vesicles were oriented for x-ray diffraction analysis as described by others (e.g., Herbette et al., Biophys. J., vol. 20(2), pages 245-272 (1977)). Briefly, a 100 μL aliquot of each MLV preparation (containing 250 μg MLV) was transferred to a Lucite® sedimentation cell fitted with an aluminum foil substrate designed to collect MLVs into a single membrane pellet upon centrifugation. Samples were then loaded into a Sorvall AH-629 swinging bucket rotor (DuPont Corp., Wilmington, Del.) and centrifuged at 35,000 g, 5° C., for 90 minutes.

After centrifugal orientation, sample supernatants were aspirated and aluminum foil substrates, each supporting a single membrane pellet, were removed from the sedimentation cells. Sample pellets were dried for 5-10 minutes at ambient conditions, mounted onto curved glass supports, and placed in hermetically-sealed, brass or glass containers (for immediate analysis or temporary storage, respectively). All x-ray diffraction experiments were conducted at 20° C., 74% relative humidity. The latter was established by exposing membrane samples to saturated solutions of L-(+)-tartaric acid (K₂C₄H₄O₆.½H₂O) for at least 1 hour prior to experimental analysis.

Oriented membrane samples were aligned at grazing incidence with respect to a collimated, mono-chromatic CuK_{α} x-ray beam (K_{α1} and K_{α2} unresolved; λ=1.54 Å) produced by a Rigaku Rotaflex RU-200, high-brilliance microfocus generator (Rigaku-MSC, The Woodlands, Tex.) as previously described (Mason et al., Biophys. J., vol. 55(4), pages 769-778 (1989)). The samples were initially subjected to x-ray diffraction analysis at least 12 hours after formation of the membrane pellets and retested for up to several days. Analytical x-rays were generated by electron bombardment of a rotating copper anode and are filtered through a thin nickel foil to provide monochromatic radiation (X=1.54 Å). Collimation of the x-ray beam was achieved using a single Franks mirror. Diffraction data were collected on a one-dimensional, position-sensitive electron detector (Hecus X-ray Systems, Graz, Austria) at a sample-to-detector distance of 150 mm from the sample site.

The d-space for any given membrane multi bilayer is a measurement of the unit cell periodicity of the membrane lipid bilayer (e.g., the distance from the center of one lipid bilayer to the next including surface hydration), and is calculated from Bragg's Law, hλ=2d sin θ, where h is the diffraction order, λ is the wavelength of the x-ray radiation (1.54 Å), d is the membrane lipid bilayer unit cell periodicity, and θ is the Bragg angle equal to one-half the angle between the incident beam and scattered beam.

The presence of cholesterol domains in a given membrane sample results in the production of distinct Bragg (diffraction) peaks having singular periodicity values of 34 and 17 Å (typically referred to as first- and second-order cholesterol domain peaks). Under the specific temperature and relative humidity conditions established for these experiments, the second-order, 17 Å cholesterol domain peak was well-delineated from other, neighboring cholesterol and phospholipid diffraction peaks and was thus used to quantitate relative cholesterol domain peak intensity. Routines written in Origin 8.6 (OriginLab Corporation, Northampton, Mass.) were used to determine total peak area (associated with all diffraction peaks in a given pattern) against which the second-order cholesterol domain peak was normalized.

### Statistical Analyses

The raw data collected in this study was normalized using internal. Data were presented as mean±S.E.M. for (N) separate samples or treatment groups. Differences between groups were analyzed using the two-tailed, Student's t-test (for comparisons between only two groups) or ANOVA followed by Dunnett or Student-Newman-Keuls multiple comparisons post-hoc analysis (for comparisons between three or more groups). Alpha error was set to 0.05 in this study.

### Results

Comparative effects of eicosapentaenoic acid (EPA), docosahexaenoic acid (DHA), EPA-DHA combination treatment, fenofibrate (Fenofib), nicotinic acid (Niacin), gemfibrozil (Gemfib), glycyrrhizin (Glyc), arachidonic acid (AA), α-linolenic acid (ALA), and docosapentaenoic acid (DPA) on normalized cholesterol domain peak intensity as measured in model membrane prepared as binary mixtures of POPC and cholesterol at a 1.5:1 C/P mole ratio are reviewed in Table 2.

TABLE 2
Cholesterol Domain Peak Intensities for EPA and Other Agents.

| | Relative Cholesterol | % change | |
|---|---|---|---|
| Treatment | Domain Preak Intensity | Vs control | P values |
| Vehicle | 42.3 ± 4.3 | — | — |
| EPA (20:5; n-3)* | 14.6 ± 5.0 | −65.5 ± 17.1 | 0.0139 |
| DHA (22:6; n-3)* | 37.6 ± 2.4 | −11.1 ± 11.7 | 0.3975 |
| EPA + DBA* | 29.8 ± 7.6 | −29.6 ± 20.9 | 0.2279 |
| Fenofib* | 41.7 ± 6.8 | −1.4 ± 19.1 | 0.9451 |
| Niacin* | 27.5 ± 12.2 | −35.0 ± 30.8 | 0.3157 |
| Gemfib* | 46.9 ± 12.6 | 10.9 ± 43.3 | 0.8122 |
| Glyc* | 19.1 ± 1.2 | −54.9 ± 12.3 | 0.0002 |
| AA (20:4; n-6) | 22.5 ± 4.3 | −46.8 ± 15.2 | 0.0316 |
| ALA (18:3; n-3) | 23.2 ± 0.6 | −45.2 ± 11.3 | 0.0122 |
| DPA (22:5; n-3) | 33.5 ± 2.7 | −20.8 ± 12.2 | 0.1587 |

Values are reported as mean ± SEM (N = 3-6).
*P values were calculated against vehicle-treated controls using an unpaired, two-tailed Student's t-test.
Values within parentheses after fatty acids denote carbon length and number of unsaturated bonds; omega-3 or -6 fatty acid classification.

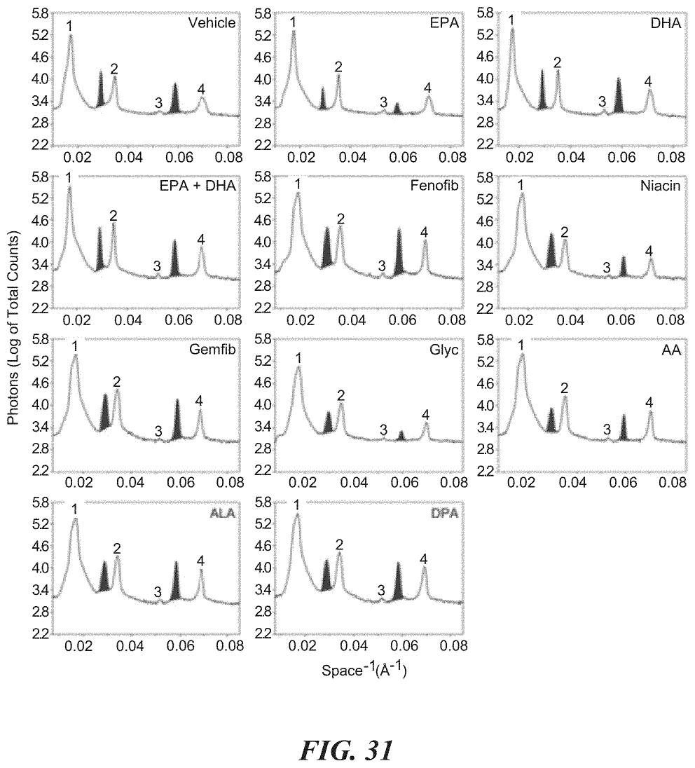

The percent changes in the area of the peaks were compared to vehicle control treatment and representative diffraction patterns for each of the tested agents were calculated as shown in FIG. 31.

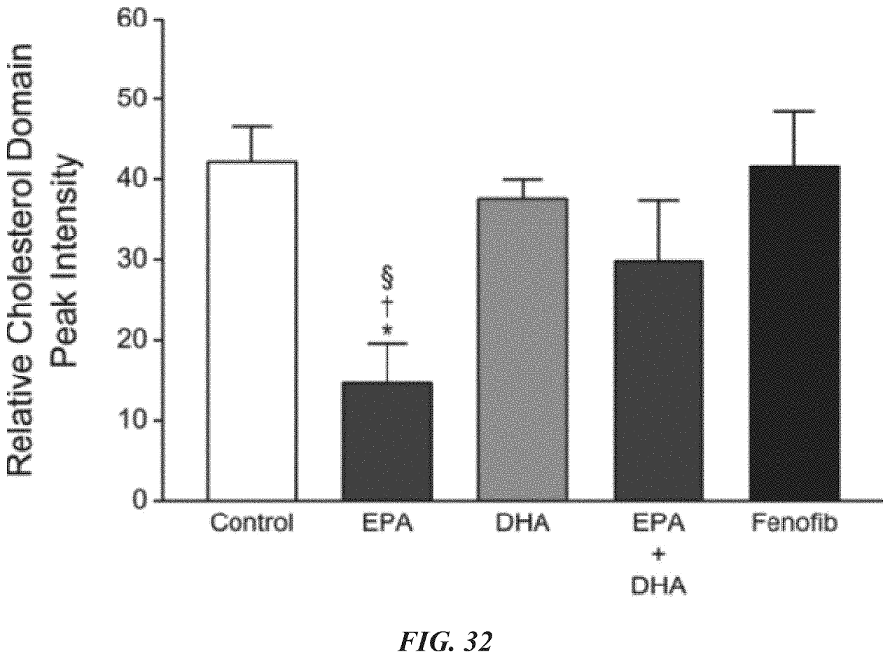

EPA treatment caused a pronounced and significant (p<0.05) reduction in cholesterol domain peaks by 65% (14.6±5.0) as compared to vehicle treatment (42.3±4.3) as shown in FIG. 32.

Samples treated with DHA did not reduce cholesterol domains (37.6±2.4); the integrated peak areas were significantly larger than those observed for EPA (p<0.05). The combination of EPA with DHA showed an intermediate reduction in domain formation of 30% (29.8±7.6), consistent with a lower level of EPA. The difference in domain size with the EPA+DHA combination at a 1:1 molar ratio was a non-significant −29.6% (29.8±7.6) compared to vehicle or other treatments (FIG. 32). Relative cholesterol peak intensity values were derived by integrating the second-order cholesterol domain peak and normalizing to total phospholipid peak area associated with a given diffraction pattern. The EPA to DHA mixture was a 1:1 molar ratio. Values are mean±SEM (N=3). *p<0.05 versus control; ^{†}p<0.05 versus DHA; ^{§} p<0.05 versus Fenofib (Student-Newman-Keuls multiple comparisons test; overall ANOVA: p=0.0275, F=4.326).

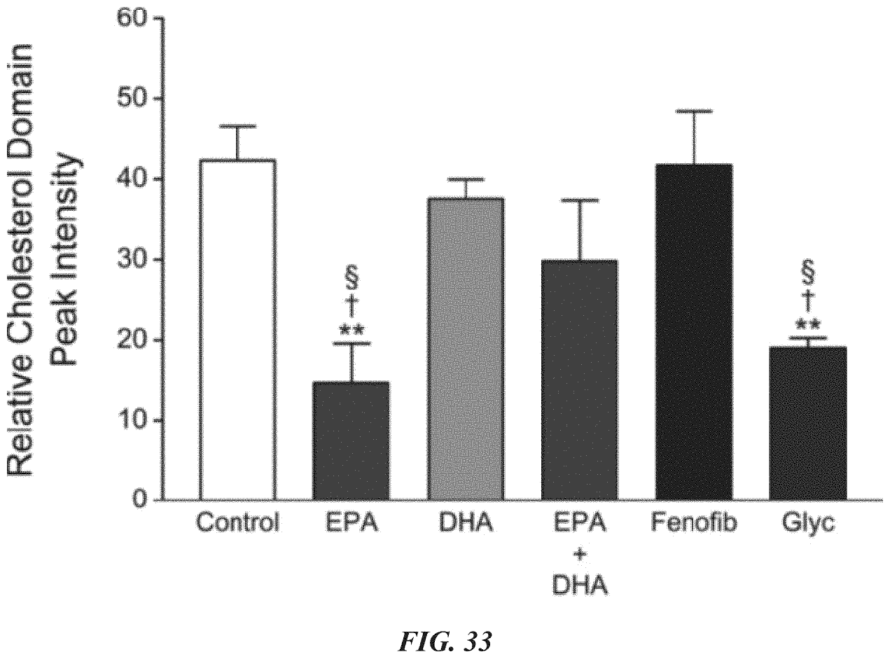

EPA treatment induced significant reduction in the domain peak intensity (65%, 14.6±5) compared to vehicle control. There were no significant changes in cholesterol domain size for membranes treated with either niacin (27.5±12.2) or gemfibrozil (46.9±12.6) as compared to vehicle treatment (Table 2); the cholesterol domain peaks associated with gemfibrozil treatment were numerically greater than vehicle by 11% than vehicle. A reduction in domain peak intensity was observed with niacin (−35%) but it was not statistically significant as compared to vehicle treated control samples (Table 2). Treatment with niacin was associated with substantial variation in cholesterol domain peak size compared to the other treatments. As a positive control, effects of EPA were compared to glycyrrhizin, a glycosylated sterol that has been shown to reduce cholesterol domains in model membranes. Like EPA, glycyrrhizin reduced the size of the integrated peaks associated with the cholesterol domains by 54.9% (19.1±1.2) in a highly reproducible and significant fashion (FIG. 33). Values are mean±SEM (N=3-6). **p<0.01 versus control; †p<0.05 versus DHA; § p<0.05 versus Fenofib (Student-Newman-Keuls multiple comparisons test; overall ANOVA: p=0.0010, F=7.624).

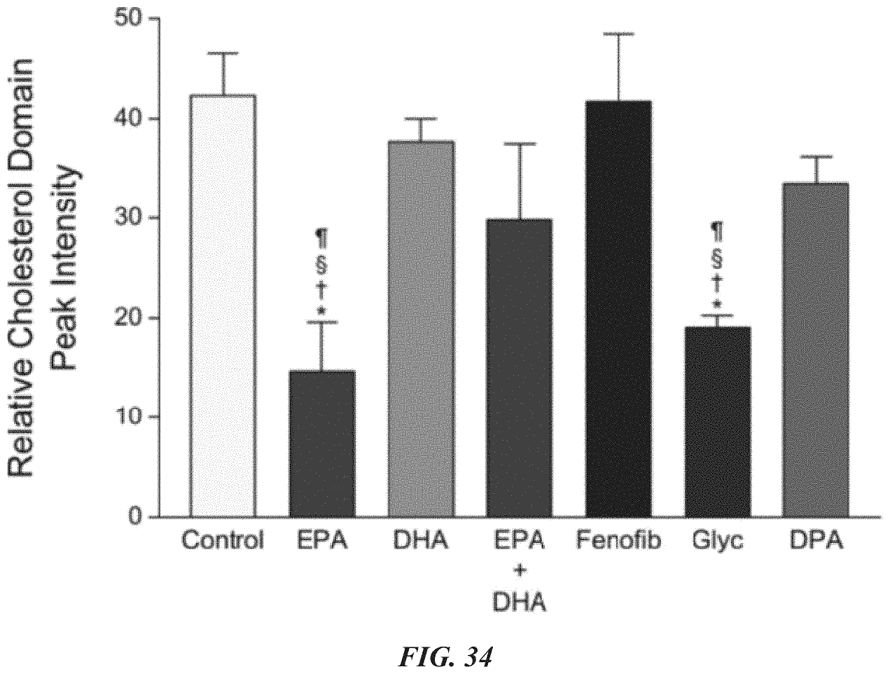

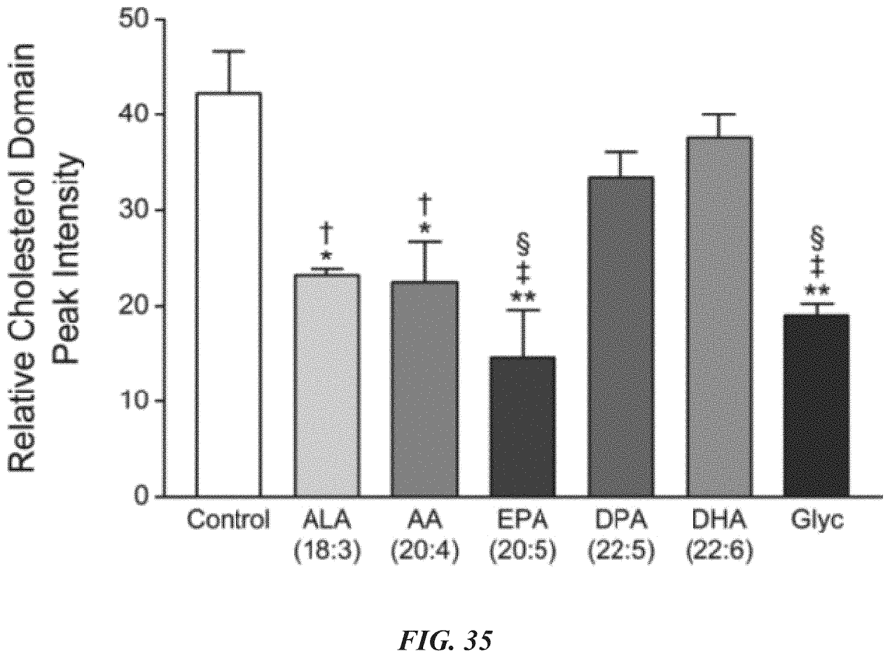

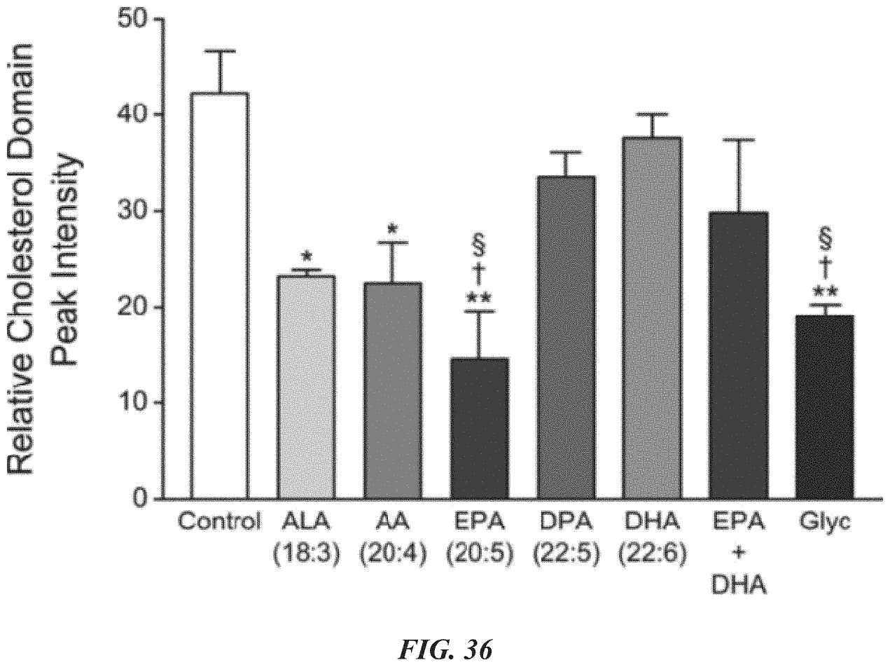

DPA did not reduce domains compared to EPA (Table 2, FIG. 34, 35, 36). Treatment with ALA (18:3; n−3) and AA (20:4; n−6) also significantly reduced domain peak intensity, although to lesser extents of −46.8% and −45.2%, respectively (p<0.05 versus control for both) (Table 2, FIGS. 35, 36). Values are mean±SEM (N=3-6). *p<0.01 versus control; †p<0.05 versus DHA; § p<0.01 versus Fenofib; ¶p<0.05 versus DPA (Student-Newman-Keuls multiple comparisons test; overall ANOVA: p=0.0946, F=1.864).

Dose-dependent effects of EPA on membrane cholesterol domains in model membrane prepared as binary mixtures of POPC and cholesterol at a 1.5:1 C/P mole ratio are reviewed in Table 3.

TABLE 3
Dose-Dependent Effects of EPA on
Membrane Cholesterol Domains.

| | Relative Cholesterol | % Change | † |
|---|---|---|---|
| Treatment* | Domain Peak Intensity | (vs. Control) | P Values |
| Vehicle | 42.3 ± 4.3 | — | — |
| EPA, 1:90 | 36.0 ± 3.1 | −15.0 ± 12.6 | 0.3010 |
| EPA, 1:60 | 29.5 ± 10.9 | −30.2 ± 27.9 | 0.3373 |
| EPA, 1:30 | 14.6 ± 5.0 | −65.5 ± 17.1 | 0.0139 |

Values are reported as mean ± SEM (N = 3-6).
*P values were calculated against vehicle-treated controls using an unpaired, two-tailed Student's t-test.
Values within parentheses after fatty acids denote carbon length and number of unsaturated bonds; omega-3 or omega-6 fatty acid classification.

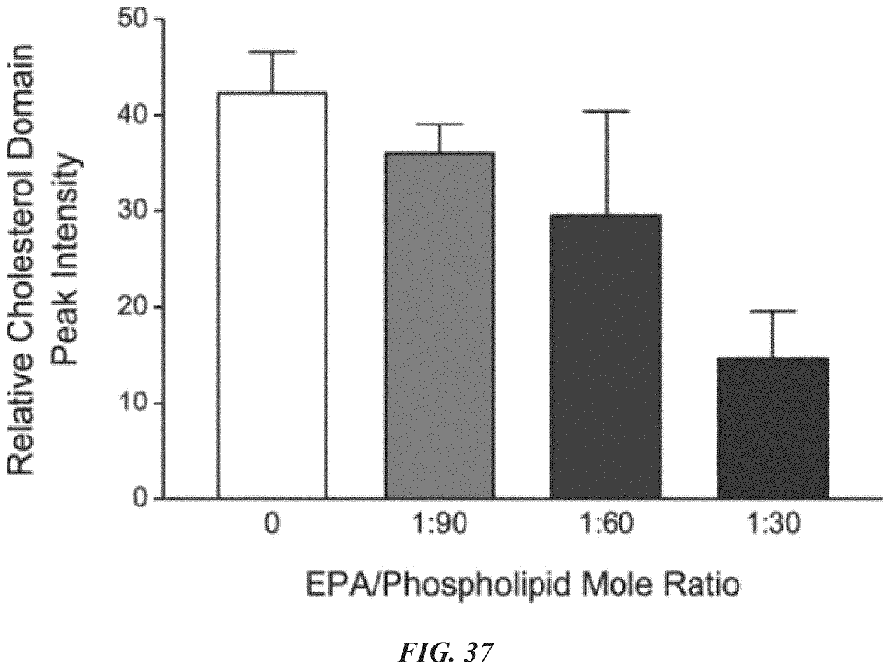

The reduction in cholesterol domain peak intensity with EPA was dose-dependent over a range of EPA to phospholipid mole ratios of 1:30 to 1:90 (Table 3, FIG. 37). Relative cholesterol peak intensity values were derived by integrating the second-order cholesterol domain peak and normalizing to total phospholipid peak area associated with a given diffraction pattern. Values are mean±SEM (N=3). ANOVA: p=0.0798, F=3.276.

### Example 10

The objective of this study was to compare the effects of EPA, fenofibrate, nicotinic acid, gemfibrozil, and vitamin E on the oxidation of small dense LDL (sdLDL) isolated from human subjects under conditions of hyperglycemia.

### Materials

Eicosapentaenoic acid (EPA), fenofibrate, gemfibrozil and nicotinic acid (niacin) were purchased from Sigma-Aldrich (Saint Louis, Mo.) and solubilized separately in ethanol to 1.0 mM under nitrogen atmosphere. Vitamin E (α-tocopherol) purchased from Sigma-Aldrich was prepared in ethanol at 1.0 mM (c=3.06×10⁴ M⁻¹ cm⁻¹ at 294 nm) prior to experimental use. Test compounds were further diluted in ethanol or aqueous buffer as needed. Phosphate Buffered Saline (PBS) purchased from Sigma-Aldrich was prepared in double distilled water at 10 mM (138 mM NaCl, 2.7 mM KCl, pH 7.4). Glucose prepared in PBS was added to test samples to achieve a final concentration of 11.0 mM (200 mg/dL).

### Methods

LDL and sdLDL were isolated from the plasma of healthy volunteers by iodixanol density gradient ultracentrifugation and adjusted to a final apolipoprotein concentration of 10 mg/mL. Sample aliquots (100 μg apolipoprotein each) were incubated with vehicle (ethanol), EPA, fenofibrate, gemfibrozil, or niacin (each at 10.0 μM) and adjusted to a final volume of 1.0 mL in glucose-treated PBS and incubated for 30 minutes at 37° C. in a shaking water bath. Lipid oxidation was initiated with 1.0 μM CuSO₄ and 100 μL of sample aliquots were combined with 1.0 mL thiobarbituric acid (0.5%), 10 μL trichloroacetic acid (10%), 10 μL BHT (35 mM in methanol), and 10 μL EDTA (5 mM) at different time points (up to 4 hr). Sample aliquots were incubated at 100° C. for 30 minutes and then assayed for the formation of thiobarbituric acid-reactive substances (TBARS), having a molar absorptivity (E) value of 1.56×10⁵ M⁻¹ cm⁻¹ at 532 nm and were derived principally from the reaction of thiobarbituric acid with malondialdehyde (MDA). Lipid oxidation was measured by the colorimetric detection of malondialdehyde (MDA), a marker of oxidative stress.

### Statistical Analyses

Data were presented as mean±SD for (n) separate samples or experiments. Differences between groups were analyzed using the unpaired, two-tailed Student's t-test (for comparisons between only two groups) or ANOVA followed by Student-Newman-Keuls multiple comparisons post hoc analysis (for comparisons between three or more groups). Only differences with probability values less than 0.05 were considered significant.

### Results

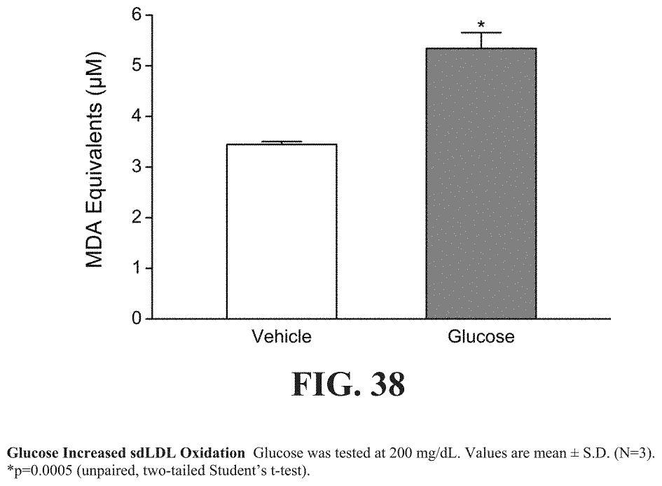

Exposure of sLDL to hyperglycemic conditions (200 mg/dL glucose) increased the sdLDL oxidation (formation of MDA) by 55% (from 3.4±0.1 to 5.3±0.3 μM) compared to non-glucose-treated controls (FIG. 38).

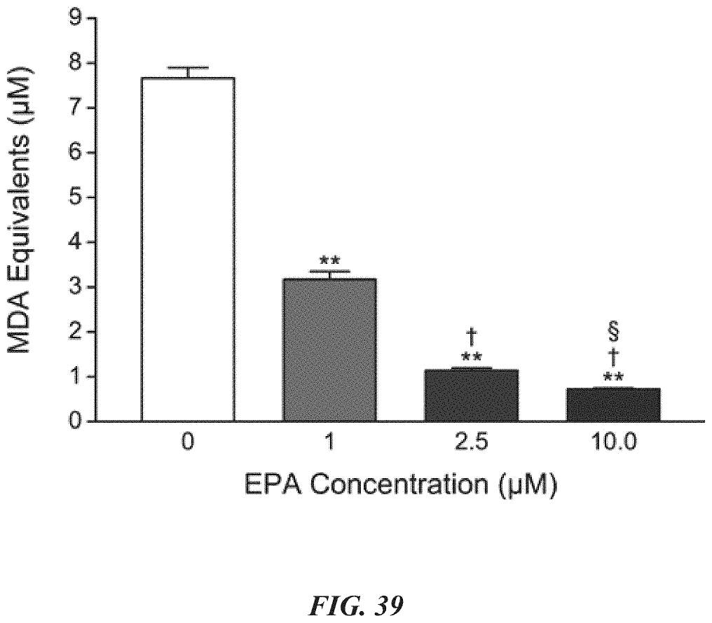

EPA treatment inhibited glucose-induced sdLDL oxidation in a dose-dependent fashion with an IC₅₀ less than 1.0 μM (FIG. 39). Further, at the highest concentration tested (10.0 μM), EPA inhibited sdLDL oxidation by 94% (5.3±0.3 μM to 0.2±0.1 μM) as compared to vehicle controls (FIG. 39). Values are mean±S.D. (N=3).**p<0.001 versus vehicle- (glucose-) treated control; †p<0.001 versus 1.0 μM EPA; § p<0.01 versus 2.5 μM EPA (Student-Newman-Keuls multiple comparisons test; overall ANOVA: p<0.0001, F=1358.7).

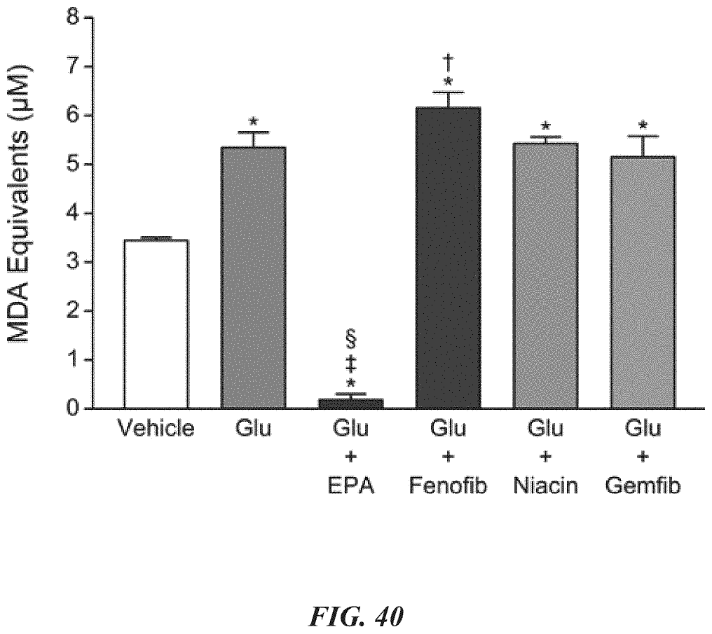

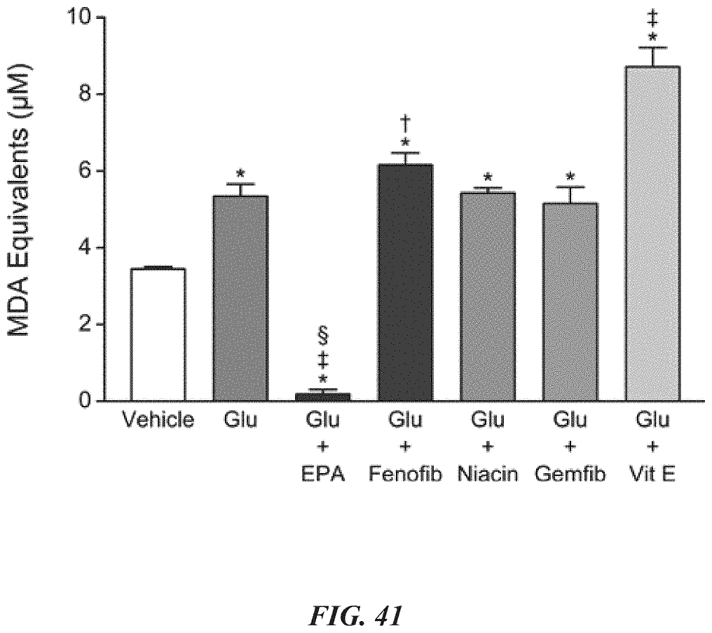

Treatment with fenofirbrate, niacin, and gemfibrozil did not inhibit glucose-induced sdLDL oxidation (FIG. 40 and FIG. 41). Values are mean±S.D. (N=3). *p<0.001 versus vehicle-treated control; †p<0.01 and ‡p<0.001 versus glucose (Glu; 200 mg/dL) alone; § p<0.001 versus Fenofib, Niacin, or Gemfib (with glucose) treatments (Student-Newman-Keuls multiple comparisons test; overall ANOVA: p<0.0001, F=211.23).

Vitamin E failed to inhibit sdLDL oxidation. Instead, vitamin E increased sdLDL oxidation by 63% as compared to glucose-treated controls (p<0.001) (FIG. 41). Values are mean±S.D. (N=3). *p<0.001 versus vehicle-treated control; ^{†}p<0.05 and ^{‡}p<0.001 versus glucose (Glu; 200 mg/dL) alone; ^{§} p<0.001 versus Fenofib, Niacin, or Gemfib (with glucose) treatments (Student-Newman-Keuls multiple comparisons test; overall ANOVA: p<0.0001, F=216.14).

### Example 11

The objectives of this study were to:
1) compare the time-dependent effects of pretreatment with EPA, alone or in combination with a statin (e.g., atorvastatin active metabolite, also referred to herein as “ATM”), to pretreatment with other triglyceride-lowering agents such as fenofibrate, niacin and gemfibrozil and DHA on endothelial cell function as indicated by changes in nitric oxide (NO) and nitroxidative (ONOO⁻) stress release under conditions of oxidative stress using oxidized LDL (oxLDL);
2) test the effects of oxLDL treated with EPA, alone or in combination with ATM, on endothelial cell function compared to those of treatment with DHA; and
3) test the effects of pretreating LDL with EPA, alone or in combination with ATM, and then subjecting the pretreated LDL to oxidation, compared to those of DHA.

The omega-3 fatty acid, cis-5,8,11,14,17-eicosapentaenoic acid (EPA) and docosahexaenoic acid (DHA) were purchased from Sigma-Aldrich (Saint Louis, Mo.) and solubilized in redistilled ethanol to 11 mM under nitrogen atmosphere. EPA and DHA stock solutions were stored at −20° C. until use. Fenofibrate, gemfibrozil and nicotinic acid were purchased from Toronto Research Chemicals (North York, Ontario, Canada) and solubilized in ethanol at 1 mM; subsequent dilutions were prepared in ethanol or aqueous buffer as needed. Atorvastatin ortho- (o-) hydroxy (active) metabolite (ATM) was also purchased from Toronto Research Chemicals (North York, Ontario, Canada) and solubilized in ethanol at 1.0 mM.

Human umbilical vein endothelial cells (HUVECs) were obtained from healthy, female donors and isolated into primary, proliferating cell cultures (Clonetics, San Diego, Calif.). The cells were incubated in 95% air/5% CO₂ at 37° C. and passaged by an enzymatic (trypsin) procedure. The confluent cells (4 to 5×10⁵ cells/35 mm dish) were plated with minimum essential medium containing 3 mM L-arginine and 0.1 mM BH₄ [(6R)-5,6,7,8-tetrahydrobiopterin]. Before experimental use, the cells (from second or third passage) were rinsed twice with Tyrode-HEPES buffer with 1.8 mM CaCl₂.

Venous blood was collected from healthy volunteers into vacutainer tubes containing sodium EDTA. Plasma was separated by centrifugation at 3000 g for 25 min at 4° C. and adjusted to a density of 1.020 g/mL with KBr. Triglyceride-rich lipoproteins (TGRL) and LDL fractions were isolated by sequential centrifugation at 70,000 rpm at 4° C. in a Beckman LE-80 ultracentrifuge using a Beckman 50.4Ti rotor (Beckman Coulter, Inc., Fullerton, Calif.). The TGRL fraction (with a relative density of <1.020) was aspirated from the top of the centrifugate and discarded, leaving an LDL-enriched infranate (with a relative density of 1.020-1.063). The LDL subfraction was retained for additional experimentation. Plasma and lipoprotein fractions were maintained at 4° C. and protected from excessive exposure to light. Prior to oxidation experiments, EDTA was removed from the lipoprotein fraction using PD-10 desalting columns (GE Healthcare, Piscataway N.J.). After equilibration of the column with phosphate buffered saline (PBS), 2.5 mL of the lipoprotein fraction was loaded on the column and the flow-through discarded. The lipoprotein was then eluted with 3 mL of PBS. The LDL fractions were further diluted with PBS to obtain a 100 μg/mL apoB100 concentration.

LDL underwent oxidation following the addition of 10 μM CuSO₄. To measure oxidation, 100 μL aliquots were removed and combined with 1.0 mL thiobarbituric acid (0.5%), 10 μL trichloroacetic acid (5%), 10 μL BHT (5 mg/mL in methanol), and 10 μL EDTA (5 mM). Sample aliquots were incubated at 100° C. for 20 min and then assayed for the formation of TBARS, which have a molar absorptivity (ε) value of 1.56×10⁵ M⁻¹ cm⁻¹ at 532 nm and are derived principally from the reaction of thiobarbituric acid with MDA, a reactive aldehyde produced by LDL oxidation. Sample TBARS concentrations were determined spectrophotometrically by measuring sample absorbances against a standard curve derived from the hydrolysis of 1,1,3,3-tetramethoxypropane.

Concurrent measurements of NO and ONOO⁻ were performed with tandem electrochemical nanosensors combined into one working unit with a total diameter of 200-400 nm. Their design is based on previously developed and chemically modified carbon-fiber technology. Each of the nanosensors was made by depositing a sensing material on the tip of a carbon fiber (length 4 to 5 μm, diameter 100-200 nm). The fibers were sealed with nonconductive epoxy and electrically connected to copper wires with conductive silver epoxy. Conductive films of polymeric Ni(II) tetrakis (3-methoxy-4-hydroxyphenyl) porphyrin and Mn(III) [2.2] paracyclophanyl-porphyrin were used for the NO and ONOO⁻ sensors, respectively.

The amperometric method (with a response time of 0.1 ms) provides a quantitative signal (current) that is directly proportional to changes (from basal levels) in NO or ONOO⁻ concentration. Amperometric measurements were performed with a Gamry III double-channel potentiostat. Basal NO or ONOO⁻ levels were measured by differential pulse voltammetry in separate experiments.

All measurements of NO and ONOO⁻ were performed on intact endothelial cells. The NO/ONOO⁻ nanosensor module was positioned 5±2 m from the surface of each individual endothelial cell using a computer-controlled M3301 micromanipulator (x-y-z resolution of 0.2 m) and microscope (both from World Precision Instruments, Berlin, Germany) fitted with a CD camera. After establishing a background current, EPA and other TG-lowering agents (fenofibrate, niacin, and gemfibrozil), in the absence and presence of different statins, were added to the cells. Cells will then be treated with calcium ionophore (to maximally stimulate the release of nitrous molecules) and monitored continuously for changes in amperometric current.

Data are presented as mean±S.D. for (N) separate samples or treatment groups. Differences between groups were analyzed using the two-tailed, Student's t-test (for comparisons between only two groups) or ANOVA followed by Dunnett or Student-Newman-Keuls multiple comparisons post-hoc analysis (for comparisons between three or more groups). Alpha error was set to 0.05 in this study.

To assess the time-dependent effects of pretreatment (Objective 1), HUVECs were treated with vehicle or oxLDL for up to three hours following EPA pretreatment for 30 min-alone or in combination with ATM-versus DHA, fenofibrate, niacin and gemfibrozil. NO and ONOO⁻ release were measured following stimulation with calcium. Cells were incubated with the various test agents at 10 μM and then challenged with oxLDL at 11 mg/dL (based on protein concentration) prior to stimulating maximal NO and ONOO⁻ release with 1.0 μM calcium ionophore. Control groups were treated with vehicle alone in the absence or presence of oxLDL. The NO/ONOO⁻ ratio was calculated as the arithmetic quotient of separate NO and ONOO⁻ measurements. HUVECs were pretreated for 30 min with the different compounds followed by exposure to oxLDL for up to 3 hr before stimulation of NO and ONOO⁻ release with calcium.

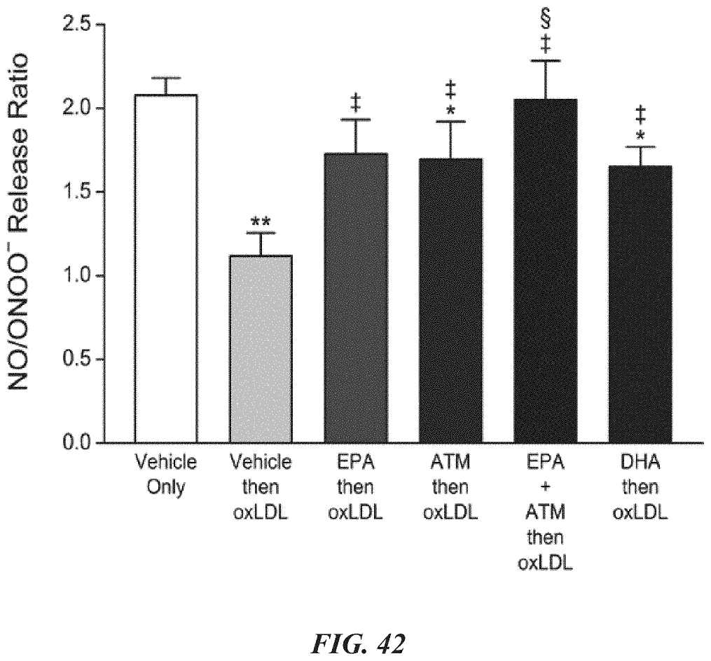

As shown in Table 4 and FIG. 42, all of the tested treatments improved endothelial function for up to 3 hrs if applied prior to the addition of oxLDL. The greatest improvement in the NO/ONOO⁻ ratio was observed with the combination of EPA and ATM as it increased this ratio by 86% from 1.12±0.14 with vehicle to 2.05±0.23 with the combination treatment (p<0.001). This increase with the combination was significant as compared to DHA (1.68±0.12) or ATM (1.69±0.23) alone (p<0.05) and even EPA alone (1.73±0.20). It was also significant compared to fenofibrate (1.51±0.19), gemfibrozil (1.65±0.13) and niacin (1.45±0.14). All of the treatments improved the NO/ONOO⁻ ratio as compared to vehicle treatment alone. Cells were incubated with the various test agents at 10 LM and then challenged with oxLDL at 11 mg/dL (based on protein concentration) prior to stimulating maximal NO and ONOO− release with 1.0 μM calcium ionophore. Control groups were treated with vehicle alone in the absence or presence of oxLDL. The NO/ONOO− ratio was calculated as the arithmetic quotient of separate NO and ONOO− measurements. HUVECs were pretreated for 30 min with the different compounds followed by exposure to oxLDL for up to 3 hr before stimulation of NO and ONOO− release with calcium. Values are mean±S.D. (N=3-4). *p<0.05 and **p<0.001 versus vehicle only (no oxLDL); ‡<0.001 versus vehicle-then-oxLDL; § p<0.05 versus separate ATM or DHA treatments (Student-Newman-Keuls multiple comparisons test; overall ANOVA: p<0.0001, F=13.631).

TABLE 4
Effects of oxLDL on NO and ONOO⁻ release from
HUVECs pretreated with EPA, ATM, EPA/ATM combination,
DHA, fenofibrate, gemfibrozil, or nicotinic acid.

| Treatment | NO | ONOO⁻ | NO/ONOO⁻ Ratio |
|---|---|---|---|
| Vehicle | 431 ± 20 | 208 ± 3 | 2.08 ± 0.10 |
| Vehicle, oxLDL | 294 ± 25** | 263 ± 23* | 1.12 ± 0.14** |
| EPA, oxLDL | 394 ± 43^{‡} | 228 ± 10 | 1.73 ± 0.20*^{‡} |
| ATM, oxLDL | 382 ± 25^{‡} | 226 ± 26 | 1.69 ± 0.23*^{†} |
| EPA/ATM, oxLDL | 428 ± 25^{‡§} | 209 ± 21^{†} | 2.05 ± 0.23^{‡¶} |
| DHA, oxLDL | 378 ± 5^{†} | 229 ± 16 | 1.68 ± 0.12*^{†} |
| Fenofib, oxLDL | 349 ± 28*^{†} | 231 ± 22 | 1.51 ± 0.19*^{†} |
| Gemfib, oxLDL | 377 ± 24^{†} | 228 ± 11 | 1.65 ± 0.13*^{†} |
| Niacin, oxLDL | 346 ± 17*^{†} | 238 ± 20 | 1.45 ± 0.14*^{†} |

Values are reported as mean ± S.D. (N = 3-4).
*p < 0.05 and
**p < 0.001 versus vehicle-only treatment;
^{†}p < 0.05 and
^{‡}p < 0.001 versus vehicle, oxLDL treatment;
^{§}p < 0.05 versus cognate Fenofib or Niacin treatments;
^{¶}p < 0.05 versus all other treatments (Student-Newman-Keuls multiple comparisons post hoc test; overall ANOVA-NO release data: p < 0.0001, F = 9.431; ONOO⁻ release data: p = 0.0099, F = 3.132; NO/ONOO⁻ ratio data: p < 0.0001, F = 9.972).

To test the effects of EPA—alone or in combination with ATM—versus DHA on EC function (Objective 2), HUVECs were treated with vehicle or oxLDL that was treated for 30 min with EPA, alone or in combination with ATM, or DHA. After 60 min exposure to the oxLDL that had been treated with EPA, EPA/ATM or DHA, the cells were stimulated with calcium to measure release of NO and ONOO⁻.

Cells were co-incubated with the various test agents, each at 10 μM, and oxLDL at 11 mg/dL (based on protein concentration) prior to stimulating maximal NO and ONOO⁻ release with 1.0 μM calcium ionophore. Control groups were treated with vehicle alone in the absence or presence of oxLDL. The NO/ONOO⁻ ratio was calculated as the arithmetic quotient of separate NO and ONOO⁻ measurements. HUVECs were treated with oxLDL that had been already treated with the different compounds for 30 min. After 60 min, NO and ONOO⁻ release was induced with 1.0 μM calcium ionophore stimulation, and the amounts of released NO and ONOO⁻ were measured.

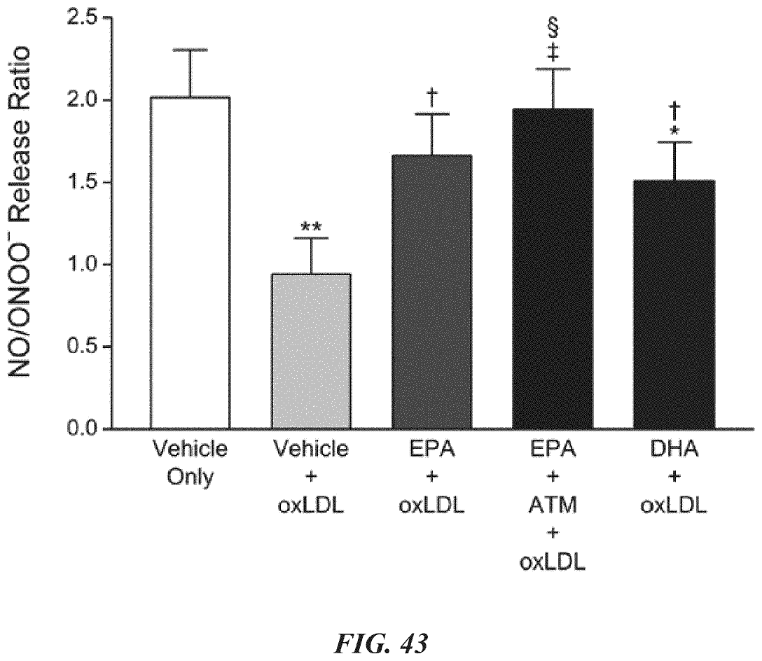

As shown in Table 5 and FIG. 43, the greatest improvement in endothelial function were observed with the combination of EPA and ATM (1.94±0.25) by two-fold as compared to vehicle+oxLDL (0.94±0.22). This effect was significant as compared to DHA+oxLDL (1.51±0.30) or EPA+oxLDL alone (1.66±0.25). Values are mean±S.D. (N=3-4). *p<0.05 and **p<0.001 versus vehicle only (no oxLDL); †p<0.05 and ‡p<0.001 versus vehicle+oxLDL; ^{§} p<0.05 versus DHA+oxLDL (Student-Newman-Keuls multiple comparisons test; overall ANOVA: p<0.0001, F=13.958).

TABLE 5
Comparative effects of EPA, DHA, and EPA/ATM
combination treatment on NO and ONOO⁻ release
from HUVECs treated concomitantly with oxLDL.

| Treatment | NO | ONOO⁻ | NO/ONOO⁻ Ratio |
|---|---|---|---|
| Vehicle | 426 ± 42 | 211 ± 22 | 2.02 ± 0.29 |
| Vehicle + oxLDL | 262 ± 45** | 278 ± 43* | 0.94 ± 0.22** |
| EPA + oxLDL | 385 ± 43^{‡} | 232 ± 24^{†} | 1.66 ± 0.25^{†} |
| EPA/ATM + oxLDL | 416 ± 36^{‡} | 215 ± 20^{†} | 1.94 ± 0.25^{‡§} |
| DHA + oxLDL | 362 ± 39^{‡} | 240 ± 27 | 1.51 ± 0.23*^{†} |

Values are reported as mean ± S.D. (N = 4-7).
*p < 0.05 and
**p < 0.001 versus vehicle-only treatment;
^{†}p < 0.05 and
^{‡}p < 0.001 versus vehicle + oxLDL treatment;
^{§}p < 0.05 versus cognate DHA treatment (Student-Newman-Keuls multiple comparisons post hoc test; overall ANOVA-NO release data: p < 0.0001, F = 15.700; ONOO⁻ release data: p = 0.0166, F = 3.969; NO/ONOO⁻ ratio data: p < 0.0001, F = 13.958).

To test the effects of EPA pretreatment of HUVECs combined with the effects of EPA pretreatment of LDL undergoing auto-oxidation (Objective 3), HUVECs were pretreated with EPA—alone or in combination with ATM—or DHA for 30 min. The cells were then exposed to native LDL that had been pretreated with these same agents under conditions of auto-oxidation for 2 h. After 60 min, NO and ONOO⁻ release were measured after being induced with addition of calcium.

Cells were co-incubated with the various test agents, each at 10 μM, and oxLDL or native LDL, each at 11 mg/dL (based on protein concentration), prior to stimulating maximal NO and ONOO⁻ release with 1.0 μM calcium ionophore. Control groups were treated with vehicle alone in the absence or presence of oxLDL. The NO/ONOO⁻ ratio was calculated as the arithmetic quotient of separate NO and ONOO⁻ measurements. HUVECs were pretreated with the different agents for 30 min. The cells were then exposed to LDL that had been pretreated with the different compounds under conditions of auto-oxidation for 30 min. After 60 min, NO and ONOO⁻ release were measured after being stimulated with addition of calcium.

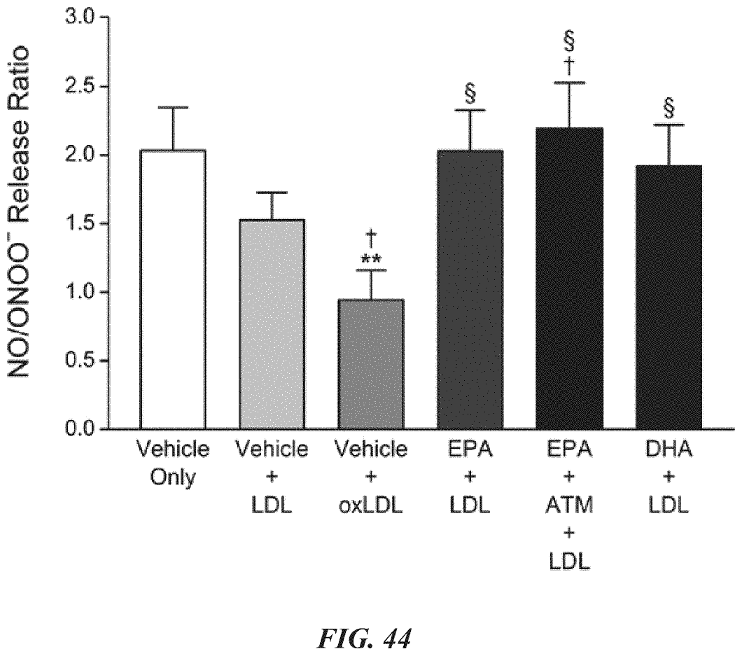

As shown in Table 6 and FIG. 44, the greatest improvement in endothelial function was observed with the combination of EPA and ATM+LDL (auto-oxidized) (2.19±0.33) as compared to vehicle+oxLDL (0.95±0.22); the improvement was not significant compared to vehicle+LDL (auto-oxidized) (1.53±0.20). This effect was numerically greater as compared to DHA (1.92±0.30) and EPA (2.03±0.30) alone but not in a statistically significant fashion. Values are mean±S.D. (N=3-4). **p<0.001 versus vehicle only (no oxLDL); †p<0.01 versus vehicle+LDL; ^{§} p<0.001 versus vehicle+oxLDL (Student-Newman-Keuls multiple comparisons test; overall ANOVA: p<0.0001, F=13.436).

TABLE 6
Comparative effects of EPA, DHA, and EPA/ATM combination
pretreatment on NO and ONOO⁻ release from HUVECs
treated concomitantly with native LDL undergoing oxidation.

| Treatment | NO | ONOO⁻ | NO/ONOO⁻ Ratio |
|---|---|---|---|
| Vehicle | 430 ± 43 | 212 ± 25 | 2.03 ± 0.32 |
| Vehicle + LDL | 362 ± 30 | 237 ± 24 | 1.53 ± 0.20 |
| Vehicle + oxLDL | 263 ± 45**^{‡} | 278 ± 43*^{†} | 0.95 ± 0.22**^{†} |
| EPA + LDL | 419 ± 37^{§} | 207 ± 24^{§} | 2.03 ± 0.30^{§} |
| EPA/ATM + LDL | 444 ± 54^{†§} | 202 ± 18^{§} | 2.19 ± 0.33^{†§} |
| DHA + LDL | 406 ± 40^{§} | 212 ± 26^{§} | 1.92 ± 0.30^{§} |

Values are reported as mean ± S.D. (N = 4-7).
*p < 0.01 and
**p < 0.001 versus vehicle-only treatment;
^{†}p < 0.05 and
^{‡}p < 0.001 versus vehicle + LDL treatment;
^{§}p < 0.01 versus vehicle + oxLDL treatment (Student-Newman-Keuls multiple comparisons post hoc test; overall ANOVA-NO release data: p < 0.0001, F = 15.995; ONOO⁻ release data: p = 0.0017, F = 5.412; NO/ONOO⁻ ratio data: p < 0.0001, F = 13.436).

In all of the different test conditions, the best improvement in endothelial function was observed with the combination of EPA and ATM. This was evident when the treatments were added prior to oxLDL exposure (i.e. HUVEC pretreatment) or to oxLDL that is then added to the HUVECs. Finally, the combination of EPA and ATM was also best when added to the LDL while undergoing oxidative conditions.

### Example 12

An experiment to study the antioxidant effect of EPA in small dense LDL (“sdLDL”) was performed. sdLDL was isolated from human plasma by isopycnic centrifugation, separated into test samples of 200 μg/mL, and incubated at 37° C. for 30 min in the absence (vehicle) or presence of EPA, fenofibrate, niacin, or gemfibrozil, each at 10.0 μM. All samples, with the exception of non-oxidized sdLDL controls, were subjected to copper-induced oxidation for 1 hour. Human umbilical vein endothelial cells (HUVECs) were incubated with the various sdLDL samples, stimulated with calcium, and monitored for nitric oxide (NO) and peroxynitrite (ONOO⁻) release using nanosensor technology.

EPA treatment reduced sdLDL oxidation by >90% (p<0.001) compared to vehicle treatment alone. When applied directly to HUVECs, vehicle-treated, oxidized sdLDL reduced NO release by 20% as compared to non-oxidized sdLDL (from 758±40 to 610±43 nM). Following exposure to EPA-treated, oxidized sdLDL, however, HUVEC NO release (931±59 nM) increased by 53% and 23% as compared to oxidized LDL and non-oxidized sdLDL treatments, respectively. None of the other TG-lowering agents inhibited sdLDL oxidation, resulting in reduced NO release. In HUVECs challenged with oxidized sdLDL, pretreated with fenofibrate, niacin, or gemfibrozil, NO release was reduced by 21%, 45%, and 33%, respectively, as compared to effects observed with vehicle-treated, oxidized sdLDL (p<0.05).

These data demonstrate that EPA pretreatment reduced sdLDL oxidation and improved endothelial function as compared to other TG-lowering agents.

### Example 13

An experiment to study the differential effects of EPA and DHA in small dense LDL (“sdLDL”) oxidation rates was performed. More specifically, the time-dependent effects of EPA, DHA, and their combination on sdLDL oxidation were compared.

sdLDL was isolated from the plasma of healthy volunteers by iodixanol density gradient centrifugation and adjusted to a final apolipoprotein concentration of 2 mg/mL. Sample aliquots (100 μg apolipoprotein) were incubated with EPA, DHA, or EPA+DHA (each at 10 μM) for 30 minutes at 37° C. and then treated with 10 μM CuSO₄ to initiate lipid oxidation. sdLDL oxidation was measured spectrophotometrically as a function of malondialdehyde (MDA) formation at various intervals over 4 hours. Vitamin E was also tested as a positive control. Experiments were conducted in triplicate and reported as mean±SD.

Oxidation of sdLDL occurred in a logarithmic fashion with vehicle treatment, as evidenced by a greater than 10-fold increase in MDA levels at 1 hour. EPA inhibited sdLDL oxidation throughout the incubation period, with a 92% reduction (9.6±0.1 to 0.8±0.03 μM) observed at 4 hours. The antioxidant activity of DHA treatment diminished over time, with only a 51% reduction (to 4.7±0.1 μM; p<0.001) observed at 4 hours under identical conditions. EPA+DHA produced an intermediate effect on the sdLDL oxidation rate, reducing MDA levels by 79% (to 2.0±0.4 μM; p<0.001). By contrast, no significant antioxidant effect was observed with vitamin E over the entire experimental time course.

These data demonstrate that EPA and DHA have different in vitro antioxidant effects on sdLDL oxidation at pharmacologic concentrations. Over time, the antioxidant effects of EPA were significantly greater than DHA, alone or in combination with EPA, and were not reproduced with vitamin E.

From the foregoing, it will be appreciated that specific embodiments of the invention have been described herein for purposes of illustration, but that various modifications may be made without deviating from the scope of the invention. Accordingly, the invention is not limited except as by the appended claims.

1. A method of reducing or preventing sdLDL oxidation in a subject in need thereof, the method comprising administering to the subject a pharmaceutical composition comprising eicosapentaenoic acid or a derivative thereof.

2. The method of claim 1, wherein the pharmaceutical composition comprises at least 80%, at least 90%, at least 95%, or at least 96%, by weight of all fatty acids (and/or derivatives thereof) present, eicosapentaenoic acid or a derivative thereof.

3. The method of claim 1, wherein the pharmaceutical composition comprises no docosahexaenoic acid or esters thereof.

4. The method of claim 1, wherein the reduction or prevention occurs by a free radical chain-breaking mechanism.

5. The method of claim 1 further comprising determining a baseline oxidized sdLDL level in the subject prior to administering to the subject the pharmaceutical composition.

6. The method of claim 5 further comprising determining a second oxidized sdLDL level in the subject after administering to the subject a pharmaceutical composition comprising eicosapentaenoic acid or a derivative thereof, wherein the second oxidized sdLDL level is not greater than, not significantly greater than, or lower than the baseline oxidized sdLDL level and/or wherein the second oxidized sdLDL level is not greater than, not significantly greater than, or lower than the baseline oxidized sdLDL level in comparison to a second subject who has not received the pharmaceutical composition.

7. The method of claim 6, wherein the method further comprises administering o-hydroxyatorvastatin to the subject.

8. The method of claim 7, wherein the second oxidized sdLDL level is not greater than, not significantly greater than, or lower than the baseline oxidized sdLDL level in comparison to a second subject who has received the o-hydroxyatorvastatin but not the pharmaceutical composition.

9. The method of claim 7, wherein the second oxidized sdLDL level is not greater than, not significantly greater than, or lower than the baseline oxidized sdLDL level in comparison to a second subject who has received the pharmaceutical composition but not the o-hydroxyatorvastatin.

10. The method of claim 1, wherein the subject has a baseline triglyceride level of at least 500 mg/dL.

11. The method of claim 1, wherein the subject has a baseline triglyceride level of about 200 mg/dL to 499 mg/dL.

12. The method of claim 11, wherein the subject is on statin therapy, optionally stable statin therapy.

13. The method of claim 1, wherein the subject is hyperglycemic or is diabetic.

14.-20. (canceled)
